(19) United States
(12) Patent Application Publication
(10) Pub. No.: US 2024/0277890 A1
(43) Pub. Date: Aug. 22, 2024

(54) ANTIMICROBIAL ARTICLES COMPRISING POLYURETHANE

(71) Applicant: Amicoat AS, Tromsø (NO)

(72) Inventors: Andreas Spiegelberg, Horgen (CH); Wenche Stensen, Kvaløysletta (NO); John Sigurd Svendsen, Kvaløysletta (NO)

(21) Appl. No.: 18/568,478

(22) PCT Filed: Jun. 14, 2022
(86) PCT No.: PCT/EP2022/066234

§ 371 Date: Dec. 8, 2023

(30) Foreign Application Priority Data
Jun. 14, 2021 (GB) 2108465.2

(51) Int. Cl. A61L 26/00

(52) U.S. Cl. CPC A61L 26/0019; A61L 26/0028

(57) ABSTRACT

The present invention provides an antimicrobial article comprising polyurethane, wherein said polyurethane is impregnated with a compound of Formula (I)

AA-AA-AA-X—Y (I)

The invention also provides polyurethane impregnated with a compound of Formula (I). The invention also provides compositions comprising at least one organic solvent, a thermoplastic polyurethane and a compound of Formula (I), and coatings produced using such compositions. The invention also provides methods of producing polyurethane impregnated with a compound of Formula (I), medical devices incorporating polyurethane impregnated with a compound of Formula (I), and medical uses of polyurethane impregnated with a compound of Formula (I).

This invention relates generally to the field of antimicrobial articles, such as wound dressings and other articles that may be susceptible to microbial contamination. More particularly, the invention relates to antimicrobial articles comprising certain antimicrobial compounds. Such articles have medical uses, such as in the fields of wound care and infection control. The invention also relates to antimicrobial coatings and coating compositions comprising certain antimicrobial compounds.

Skin plays a plays a vital role as a protective barrier from the external environment, for example protecting against infection by bacteria. It is important for the integrity of skin to be maintained, and if the integrity of skin is compromised, for example by the presence of a wound, it is important that the skin heals effectively to reinstate its integrity.

In order to protect wounds during the wound healing process, and to promote wound healing, wound dressings or the like are typically applied to wounds. During the wound healing process, it can be important for wound dressings to be capable of absorbing wound exudate and maintaining proper levels of moisture in the wound. Proper levels of moisture can be important for re-epithelialization and tissue remodeling during the wound healing process.

There are many different types of wound dressing, and in many cases (e.g. in the care of exuding wounds) it is important for wound dressings to capable of maintaining a moist wound environment conducive to wound healing. Polyurethane dressings represent one example of a type of wound dressing that is often used in this regard. Polyurethane-based dressings (e.g. polyurethane foam dressings) are typically capable of absorbing wound exudate and maintaining appropriate levels of moisture at the wound.

However, a major problem in the field of wound care is that, despite the application of wound dressings, wounds often get infected, for example by bacteria such as Staphylococci. Wound healing is typically impaired as a result of infection, with symptoms often including increased pain, swelling, and redness or, in more severe cases, nausea, chills, or fever. In addition to the wound (or wound bed) itself being infected, it is often the case that the wound dressing itself becomes colonized with microbes (typically bacteria). This is of course undesirable as it can result in a “bacteria-rich” environment in the wound and its surroundings, thereby impairing the healing process. Other articles such as other medical devices can also be susceptible to microbial contamination.

In an effort to counter bacterial wound infections, a variety of commercial wound dressing products have been launched in which the wound dressings comprise an antimicrobial agent. By way of an example, polyurethane foam dressings that are impregnated with the heavy metal silver (Ag) have been developed and are commercially available. Silver ions (Ag⁻) are capable of reacting with many biomolecules such as DNA, amino acids, and compounds with sulfhydrul groups, and can elicit cytotoxic effects. One example of a commercially available polyurethane foam wound dressing impregnated with silver is Optifoam® Gentle AG+ (Medline Industries Inc., USA).

However, although silver-impregnated dressings may be useful to combat some wound infections, there are certain disadvantages to employing heavy metals such as silver as an antimicrobial agent. For example, as silver is an element, it is not biodegradable, and it is well-known that the accumulation of silver (a heavy metal) is not desirable from an environmental perspective. The use of silver-impregnated dressings could lead to an undesirable environmental accumulation of silver (Ag), for example at sites at which such dressings are disposed of. In addition, there have been reports of bacterial infections that are resistant to treatment with silver, so silver-impregnated dressings would not be antimicrobially effective against such infections.

Thus, what is needed in the art are alternative, and preferably advantageous, wound dressings (and other articles that may be susceptible to microbial contamination) that have desirable features of other wound dressings (or other articles that may be susceptible to microbial contamination) and that additionally are provided with a compound that has good antimicrobial activity. Such dressings (or other articles) would be useful for controlling (or preventing) infections (e.g. bacterial infections), e.g. in wounds, and would preferably keep themselves (i.e. keep the dressings or articles themselves) infection-free (or “self-sterile”) by virtue of the presence of the antimicrobial compound.

The present inventors have advantageously provided such alternative wound dressings and other antimicrobial articles. For example, the inventors have developed polyurethane wound dressings that are provided with a certain class of short cationic antimicrobial peptide that has an excellent ability to leach out of the polyurethane dressing material and inhibit bacterial growth. Without wishing to be bound by theory, it believed that such dressings have a number of advantages over silver-impregnated polyurethane foam dressings, for example as (i) the cationic antimicrobial peptides are biodegradable and thus do not have the environmental concerns associated with silver impregnated dressings and (ii) bacterial resistance to cationic antimicrobial peptides is very rare (as it is unlikely that radical changes to lipid membranes that are the target for such peptides could occur to prevent the cytotoxic effect). The present inventors have also advantageously provided polyurethane coatings that are provided with such short cationic antimicrobial peptides. Such coatings are useful for the coating of articles (or surfaces) that may be exposed to (or susceptible to) bacterial contamination (e.g. medical devices such as catheters and the like).

Thus, in one aspect, the present invention provides an antimicrobial article comprising polyurethane, wherein said polyurethane is impregnated with a compound of Formula (I)

AA-AA-AA-X—Y (I)
wherein, in any order, 2 of said AA (amino acid) moieties are cationic amino acids, preferably lysine or arginine but may be histidine or any non-genetically coded or modified amino acid carrying a positive charge at pH 7.0, and 1 of said AA is an amino acid with a large lipophilic R group, the R group having 14-27 non-hydrogen atoms and preferably containing 2 or more, e.g. 2 or 3, cyclic groups which may be fused or connected, these cyclic groups will typically comprise 5 or 6 non-hydrogen atoms, preferably 6 non-hydrogen atoms (in the case of fused rings of course the non-hydrogen atoms may be shared);
X is a N atom, which may be but preferably is not substituted by a branched or unbranched C₁-C₁₀ alkyl or aryl group, e.g. methyl, ethyl or phenyl, and this group may incorporate up to 2 heteroatoms selected from N, O and S; and
Y is selected from the group consisting of R₁—R₂—R₃.
R₁—R₂—R₂—R₃, R₂—R₂—R₁—R₃, R₁—R₃, and R₄
wherein:
R₁ is C, O, S or N, preferably C;
R₂ is C;
each of R₁ and R₂ may be substituted by C₁-C₄ alkyl groups or unsubstituted, preferably Y is —R₁—R₂—R₃ (in which R₁ is preferably C) and preferably this group is not substituted, when Y is —R₁—R₂—R₂—R₃ or R₂—R₂—R₁—R₃ then preferably one or more of R₁ and R₂ is substituted;
R₃ is a group comprising 1 to 3 cyclic groups each of 5 or 6 non-hydrogen atoms (preferably all C atoms but optionally also containing N, O or S), 2 or more of the cyclic groups may be fused; one or more of the rings may be substituted and these substitutions may, but will typically not, include polar groups, suitable substituting groups include halogens, preferably bromine or fluorine and C₁-C₄ alkyl groups; R₃ incorporates a maximum of 15 non-hydrogen atoms, preferably 5-12, most preferably it is phenyl; and
R₄ is an aliphatic moiety having 2-20 non-hydrogen atoms, preferably these are carbon atoms but oxygen, nitrogen or sulphur atoms may be incorporated, preferably R₄ comprises 3-10, most preferably 3-6 non-hydrogen atoms and the moiety may be linear, branched or cyclic. If the R₄ group comprises a cyclic group this is preferably attached directly to the nitrogen atom of X.

Preferred compounds incorporate an R₄ group which is linear or branched, in particular a linear or branched alkyl group including ethyl, propyl, isopropyl, butyl, isobutyl, sec-butyl, tert-butyl, pentyl and isomers thereof, hexyl and isomers thereof etc.; propyl, isopropyl, butyl and isobutyl are especially preferred.

In some embodiments, R₄ is an aliphatic moiety (preferably an alkyl group) having 6-16 non-hydrogen atoms, preferably these are carbon atoms but oxygen, nitrogen or sulphur atoms may be incorporated, and the moiety may be linear, branched or cyclic.

In some preferred embodiments, R₄ is an isopropyl group.

Of the R₄ groups which comprise a cyclic group, preferred are molecules in which R₄ is cyclohexyl or cyclopentyl.

Suitable non-genetically coded amino acids and modified amino acids which can provide a cationic amino acid include analogues of lysine, arginine and histidine such as homolysine, ornithine, diaminobutyric acid, diaminopimelic acid, diaminopropionic acid and homoarginine as well as trimethylysine and trimethylornithine, 4-aminopiperidine-4-carboxylic acid, 4-amino-1-carbamimidoylpiperidine-4-carboxylic acid and 4-guanidinophenylalanine.

The large lipophilic R group of the AA may contain hetero atoms such as O, N or S, typically there is no more than one heteroatom, preferably it is nitrogen. This R group will preferably have no more than 2 polar groups, more preferably none or one, most preferably none.

The compounds, which are preferably peptides, are preferably of formula (II)

AA₁-AA₂-AA₁-X—Y (II)
wherein:
AA₁ is a cationic amino acid, preferably lysine or arginine but may be histidine or any non-genetically coded or modified amino acid carrying a positive charge at pH 7.0;
AA₂ is an amino acid with a large lipophilic R group, the R group having 14-27 non-hydrogen atoms and preferably containing 2 or more, e.g. 2 or 3, cyclic groups which may be fused or connected, these cyclic groups will typically comprise 5 or 6 non-hydrogen atoms, preferably 6 non-hydrogen atoms; and
X and Y are as defined above.

Further preferred compounds include compounds of formulae (III) and (IV):

AA₂-AA₁-AA₁-X—Y (III)

AA₁-AA₁-AA₂-X—Y (IV)
wherein AA₁, AA₂, X and Y are as defined above. Molecules of formula (II) are more preferred.

From amongst the above compounds certain are particularly preferred. In particular, compounds wherein the amino acid with a large lipophilic R group, conveniently referred to herein as AA₂, is tributyl tryptophan (Tbt) or a biphenylalanine derivative such as Phe(4-(2-Naphthyl)) [also referred to herein as Bip (4-(2-Naphthyl)], Phe(4-(1-Naphthyl)) [also referred to herein as Bip (4-(1-Naphthyl)], Bip (4-n-Bu), Bip (4-Ph) or Bip (4-T-Bu); Phe(4-(2-Naphthyl)) and Tbt being most preferred. In some preferred embodiments, the amino acid with a large lipophilic R group is tributyl tryptophan (Tbt).

Another preferred group of compounds are those wherein Y is —R₁—R₂—R₃ as defined above, preferably wherein R₁ and R₂ are unsubstituted, most preferably wherein R₁ and R₂ are both carbon atoms.

A further preferred group of compounds are those in which —X—Y together is the group —NHCH₂CH₂Ph.

The compounds include all enantiomeric forms, both D and L amino acids and enantiomers resulting from chiral centers within the amino acid R groups and the C-terminal capping group “—X—Y”. β and γ amino acids as well as a amino acids are included within the term ‘amino acids’, as are N-substituted glycines which may all be considered AA units. The molecules of the invention include beta peptides and depsipeptides.

Most preferred compounds are the following:

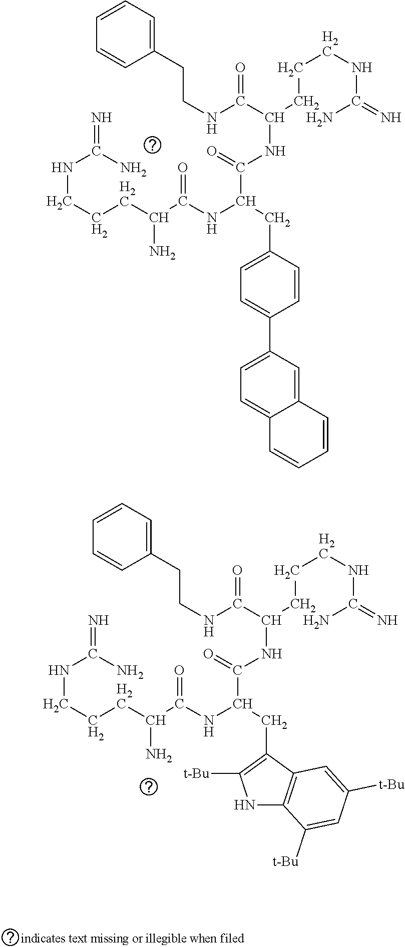 [SMILES: N=C(N)NCCCC(N)C(=O)NC(Cc1ccc(-c2ccc3ccccc3c2)cc1)C(=O)NC(CCCNC(=N)N)C(=O)NCCc1ccccc1 ; CC(C)(C)c1cc(C(C)(C)C)c2[nH]c(C(C)(C)C)c(CC(NC(=O)C(N)CCCNC(=N)N)C(=O)NC(CCCNC(=N)N)C(=O)NCCc3ccccc3)c2c1]

t-Bu represents a tertiary butyl group. This second compound incorporating the amino acid 2,5,7-Tris-tert-butyl-L-tryptophan is the most preferred compound of use in the present invention (and is also referred to herein as AMC-109). Analogues of this compound incorporating other cationic residues in place of Arg, in particular Lys, are also highly preferred. Analogues incorporating alternative C terminal capping groups as defined above are also highly preferred.

A further preferred group of compounds are those in which —X—Y together is selected from the group consisting of —NHCH(CH₃)₂, —NH(CH₂)₅CH₃, —NH(CH₂)₃CH₃, —NH(CH₂)₂CH₃, —NHCH₂CH(CH₃)₂, —NHcyclohexyl and —NHcyclopentyl, particularly preferred are compounds in which —X—Y is the group —NHCH(CH₃)₂ or —NH(CH₂)₅CH₃. A particularly preferred group of compounds are those in which —X—Y together is NHCH(CH₃)₂.

A preferred compound is a compound in which AA₁ is arginine, AA₂ is tributyl tryptophan and —X—Y together is NHCH(CH₃)₂.

Compounds of use in the present invention are preferably peptides.

The compounds of formulae (I) to (IV) may be peptidomimetics and peptidomimetics of the peptides described and defined herein also represent compounds of use in accordance with the present invention. A peptidomimetic is typically characterised by retaining the polarity, three dimensional size and functionality (bioactivity) of its peptide equivalent but wherein the peptide bonds have been replaced, often by more stable linkages. By ‘stable’ is meant more resistant to enzymatic degradation by hydrolytic enzymes. Generally, the bond which replaces the amide bond (amide bond surrogate) conserves many of the properties of the amide bond, e.g. conformation, steric bulk, electrostatic character, possibility for hydrogen bonding etc. Chapter 14 of “Drug Design and Development”, Krogsgaard, Larsen, Liljefors and Madsen (Eds) 1996, Horwood Acad. Pub provides a general discussion of techniques for the design and synthesis of peptidomimetics. In the present case, where the molecule is reacting with a membrane rather than the specific active site of an enzyme, some of the problems described of exactly mimicking affinity and efficacy or substrate function are not relevant and a peptidomimetic can be readily prepared based on a given peptide structure or a motif of required functional groups. Suitable amide bond surrogates include the following groups: N-alkylation (Schmidt, R. et al., Int. J. Peptide Protein Res., 1995, 46, 47), retro-inverse amide (Chorev, M and Goodman, M., Acc. Chem. Res, 1993, 26, 266), thioamide (Sherman D. B. and Spatola, A. F. J. Am. Chem. Soc., 1990, 112, 433), thioester, phosphonate, ketomethylene (Hoffman, R. V. and Kim, H. O. J. Org. Chem., 1995, 60, 5107), hydroxymethylene, fluorovinyl (Allmendinger, T. et al., Tetrahydron Lett., 1990, 31, 7297), vinyl, methyleneamino (Sasaki, Y and Abe, J. Chem. Pharm. Bull. 1997 45, 13), methylenethio (Spatola, A. F., Methods Neurosci, 1993, 13, 19), alkane (Lavielle, S. et. al., Int. J. Peptide Protein Res., 1993, 42, 270) and sulfonamido (Luisi, G. et al. Tetrahedron Lett. 1993, 34, 2391).

The peptidomimetic compounds of the present invention will typically have 3 identifiable sub-units which are approximately equivalent in size and function to amino acids (AA units). The term ‘amino acid’ may thus conveniently be used herein to refer to the equivalent sub-unit of a peptidomimetic compound. Moreover, peptidomimetics may have groups equivalent to the R groups of amino acids and discussion herein of suitable R groups and of N and C terminal modifying groups applies, mutatis mutandis, to peptidomimetic compounds.

As is discussed in the text book referenced above, as well as replacement of amide bonds, peptidomimetics may involve the replacement of larger structural moieties with di- or tripeptidomimetic structures and in this case, mimetic moieties involving the peptide bond, such as azole-derived mimetics may be used as dipeptide replacements. Peptidomimetics and thus peptidomimetic backbones wherein the amide bonds have been replaced as discussed above are, however, preferred.

Suitable peptidomimetics include reduced peptides where the amide bond has been reduced to a methylene amine by treatment with a reducing agent e.g. borane or a hydride reagent such as lithium aluminium-hydride. Such a reduction has the added advantage of increasing the overall cationicity of the molecule.

Other peptidomimetics include peptoids formed, for example, by the stepwise synthesis of amide-functionalised polyglycines. Some peptidomimetic backbones will be readily available from their peptide precursors, such as peptides which have been permethylated, suitable methods are described by Ostresh, J. M. et al. in Proc. Natl. Acad. Sci. USA (1994) 91, 11138-11142. Strongly basic conditions will favour N-methylation over O-methylation and result in methylation of some or all of the nitrogen atoms in the peptide bonds and the N-terminal nitrogen.

Preferred peptidomimetic backbones include polyesters, polyamines and derivatives thereof as well as substituted alkanes and alkenes. The peptidomimetics will preferably have N and C termini which may be modified as discussed herein.

Compounds (e.g. peptides) of use in accordance with the present invention exhibit antimicrobial activity (typically antibacterial activity), in particular they exert a cytotoxic effect through a direct membrane-affecting mechanism and can be termed membrane acting antimicrobial agents. These compounds are lytic, destabilising or even perforating the cell membrane. This offers a distinct therapeutic advantage over agents which act on or intereact with proteinaceous components of the target cells, e.g. cell surface receptors. While mutations may result in new forms of the target proteins leading to antibiotic resistance, it is much less likely that radical changes to the lipid membranes could occur to prevent the cytotoxic effect. The lytic effect causes very rapid cell death and thus has the advantage of killing bacteria before they have a chance to multiply. In addition, the molecules may have other useful properties which kill or harm the target microbes e.g. an ability to inhibit protein synthesis, thus they may have multi-target activity. Antimicrobial activity may be readily determined by any suitable method, and the skilled person is familiar with such methods, e.g. methods described in the Example section herein.

The compounds for use in the invention may be synthesised in any convenient way. Generally the reactive groups present (for example amino, thiol and/or carboxyl) will be protected during overall synthesis. The final step in the synthesis will thus be the deprotection of a protected derivative of the invention.

In building up a peptide, one can in principle start either at the C-terminal or the N-terminal although the C-terminal starting procedure is preferred.

Methods of peptide synthesis are well known in the art but for the present invention it may be particularly convenient to carry out the synthesis on a solid phase support, such supports being well known in the art.

A wide choice of protecting groups for amino acids are known and suitable amine protecting groups may include carbobenzoxy (also designated Z) t-butoxycarbonyl (also designated Boc), 4-methoxy-2,3,6-trimethylbenzene sulphonyl (Mtr) and 9-fluorenylmethoxy-carbonyl (also designated Fmoc). It will be appreciated that when the peptide is built up from the C-terminal end, an amine-protecting group will be present on the a-amino group of each new residue added and will need to be removed selectively prior to the next coupling step.

Carboxyl protecting groups which may, for example be employed include readily cleaved ester groups such as benzyl (Bzl), p-nitrobenzyl (ONb), pentachlorophenyl (OPClP), pentafluorophenyl (OPfp) or t-butyl (OtBu) groups as well as the coupling groups on solid supports, for example methyl groups linked to polystyrene.

Thiol protecting groups include p-methoxybenzyl (Mob), trityl (Trt) and acetamidomethyl (Acm).

A wide range of procedures exists for removing amine- and carboxyl-protecting groups. These must, however, be consistent with the synthetic strategy employed. The side chain protecting groups must be stable to the conditions used to remove the temporary a-amino protecting group prior to the next coupling step.

Amine protecting groups such as Boc and carboxyl protecting groups such as tBu may be removed simultaneously by acid treatment, for example with trifluoroacetic acid. Thiol protecting groups such as Trt may be removed selectively using an oxidation agent such as iodine.

In some embodiments, compounds (e.g. peptides) for use in in accordance with the present invention are encapsulated (e.g. nano-encapsulated or micro-encapsulated). Encapsulation of compounds may be considered the packaging of compounds within a second material (which may be referred to as a matrix or a shell) to form capsules (e.g. nano-capsules). The skilled person is familiar with methods and materials for the encapsulation (e.g. nano-encapsulation) of compounds (e.g. the encapsulation of peptides).

As indicated above, in one aspect antimicrobial articles in accordance with the present invention comprise polyurethane impregnated with a compound of Formula (I). Thus, in this aspect, the polyurethane may be considered to be the substrate (or material) into which a compound of Formula (I) is impregnated.

For the avoidance of doubt, impregnation does not involve a covalent attachment between the polyurethane and a compound of Formula (I), but rather impregnation is a non-covalent, releasable, association between the polyurethane and a compound of Formula (I). Thus, the compound may be considered to be releasably associated with the polyurethane.

Thus, the compound of Formula (I) is capable of being released from (or leaching from or diffusing out of) the polyurethane (or polyurethane substrate) into which it is impregnated (e.g. when brought into contact with moisture, for example at a wound). This is important in the context of the present invention as compounds of Formula (I) have antimicrobial activity, and it is desirable that, in use (e.g. when in contact a wound), the compounds are capable of being released from the polyurethane of the article to an area that requires an antimicrobial activity, for example to prevent or treat the infection of a wound.

Preferably, in use, there is controlled (i.e. sustained) release of the compound of Formula (I) from the polyurethane (or polyurethane substrate). For example, there may be release of a therapeutically effective amount of the compound for at least 2, 3, 4 or 5 days. In some embodiments, there may be sustained release of the compound (preferably a therapeutically effective amount of the compound) for at least 6 hours, 12 hours, 1 day, 2 days, 3 days, 4 days, 5 days, 10 days, 12 days, 15 days, 18 days, 20 days, 25 days or 30 days. In some embodiments, there may be sustained release of the compound (preferably a therapeutically effective amount of the compound) for 1 to 5, 1 to 10, 1 to 15, 1 to 18, 1 to 20, 1 to 25 or 1 to 30 days. In some embodiments, there may be sustained release of the compound (preferably a therapeutically effective amount of the compound) for 2 to 5, 2 to 10, 2 to 15, 2 to 18, 2 to 20, 2 to 25 or 2 to 30 days. In some embodiments, there may be sustained release of the compound (preferably a therapeutically effective amount of the compound) for 5 to 10, 5 to 15, 5 to 18, 5 to 20, 5 to 25 or 5 to 30 days.

A therapeutically effective amount will preferably result in delivery to the local environment of a concentration of the compound which is in excess of the Minimum Inhibitory Concentration (MIC) of the compound for the target bacteria.

As indicated above, preferably, in use, there is sustained release of the compound of Formula (I) from the polyurethane (or polyurethane substrate). Accordingly, preferably, for an antimicrobial article (e.g. a medical device) in accordance with the invention or for a coating in accordance with the invention, in use (e.g. when contacted with water or moisture or an aqueous solution or a body fluid), there is sustained release of the compound of Formula (I) from the polyurethane (or polyurethane substrate). For example, for a medical device in accordance with the invention, in use (e.g. when used in vivo in a subject, or when brought into contact with a subject (e.g. a wound of a subject), or when implanted into a subject), there is preferably sustained release of the compound of Formula (I) from the polyurethane (or polyurethane substrate).

The ability of an impregnated compound to be released from the polyurethane may be readily determined by any suitable method, and the skilled person is familiar with such methods. For example, an antimicrobial article (or antimicrobial coating) impregnated with a compound in accordance with the present invention can be brought into contact with an agar plate that has been inoculated with bacteria (e.g. a bacteria of the genus Staphylococcus) and, after an appropriate incubation time (e.g. 16 hours for 37° C.), the plates can be inspected for the presence of a “zone of inhibition” (i.e. a zone with no bacterial growth or with reduced bacterial growth) around the antimicrobial article. The presence of a “zone of inhibition” (e.g. as compared to a test with control article or coating that is not impregnated with an antimicrobial compound) is indicative that compound can be released from the polyurethane. An exemplary method is described in the Example section herein. An “extraction” test, e.g. as described in the Example section herein, may also be used to assess the ability of an impregnated compound to be released from the polyurethane.

Polyurethanes with any suitable physical properties may be employed in accordance with the present invention. Typically of course, the physical properties should be such that the polyurethane is capable of releasing a compound of Formula (I) (with which it is impregnated) as discussed elsewhere herein.

Alternatively viewed, a compound of Formula (I) may be considered to be dispersed (releasably dispersed) through (or dispersed at least partially through) the polyurethane (polyurethane substrate).

In some embodiments, a compound of Formula (I) may be homogeneously dispersed (homogenously and releasably dispersed) through the polyurethane (polyurethane substrate).

Polyurethanes (which may conveniently also be referred to as PU) are polymers formed by a reaction between alcohols with two or more reactive hydroxyl groups (—OH) per molecule (e.g. diols, triols or other polyols) and isocyanates that have more than one reactive isocyanate group (—NCO) per molecule (e.g. di- or tri-isocyanates or other polyisocyanates). The reaction between such alcohols and isocyanates leads to urethane linkages in the backbone. Typically, polyurethane polymers are formed by the reaction of a di- or tri-isocyanate with a polyol (e.g. a diol such as polyethylene glycol (PEG) or polytetrahydrofuran). Preferred polyols are diols. Preferred isocyanates are diisocyanates. In some cases, polyurethanes may further comprise one or more chain extenders (i.e. one or more chain extenders may also be used in the production of PU).

Many types of polyurethane are known in the art and the skilled person is familiar with these and methods of producing polyurethanes. In particular, antimicrobial articles (e.g. wound dressings) comprising polyurethane are well-known in the art and again the skilled person is familiar with such articles and methods for their manufacture.

In some embodiments, the polyurethane is a polyether polyurethane, e.g. a polyethylene glycol (PEG)-based polyurethane. Thus, in some embodiments, the polyurethane is a polyurethane formed by the reaction between a polyether alcohol (a polyether polyol, preferably a polyether diol)) and an isocyanate, e.g. the reaction between PEG and an isocyanate (preferably a diisocyanate) or the reaction between polytetrahydrofuran and an isocyanate (preferably a diisocyanate).

In some embodiments, the polyurethane is a PEG-analog-based polyurethane. By way of example, in some embodiments, the polyurethane is a polytetrahydrofuran (PTHF)-based polyurethane. Polytetrahydrofuran may also be referred to as poly(tetramethylene oxide) or poly(tetramethylene ether) glycol or polytetramethylene ether glycol (PTMEG).

Typically and preferably in accordance with the present invention, the PU (e.g. a thermoplastic polyurethane; TPU) is such that it is capable of absorbing water (or other aqueous solution or body fluid etc.). Alternatively viewed, in some embodiments, the PU is capable of taking-up water (or other aqueous solution or body fluid, etc.) or can swell when contacted with water (or other aqueous solution or body fluid, etc.).

Typically and preferably in accordance with the present invention, polyol- (preferably diol-) derived parts (or monomers) or moieties) of (or in) the PU (e.g. TPU) are such that they allow the PU to absorb water (or other aqueous solution or body fluid etc.). Alternatively viewed, in some embodiments polyol- (preferably diol-) derived parts (or monomers or moieties) of the PU are capable of taking-up water (or other aqueous solution or body fluid, etc.) or can swell when contacted with water (or other aqueous solution or body fluid, etc.).

Thus, in some embodiments, the PU (e.g. TPU) comprises polyol- (preferably diol-) derived parts (or monomers or moieties) that allow the PU to absorb (or take-up) water (or other aqueous solution or body fluid etc.). Such polyol- (preferably diol-) derived parts (or monomers or moieties) may be considered hydrophilic. Polyol- (preferably diol-) derived parts that allow the PU to absorb (or take-up) water (or other aqueous solution or body fluid etc.) may sometimes be considered as “soft domains”. The term “soft domain” is used to contrast with “hard domains” that PUs may also possess. The term “hard domain” is typically used in reference to isocyanate derived parts (or domains or moieties) of PUs. Soft domains and hard domains may also be referred to as soft segments and hard segments, respectively.

Without wishing to be bound by theory, once water penetrates into a PU (PU substrate), the compound of Formula (I) in accordance with the present invention that is impregnated in (or dispersed in or entrained in) the part of the PU that has been accessed by the water can be dissolved and released.

Typically and preferably, polyols in accordance with the present invention are polymeric polyols (e.g. polymeric diols). Thus, in some preferred embodiments, the PU (e.g. TPU) comprises polymeric polyol- (preferably polymeric diol-) derived parts (or monomers or moieties). Alternatively viewed, in some preferred embodiments, the PU (e.g. TPU) is (or has been) formed (or synthesized or produced or obtained) by the reaction of a polymeric polyol (preferably a polymeric diol) with an isocyanate (preferably a diisocyanate). Optionally a chain extender may also be included, i.e. the reaction may further comprise a chain extender.

In some embodiments, polyether polyols (preferably polyether diols) are preferred. Thus, in some preferred embodiments, the PU (e.g. TPU) comprises polyether polyol- (preferably diol-) derived parts (or monomers or moieties). Alternatively viewed, in some preferred embodiments, the PU (e.g. TPU) is (or has been) formed (or synthesized or produced or obtained) by the reaction of a polyether polyol (preferably a polyether diol) with an isocyanate (preferably a diisocyanate). Optionally a chain extender may also be included, i.e. the reaction may further comprise a chain extender.

Without wishing to be bound by theory, it is believed that PUs formed using polyether polyols (preferably polyether diols) are particularly useful in the context of the present invention as they are hydrophilic, allowing the PUs absorb water. It is believed that this is due to the presence of oxygen atoms.

In some embodiments, PUs comprising soft domains derived from (or based on) polyether polyols (preferably polyether diols) are preferred.

In some preferred embodiments, a polyether polyol in accordance with the present invention is a polyether polyol (more specifically a polyether diol) of Formula (V):

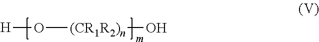

wherein “n” is 2, 3 or 4 (preferably 2 or 4);
wherein each of R₁ and R₂ is independently selected from the group consisting of H or C₁-C₆ alkyl (preferably each of R₁ and R₂ is H); and
wherein “m” is at least 2.

In some embodiments, “m” is 2 to 500, for example 2-250, 2-100, 2-50, 2-10, 5-500, 5-250, 5-100, 5-50, 5-10, 10-500, 10-250, 10-100 or 10-50. In some embodiments, “m” is about 5-75, 5-50, 5-40, 5-30 or 5-20. In some embodiments, “m” is about 10-75, 10-50, 10-40, 10-30 or 10-20. In some embodiments, “m” is about 15-75, 15-50, 15-40, 15-30 or 15-20. In some embodiments, “m” is about 20-75, 20-50, 20-40, 20-30. In some embodiments, “m” is about 5-150 or about 5 to 140 or about 6 to 150 or about 6 to 140. In some embodiments, “m” is about 15-40.

In some preferred embodiments, “n” is 2.

In some preferred embodiments, “n” is 4.

In some preferred embodiments, “n” is 2 or 4, and each of R₁ and R₂ is H.

In some preferred embodiments in which “n” is 2 or 4, “m” is a one of the ranges of numbers as set out above.

In some preferred embodiments, “n” is 2 or 4, each of R₁ and R₂ is H, and “m” is a one of the ranges of numbers as set out above.

In some embodiments, the PU (e.g. TPU) is (or has been) formed (or synthesized or produced) by the reaction of a polyether polyol of Formula (V) with an isocyanate (preferably a diisocyanate).

Alternatively viewed, in some embodiments, the PU (e.g. TPU) comprises polyether polyol- (preferably diol-) derived parts (or monomers or moieties or domains) of Formula (VI):

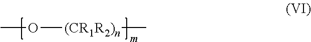

wherein each of R₁, R₂, “n” and “m” are as described above in connection with Formula (V).

Alternatively viewed, in some embodiments, the PU (e.g. TPU) comprises monomers (or moieties) of Formula (VI).

In some embodiments, polyols in accordance with the present invention (e.g. poly(tetramethylene ether) glycol or other polyols in accordance with Formula (V)) may have a molecular weight of between about 500 and about 10,000.

In some embodiments, the PU is (or has been) formed by the reaction of a polyalkylene glycol (for example a polyalkylene glycol having the general formula of formula (V) above) with an isocyanate that has more than one reactive isocyanate group (preferably a diisocyanate). Optionally a chain extender may also be included.

In some embodiments, the isocyanate (preferably a diisocyanate) is an aliphatic isocyanate (preferably an aliphatic diisocyanate). For example, the aliphatic diisocyanate may be aliphatic hydrogenated 4,4′-methylenediphenyl diisocyanate. Aliphatic hydrogenated 4,4′-methylenediphenyl diisocyanate may also be referred to as dicyclohexyl methane diisocyanate or HMDI.

In some other embodiments, the isocyanate (preferably a diisocyanate) is an aromatic isocyanate (preferably an aromatic diisocyanate). For example, the aromatic diisocyanate may be aromatic 4,4′methylenediphenyl diisocyanate.

In preferred embodiments, the isocyanate is an aliphatic isocyanate (preferably an aliphatic diisocyanate).

Thus, in some preferred embodiments, the PU (e.g. TPU) is (or has been) formed (or synthesized) by the reaction of a polyol (preferably a polyether polyol, e.g. a polyether diol, for example in accordance with Formula (V) above) with an aliphatic isocyanate (preferably an aliphatic diisocyanate). Optionally a chain extender may also be included (e.g. 1,4-butane diol).

In certain other embodiments, the PU (e.g. TPU) is (or has been) formed (or synthesized) by the reaction of a polyol (preferably a polyether polyol, e.g. a polyether diol, for example in accordance with Formula (V) above) with an aromatic isocyanate (preferably an aromatic diisocyanate). Optionally a chain extender may also be included (e.g. 1,4-butane diol).

In some embodiments, the PU (e.g. TPU) is (or has been) formed (or synthesized) by the reaction of poly(tetramethylene ether) glycol with an aliphatic isocyanate (preferably an aliphatic diisocyanate). In some embodiments, the PU (e.g. TPU) is (or has been) formed (or synthesized) by the reaction of poly(tetramethylene ether) glycol (e.g. in accordance with Formula (V) above) with dicyclohexyl methane diisocyanate. Optionally a chain extender may also be included (e.g. 1,4-butane diol), i.e. the reaction may further comprise a chain extender.

In some embodiments, the PU (e.g. TPU) is (or has been) formed (or synthesized) by the reaction of poly(tetramethylene ether) glycol with an aromatic isocyanate (preferably an aromatic diisocyanate). In some embodiments, the PU (e.g. TPU) is (or has been) formed (or synthesized) by the reaction of poly(tetramethylene ether) glycol (e.g. in accordance with Formula (V) above) with aromatic 4,4′methylenediphenyl diisocyanate. Optionally, a chain extender may also be included (e.g. 1,4 butane diol), i.e. the reaction may further comprise a chain extender.

As is evident from the above discussion, in some embodiments, polyether polyols (i.e. polyether polyol based PUs) are preferred in accordance with the present invention. However, in some other embodiments, polyester polyols (i.e. polyester polyol based PUs) may be employed. Thus, in some embodiments, the PU (e.g. TPU) comprises polyester polyol- (preferably diol-) derived parts (or monomers or moieties). Alternatively viewed, in some preferred embodiments, the PU (e.g. TPU) is (or has been) formed (or synthesized or produced or obtained) by the reaction of a polyester polyol (preferably a polyester diol) with an isocyanate (preferably a diisocyanate). Preferred isocyanates are discussed elsewhere herein. Optionally, a chain extender may also be included, i.e. the reaction may further comprise a chain extender.

As is evident from the above discussion, polyurethanes may further comprise chain extenders (i.e. in addition to the isocyanate and polyol derived parts). The skilled person in this technical field is familiar with chain extenders. Chain extenders are typically low molecular weight (or short chain) diols or diamines that react with isocyanates (e.g. diisocyanates) to build polyurethane molecular weight and increase block length of hard segments (or hard domains). Chain extenders may be aliphatic or aromatic. The skilled person is familiar is suitable chain extenders, which include, for example, 1,4 butane diol (BDO).

In some embodiments, the polyurethane is a thermoplastic polyurethane. Thermoplastic polyurethanes may be referred to by the acronym TPU. In some preferred embodiments the TPU is a medical grade TPU. In some embodiments, the TPU is an aliphatic TPU. In some embodiments, the TPU is an aliphatic polyether-based TPU. In some embodiments, the TPU is Tecoflex (e.g. Tecoflex EG 80A) (Lubrizol Advanced Materials, Inc.), or is a TPU analogous to (e.g. having one or more of the characteristics of) Tecoflex (e.g. Tecoflex EG 80A). In some embodiments, the TPU is an aromatic TPU. In some embodiments, the TPU is an aromatic copolyester-based TPU. In some embodiments, the TPU is an aromatic polycaprolactone copolyester-based TPU. In some embodiments, the TPU is Pearlcoat DIPP 119 (Lubrizol Advanced Materials, Inc.), or is a TPU analogous to (e.g. having one or more of the characteristics of) Pearlcoat DIPP 119. In some embodiments, the TPU is an aromatic polyether-based TPU. In some embodiments the TPU is Estane 58300 (Lubrizol Advanced Materials, Inc.), or is a TPU analogous to (e.g. having one or more of the characteristics of) Estane 58300.

In some embodiments, the polyurethane is polyurethane of the type used in the Biatain® range of products (Coloplast) or is Pellethane™ (e.g. Pellethane 80A, Lubrizol Advanced Materials, Inc), or is a polyurethane analogous to (e.g. having one or more of the characteristics of) such polyurethanes.

Polyurethanes in accordance with the present invention may be absorbent, e.g. moisture absorbent for example for example capable of absorbing exudates such as wound exudates.

Absorbent polyurethanes are typically preferred for wound dressings (e.g. wound dressings where absorption of exudates is desirable). Absorbent polyurethanes may swell upon contact with moisture such as wound exudates. Thus, the polyurethane in accordance with the present invention may be hygroscopic. The polyurethane in accordance with the present invention may be hydrophilic.

Preferably, the polyurethane in accordance with the present invention is a medical-grade polyurethane. Thus, preferably, the polyurethane in accordance with the present invention is biocompatible.

The polyurethane in accordance with the present invention may be in the form of a polyurethane foam. Thus, in some embodiments, the invention provides an antimicrobial article comprising a polyurethane foam impregnated with a compound of Formula (I) as defined herein.

Polyurethane foams in accordance with the present invention have an open cell structure. Thus, the polyurethane foams may be considered permeable (or semi-permeable) or porous polyurethanes. In accordance with the present invention, such polyurethane foams are impregnated with a compound of Formula (I). Such polyurethane foams are capable of releasing (e.g. via sustained release) a compound of Formula (I), for example when brought into contact with moisture (e.g. at a wound such as an exuding wound). Typically and preferably, polyurethane foams are also capable of absorbing moisture, e.g. wound exudate.

The skilled person is familiar with polyurethane foams, including polyurethane foams suitable for antimicrobial articles (e.g. polyurethane foam dressings).

Polyurethane foams with any suitable physical properties may be employed in accordance with the present invention, for example a polyurethane foam with any pore size, density, thickness, moisture vapour transmission rate (MVTR) or absorptive capacity may be employed. Typically of course, the physical properties should be such that the polyurethane foam is capable of releasing a compound of Formula (I) (with which it is impregnated) as discussed elsewhere herein and absorbing moisture (e.g. wound exudate).

In some embodiments, the average (e.g. mean) pore size diameter in the polyurethane foam is 10 μm to 1000 μm, for example 10 μm to 900 μm, 10 μm to 800 μm, 10 μm to 700 μm, 10 μm to 600 μm, 10 μm to 500 μm, 10 μm to 400 μm, 10 μm to 300 μm, 10 μm to 200 μm or 10 μm to 100 μm. In some embodiments, the average (e.g. mean) pore size diameter in the polyurethane foam is 20 μm to 1000 μm, for example 20 μm to 900 μm, 20 μm to 800 μm, 20 μm to 700 μm, 20 μm to 600 μm, 20 μm to 500 μm, 20 μm to 400 μm, 20 μm to 300 μm, 20 μm to 200 μm or 20 μm to 100 μm. In some embodiments, the average (e.g. mean) pore size diameter in the polyurethane foam is 100 μm to 1000 μm, for example 100 μm to 900 μm, 100 μm to 800 μm, 100 μm to 700 μm, 100 μm to 600 μm, 100 μm to 500 μm, 100 μm to 400 μm, 100 μm to 300 μm or 100 μm to 200 μm. In some embodiments, the average pore size diameter of the pores at the surface of the polyurethane foam is smaller than the average pore size diameter in the interior polyurethane foam. Average (e.g. mean) pore size diameter may be as assessed by any suitable means and the skilled person is familiar with suitable methods, e.g. field-emission scanning electron microscopy.

In some embodiments, the polyurethane foam has a uniform (or a relatively uniform) pore size. In other embodiments, the polyurethane foam has pores that are non-uniform in size. The pores may be homogenous (or substantially homogenous) or may be non-homogenous in morphology.

In some embodiments, the polyurethane foam has a density of 0.05 g/cm³ to 0.5 g/cm³, 0.05 g/cm³ to 0.4 g/cm³, 0.05 g/cm³ to 0.3 g/cm³, 0.05 g/cm³ to 0.2 g/cm³ or 0.05 g/cm³ to 0.1 g/cm³. In some embodiments, the polyurethane foam has a density of 0.1 g/cm³ to 0.5 g/cm³, 0.1 g/cm³ to 0.4 g/cm³, 0.1 g/cm³ to 0.3 g/cm³ or 0.1 g/cm³ to 0.2 g/cm³.

In some embodiments, the polyurethane foam has a thickness of 1 mm to 20 mm, 1 mm to 10 mm, 1 mm to 5 mm, 1 mm to 4 mm, 1 mm to 3 mm, 1 mm to 2 mm. In some embodiments, the polyurethane foam has a thickness of 2 mm to 20 mm, 2 mm to 10 mm, 2 mm to 5 mm, 2 mm to 4 mm or 2 mm to 3 mm. In some embodiments, the polyurethane foam has a thickness of 3 mm to 20 mm, 3 mm to 10 mm, 3 mm to 5 mm or 3 mm to 4 mm. In some embodiments, the polyurethane foam has a thickness of 4 mm to 20 mm, 4 mm to 10 mm or 4 mm to 5 mm. In some embodiments, the polyurethane foam has a thickness of 5 mm to 20 mm or 5 mm to 10 mm. The thickness of the foam may be as assessed by any suitable means and the skilled person is familiar with suitable methods, e.g. by using a micrometer.

In some embodiments, the polyurethane foam has moisture vapour transmission rate (MVTR) of 100 to 3000 g/m²/day, 100 to 2000 g/m²/day, 100 to 1000 g/m²/day or 100 to 500 g/m²/day. In some embodiments, the polyurethane foam has moisture vapour transmission rate (MVTR) of 500 to 3000 g/m²/day or 500 to 2000 g/m²/day or 500 to 1000 g/m²/day. MVTR is a well-known measure of the passage of water vapour through a substance or material. MVTR may be as assessed by any suitable means, and the skilled person is familiar with suitable methods, e.g. by using a thermo-hygrostat.

In some embodiments, the polyurethane foam has an absorption capacity (liquid or moisture absorption capacity e.g. water absorption capacity) of 0.05 g/cm² to 5 g/cm², 0.05 g/cm² to 2 g/cm², 0.05 g/cm² to 1 g/cm². In some embodiments, the polyurethane foam has an absorption capacity of 0.5 g/cm² to 5 g/cm², 0.5 g/cm² to 2 g/cm², 0.5 g/cm² to 1 g/cm². In some embodiments, the polyurethane foam has an absorption capacity of 1 g/cm² to 5 g/cm² or 1 g/cm² to 2 g/cm². Absorption capacity may be as assessed by any suitable means, and the skilled person is familiar with suitable methods, e.g. for example by weighing a sample of polyurethane foam and recording its weight (W1), then immersing said sample of polyurethane foam in an excess of deionized water (e.g. for 30 minutes at 37° C.) and then re-weighing said sample of polyurethane foam and recording its weight (W2), where absorption capacity (g/cm²)=(W2−W1)g/initial area of the polyurethane foam (cm²).

As mentioned above, the skilled person is familiar with polyurethane foams, including polyurethane foams suitable for antimicrobial articles for medical uses (e.g. polyurethane foam dressings) and thus is familiar with methods for producing polyurethane foams for use in accordance with the present invention.

In the production of polyurethane foams, blowing agents are typically used. Blowing agents are substances capable of producing (or introducing) a cellular structure via a foaming process in materials that undergo some degree of hardening or phase transition during their production, such as polymers and plastics. Blowing agents (which are sometimes also known as ‘pneumatogens’) create pores (or holes) are can thus facilitate the production polyurethane with a cellular structure (a polyurethane foam). By way of one example, polyurethane foams may be produced by the addition of water (and optionally an excess of isocyanate) during the polyurethane production process, with the water reacting with isocyanate groups, which releases carbon dioxide that acts as a blowing agent to produce a foamed structure. Such a blowing reaction may be catalysed by amines, such as tertiary amines. A surfactant (e.g. a silicone surfactant) may be used stabilize the foam cells in the polyurethane foaming process. Other methods for producing polyurethane foams are also known and the skilled person is familiar with these and foams produced by any appropriate means may be used in accordance with the present invention.

Although in some embodiments polyurethane (PU) foams represent one preferred form of PU, in other embodiments the polyurethane is not a polyurethane foam (i.e. it is a non-foamed polyurethane). PU foams may be preferred when the antimicrobial article is a wound dressing.

Thus, in some embodiments, the polyurethane is a polyurethane foam.

In other embodiments, the polyurethane is not a foam (i.e. not in the form of a foam). Non-foam PUs may be considered to be solid, or relatively solid, for example in comparison with a PU foam. Although non-foam PUs may be considered solid (or relatively solid), they may still be flexible. Thus, in some embodiments, the PU is solid but flexible.

In some embodiments, the polyurethane is in the form of a sheet, layer, patch or pad (or the like) of polyurethane. Thus in some embodiments, the antimicrobial article comprises a sheet, layer, patch or pad that comprises polyurethane, said sheet, layer, patch or pad being impregnated with a compound of Formula (I).

Impregnation of polyurethane (e.g. a PU foam or a non-foam PU) with a compound of Formula (I) may be achieved by any suitable means.

For example, impregnation of polyurethane may be done by applying a solution of a compound of Formula (I) to the polyurethane (or incubating or soaking polyurethane with a solution of a compound of Formula (I)) and then allowing the polyurethane to dry. After application of the solution of a compound of Formula (I) the polyurethane may swell (or at least the outer/surface layers of the polyurethane may swell) (as it absorbs the solution). After the application (and swelling if swelling occurs), the polyurethane is allowed to dry. Any suitable solvent for the solution of a compound of Formula (I) may be used. In some embodiments, the solution is an aqueous solution of a compound of Formula (I). For example, the solvent for the solution of a compound of Formula (I) may be water (e.g. distilled water). In other embodiments, the solvent may be, or may comprise, an organic solvent, e.g. may comprise ethanol and/or chloroform or may comprise a polar aprotic solvent (e.g. as described elsewhere herein). In some embodiments, the solvent is an ethanol/chloroform mix (e.g. a 2:1 ethanol:chloroform mix). In some embodiments, the solvent is a polar aprotic solvent (or polar aprotic organic solvent). Organic solvents (e.g. chloroform:ethanol solvents or polar aprotic solvents as described elsewhere herein) may be particularly useful in connection with non-foam PU-materials.

Of course, typically a solution of a compound of Formula (I) may be applied to (or incubated with) the polyurethane such that a therapeutically effective (antimicrobially effective) amount of the compound is impregnated into the polyurethane. In some cases, a solution of a compound of Formula (I) may be applied to the polyurethane such that there is at least 0.1 mg, at least 0.5 mg, at least 1 mg, at least 2 mg, at least 5 mg, at least 10 mg or at least 20 mg of the compound impregnated (or applied) per cm² of the polyurethane. In some cases, a solution of a compound of Formula (I) may be applied to the polyurethane such that there is 0.1 mg-20 mg, 0.5 mg-20 mg, 1 mg-20 mg, 2 mg-20 mg, 5 mg-20 mg, 10 mg-20 mg, 0.1 mg-10 mg, 0.5 mg-10 mg, 1 mg-10 mg, 2 mg-10 mg, 5 mg-10 mg, 0.1 mg-5 mg, 0.5 mg-5 mg, 1 mg-5 mg, 2 mg-5 mg, 0.1 mg-5 mg, 0.5 mg-5 mg, 1 mg-5 mg, 2 mg-5 mg, 0.1 mg-2 mg, 0.5 mg-2 mg, 1 mg-2 mg, 0.1 mg-1 mg or 0.5 mg-1 mg of the compound impregnated (or applied) per cm² into the polyurethane. In some cases, a solution of a compound of Formula (I) may be applied to the polyurethane such that there is 0.5 mg of the compound impregnated (or applied) per cm² of the polyurethane.

In some cases, a solution of a compound of Formula (I) may be applied to the polyurethane such that there is at least 0.05 mg, at least 0.1 mg, at least 0.5 mg, at least 1 mg, at least 2 mg, at least 5 mg, at least 10 mg, at least 20 mg, at least 40 mg, at least 50 mg at least 100 mg, or at least 200 mg of the compound impregnated (or applied) per cm³ of the polyurethane. In some cases, a solution of a compound of Formula (I) may be applied to the polyurethane such that there is 0.05-200 mg, 0.1 mg-200 mg, 0.5 mg-200 mg, 1 mg-200 mg, 2 mg-200 mg, 5 mg-200 mg, 10 mg-200 mg, 20 mg-200 mg, 40 mg-200 mg, 50 mg-200 mg, 100 mg-200 mg, 0.05-50 mg, 0.1 mg-50 mg, 0.5 mg-50 mg, 1 mg-50 mg, 2 mg-50 mg, 5 mg-50 mg, 10 mg-50 mg, 20 mg-50 mg, 0.05-10 mg, 0.1 mg-10 mg, 0.5 mg-10 mg, 1 mg-10 mg, 2 mg-10 mg or 5 mg-10 mg of the compound impregnated (or applied) per cm³ of the polyurethane. In some cases, a solution of a compound of Formula (I) may be applied to the polyurethane such that there is 0.5-10 mg, 0.5-5 mg, 0.5-2 mg, 0.5-1 mg, 1-10 mg, 1-5 mg or 1 mg-2 mg of the compound impregnated (or applied) per cm³ of the polyurethane.

In some embodiments, polyurethane in accordance with the invention is impregnated with (or loaded with or applied with) one of the above amounts (or concentrations) of compound.

In some embodiments, the solution of a compound of Formula (I) applied to the polyurethane (which may conveniently be referred to as the swelling agent as the PU may swell as described elsewhere herein) has a concentration of compound of Formula (I) of 0.01%-10%, 0.05%-10%, 0.1%-10%, 0.2%-10%, 0.25%-10%, 0.5%-10%, 1%-10%, 2%-10% or 5%-10%, 0.01%-5%, 0.05%-5%, 0.1%-5%, 0.2%-5%, 0.25%-5%, 0.5%-5%, 1%-5%, 2%-5%, 0.01%-3%, 0.05%-3%, 0.1%-3%, 0.2%-3%, 0.25%-3%, 0.5%-3%, 1%-3%, 2%-3% (e.g. weight/volume %). In some embodiments, the solution of a compound of Formula (I) (the swelling agent) has a concentration of compound of Formula (I) of up to 0.5%, up to 1%, up to 2%, up to 3%, up to 5%, or up to 10% (e.g. weight/volume %). In some embodiments, the solution of a compound of Formula (I) (the swelling agent) has a concentration of compound of Formula (I) of at least 0.01%, at least 0.05%, at least 0.1%, at least 0.2%, at least 0.25%, at least 0.5%, at least 1%, at least 2%, at least 3%, or at least 5% (e.g. weight/volume %). In some embodiments, the solution of a compound of Formula (I) (the swelling agent) has a concentration of compound of Formula (I) of about 1%, about 2%, about 3%, about 4%, about 5% or about 10% (e.g. weight/volume %). By way of example, the solution of a compound of Formula (I) (the swelling agent) may have a concentration of compound of Formula (I) of 2.8% (w/v).

In some embodiments, impregnation of a PU foam with a compound of formula (I) may be done by applying a solution (e.g. an aqueous solution e.g. the solvent may be water) of a compound of Formula (I) to the PU foam (or incubating or soaking PU foam with such a solution) and then allowing the polyurethane to dry. Preferred features of impregnation methods discussed elsewhere herein may be applied to PU foams.

In some embodiments, impregnation of PU that is not in the form of foam (i.e. PU that is not a PU foam) with a compound of formula (I) may be done by applying a solution (typically a solution in which the solvent is an organic solvent (or more than one organic solvent), e.g. as described elsewhere herein) of a compound of Formula (I) to the PU (or incubating or soaking the PU with such a solution) and then allowing the polyurethane to dry. In such embodiments, the PU typically swells upon application of (or incubation with) the solution. As described elsewhere herein, the length of time of the application (or incubation), or swelling time, may be chosen based on the nature (e.g. thickness) of the PU to be impregnated and/or the depth to which impregnation of the PU is desired. Preferred features of impregnation methods discussed elsewhere herein may be applied to PU that is not in the form of a foam.

Another method of impregnating polyurethane with a compound of Formula (I) is electrospinning. Electrospinning is a well-known voltage-driven process governed by the electrohydrodynamic phenomena where fibres (e.g. nanofibres) can be made from a polymer solution. To obtain impregnated polyurethane in accordance with the present invention via an electrospinning method, a solution (or “spinning solution”) comprising the polyurethane and a compound of Formula (I) are electrospun into polyurethane fibres impregnated with said compound. As polyurethane fibres are extruded via electrospinning, a compound of Formula (I) associates with the polyurethane fibres and thus becomes impregnated into the electrospun polyurethane fibres (or fibre product, or mesh of PU fibres, or network of PU fibres, or mat of PU fibres) produced. Electrospinning is well-known in the art and the skilled person would be familiar with suitable electrospinning materials and methods.

In some embodiments, the polyurethane in accordance with the present invention is a mesh of polyurethane fibres (or a PU fibre product, or a sheet of PU fibres, or a network of PU fibres, or a mat of PU fibres or the like). In some such embodiments, the polyurethane fibres (or meshes or mats etc. of PU fibres) are produced by electrospinning. In some embodiments, polyurethane impregnated with a compound of Formula (I) is produced by an electrospinning method (e.g. as discussed elsewhere herein).

Another method of impregnating polyurethane with a compound of Formula (I) is to include said compound in the reaction mixture during the production of the polyurethane polymer, i.e. to include said compound in the reaction mixture together with the alcohol and isocyanate that react with each other to form polyurethane. For the avoidance of doubt, this does not lead to a compound of Formula (I) being covalently attached to the polyurethane polymer produced. Rather, a compound of Formula (I) associates with the polyurethane produced and thus becomes impregnated into the polyurethane as it is produced. In some cases, if the compound of Formula (I) is included in the reaction mixture during the production of the polyurethane polymer, the compound may be included in an encapsulated form (e.g. a nano-encapsulated form or micro-encapsulated form).

Another method of obtaining polyurethane impregnated with a compound of Formula (I) is to provide a composition (liquid composition or solution or formulation) comprising at least one organic solvent (e.g. at least one polar aprotic solvent), a thermoplastic polyurethane (TPU) and a compound of Formula (I), wherein the TPU and a compound of Formula (I) are dissolved in said at least one organic solvent. Such compositions may be conveniently referred to as coating compositions or painting compositions. Such a composition can be used to coat (or paint) an article (or part of an article), and when the solvent then evaporates from the composition (after an article, or part thereof, has been painted/coated with the solution) the article has (i.e. is left with) a coating (or layer or surface layer or paint) comprising polyurethane impregnated with a compound of Formula (I). Preferred features of impregnation methods discussed elsewhere herein may, as appropriate, be applied to aspects or embodiments of the invention relating to painting (or coating) compositions, painting (or coating) methods or articles that have been painted (or coated) with painting (or coating) compositions. Preferred features in relation to painting (or coating) compositions, painting (or coating) methods, and articles that have been painted (or coated) with painting (or coating) compositions are discussed elsewhere herein in connection with other aspects of the invention. Painting/coating methods in accordance with the present invention may be particularly advantageous as a wide range of articles (or parts thereof) can be readily provided with an antimicrobial coating (or layer), simply by “painting” them with a painting/coating composition of the present invention.

The antimicrobial article may be any article that may be susceptible to microbial (typically bacterial) contamination.

Typically, the antimicrobial article is a medical device. Thus, in some embodiments, the antimicrobial article is an article suitable for use in the prevention or treatment of an infection (preferably a bacterial infection) in a medical setting.

In preferred embodiments, the antimicrobial article is a dressing.

In preferred embodiments the antimicrobial article is a wound dressing, surgical pad, anti-scar dressing, or the like.

In particularly preferred embodiments the antimicrobial article is a wound dressing.

In some embodiments, the wound dressing comprises of a sheet (or layer or patch or pad) of polyurethane impregnated with a compound of Formula (I).

In some embodiments, the wound dressing may comprise one or more additional components.

In some embodiments, the sheet (or layer or patch or pad or the like) of polyurethane impregnated with a compound of Formula (I) may be used in combination with other wound dressing components, e.g. gauzes, bandages, secondary dressings etc. For example, in some embodiments, a wound dressing may comprise a sheet (or layer or patch or pad or the like) of polyurethane impregnated with a compound of Formula (I) and one or more additional wound dressing components, for example an additional absorbent layer and/or a secondary layer (or outer layer or cover layer or backing layer) that, for example, may be act to secure the wound dressing to the skin. For example, a polyurethane patch impregnated with a compound of Formula (I) may be provided with an adhesive backing layer to secure the polyurethane patch to the skin.

Thus, in some embodiments, the wound dressing may be a multi-component wound dressing (or multi-layer) wound dressing in which a sheet (or layer or patch or pad or the like) of polyurethane impregnated with a compound of Formula (I) is one component (or one layer).

In some embodiments, the wound dressing consists of a sheet (or layer or patch or pad or the like) of polyurethane (e.g. PU foam) impregnated with a compound of Formula (I).

In some embodiments, the wound dressing consists of a sheet (layer or patch or pad or the like) comprising polyurethane (e.g. PU foam) impregnated with a compound of Formula (I).

The wound may be a partial thickness wound or a full thickness wound. The wound may be a skin tear, abrasion, laceration (cut) or burn (e.g. first or second degree burn). The wound may be an incisional wound. The wound may be an excisional wound. The wound may be a surgical wound.

The wounds may be acute or chronic. Acute wounds are wounds that proceed orderly through the three recognised stages of the healing process (i.e. the inflammatory stage, the proliferative stage and the remodeling phase) without a protracted time course. Chronic wounds, however, are those wounds that do not complete the ordered sequence of biochemical events because the wound has stalled in one of the healing stages. Viewed alternatively a chronic wound is a wound that has not healed within at least 40 days, preferably at least 50 days, more preferably at least 60 days, most preferably at least 70 days. In some embodiments chronic wounds are preferred.

In some embodiments, the wound is an exuding wound (i.e. a wound producing wound exudate or wound fluid).

The wound to be treated may be a breach in, or denudement of, the tissue for instance caused by surgical incision or trauma, e.g., mechanical, thermal, electrical, chemical or radiation trauma; a spontaneously forming lesion such as a skin ulcer (e.g. a venous, diabetic or pressure ulcer); a blister (e.g. a friction or thermal blister or a blister caused by pathogen infection such as chicken pox); an anal fissure or a mouth ulcer.

In other embodiments the antimicrobial article is not a dressing. The antimicrobial article may be another type of medical device. For example, medical devices include surgical fasteners, catheters (e.g. urinary catheters or central venous catheters), lines etc. and implants or prostheses including orthopedic (or joint) implants such as hip and knee implants, as well as dental implants, pins, stents, cardiac rhythm devices, deep brain stimulation devices and intrauterine devices. In some embodiments, “in-dwelling” medical devices are preferred. One particularly preferred type of medical device is a catheter (e.g. a urinary catheter).

In another aspect, the present invention provides polyurethane (e.g.

polyurethane foam, or a polyurethane that is not in the form of a foam (which may be considered a solid PU), or a polyurethane (e.g. TPU) coating) impregnated with a compound of Formula (I). Embodiments of other aspects of the invention described herein apply, mutatis mutandis, to this aspect of the invention.

In another aspect, the present invention provides a method of producing polyurethane impregnated with a compound of Formula (I), said method comprising (i) applying a solution of a compound of Formula (I) to the polyurethane (or incubating the polyurethane with a solution of Formula (I)) and (ii) drying the polyurethane to which said solution has been applied, thereby producing polyurethane impregnated with a compound of Formula (I). The applying of step (i) may lead to a swelling of the polyurethane. Thus, the drying of step (ii) is may be the drying of swollen polyurethane obtained after the application (or incubation) of the solution in step (i). The drying may be done at ambient temperature (i.e. passive drying) or alternatively an active drying step may be performed. In some embodiments, the drying may be done at about 20° C. (e.g. for about 12 hours). Embodiments of other aspects of the invention described herein apply, mutatis mutandis, to this aspect of the invention. For example, preferred compounds of Formula (I) and preferred amounts or concentrations compounds of Formula (I) in the solution are described elsewhere herein. Suitable solvents (e.g. water or ethanol:chloroform or other organic solvents) for a solution of a compound of Formula (I) are also described elsewhere herein.

In some embodiments of methods of producing polyurethane impregnated with a compound of Formula (I) that comprise applying a solution of a compound of Formula (I) to the polyurethane (or incubating the polyurethane with a solution of Formula (I)), that may swell the polyurethane, the solution may be applied to the polyurethane for between 1 minute and 24 hours, between 1 minute and 12 hours, between 1 minute and 10 hours, between 1 minute and 5 hours, between 1 minute and 2 hours, between 1 minute and 1 hour, between 1 minute and 30 minutes, between 1 minute and 10 minutes or between 1 minute and 5 minutes. In some embodiments, the solution may be applied to the polyurethane for between 3 minutes and 24 hours, between 3 minutes and 12 hours, between 3 minutes and 10 hours, between 3 minutes and 5 hours, between 3 minutes and 2 hours, between 3 minutes and 1 hour, between 3 minutes and 30 minutes, between 3 minutes and 10 minutes, or between 3 minutes and 5 minutes.

In some embodiments, the solution may be applied to the polyurethane (or incubated with a solution of Formula (I)) for up to 3 minutes, for up to 5 minutes, for up to 10 minutes, for up to 30 minutes, for up to 1 hour, for up to 5 hours, for up to 10 hours, for up to 12 hours or up to 24 hours. In some embodiments, the solution may be applied to the polyurethane for at least 3 minutes, at least 5 minutes, at least 10 minutes, at least 30 minutes, at least 1 hour, at least 5 hours, at least 10 hours, or at least 12 hours. In some embodiments, the solution may be applied to the polyurethane for about 3 minutes or about 10 hours.

In some embodiments of methods of producing polyurethane impregnated with a compound of Formula (I) that comprise applying a solution of a compound of Formula (I) to the polyurethane (or incubating with a solution if Formula (I)) to swell the polyurethane, the solution may be applied to the polyurethane for a length of time (or swelling time) that achieves an absorption (i.e. a swelling of the polyurethane) of at least 5%, at least 10%, at least 15%, at least 20%, at least 30%, at least 40%, at least 50%, at least 60%, at least 70%, at least 80%, at least 90% or at least 95%. In some embodiments, the solution may be applied to the polyurethane for a length of time that achieves an absorption (i.e. swelling of the polyurethane) of up to 5%, up to 10%, up to 15%, up to 20%, up to 30%, up to 40%, up to 50%, up to 60%, up to 70%, up to 80%, up to 90%, up to 95% or up to 100%. In some embodiments, the solution may be applied to the polyurethane for a length of time that achieves an absorption (i.e. swelling of the polyurethane) of between 5% and 100%, between 10% and 100%, between 20% and 100%, between 30% and 100%, between 40% and 100%, between 50% and 100%, between 60% and 100%, between 70% and 100%, between 80% and 100%, between 90% and 100%, between 5% and 50%, between 10% and 50%, between 20% and 50%, between 30% and 50%, between 40% and 50%, between 5% and 20%, between 10% and 20%, or between 15% and 20%. “% absorption” is the % increase in weight of the polyurethane after the application of the solution of a compound of Formula (I) (i.e. after the swelling) compared to the weight of the polyurethane prior to the application of the solution of a compound of Formula (I) (i.e. before the swelling). Optionally, residual solution on the surface of the polyurethane may be superficially dried (e.g. with cellulose wipes) before the post-application (post-swelling) weighing.

The length of time of the application (swelling time) may be chosen based on the nature (e.g. thickness) of the polyurethane to be impregnated and/or the depth to which impregnation of the polyurethane is desired.

In some embodiments, the present invention provides a method of producing polyurethane foam impregnated with a compound of Formula (I), said method comprising (i) applying a solution of a compound of Formula (I) to the polyurethane foam (or incubating the polyurethane foam with a solution of Formula (I)) and (ii) drying the polyurethane foam to which said solution has been applied, thereby producing polyurethane foam impregnated with a compound of Formula (I). In some such embodiments, the solution may be an aqueous solution (e.g. the solvent may be water), but in other embodiments the solvent may be an organic solvent. Preferred features of impregnation methods discussed elsewhere herein may be applied to methods of producing impregnating PU foams.

Methods of impregnating PU foam typically produce an impregnated PU foam in which a compound of Formula (I) is impregnated (or dispersed) homogenously or uniformly (or substantially homogenously or substantially uniformly) throughout the PU foam (i.e. throughout the PU foam substrate).

In one aspect, the invention provides a PU foam impregnated with a compound of Formula (I) wherein said compound of Formula (I) is impregnated (or dispersed) homogenously or uniformly (or substantially homogenously or substantially uniformly) throughout the PU foam (i.e. throughout the PU foam substrate).

In one aspect, the invention provides a PU foam impregnated with a compound of Formula (I) produced by a method of producing impregnated polyurethane foam in accordance with the invention.

In some embodiments, the invention provides antimicrobial articles (e.g. wound dressings) comprising PU foam impregnated with a compound of Formula (I) produced by a method of producing impregnated polyurethane foam in accordance with the invention.

In some embodiments, the present invention provides a method of producing polyurethane that is not in the form of foam (i.e. PU that is not a PU foam) impregnated with a compound of Formula (I), said method comprising (i) applying a solution (typically a solution in which the solvent is an organic solvent (or more than one organic solvent) e.g. as described elsewhere herein) of a compound of Formula (I) to the PU (or incubating or soaking the PU with such a solution) and (ii) drying the PU to which said solution has been applied, thereby producing polyurethane impregnated with a compound of Formula (I). In such embodiments, the PU (or at least the outer/surface layers of the PU) typically swells upon application of (or incubation with) the solution. As described elsewhere herein, the length of time of the application (or incubation), or swelling time, may be chosen based on the nature (e.g. thickness) of the PU to be impregnated and/or the depth to which impregnation of the PU is desired. Preferred features of impregnation methods discussed elsewhere herein may be applied to methods of producing impregnated PU that is not in the form of a foam. For example, the drying may be done at ambient temperature (i.e. passive drying) or alternatively an active drying step may be performed.

Any appropriate solvent (or combination of solvents) may be used as the solvent(s) in the solution of a compound of Formula (I) to achieve swelling of PU that is not in the form of a foam (or at least the outer/surface layers of the PU) and thus impregnation of compound into the PU. The skilled person could select an appropriate solvent. Typically, the solvent is an organic solvent, for example, an alcohol (e.g. ethanol) and/or chloroform may be used (e.g. a mixture of alcohol and chloroform may be used, e.g. a mixture of ethanol and chloroform). Alternatively, a polar aprotic solvent may be used e.g. as described elsewhere herein.

Methods of impregnating PU that is not in the form of a foam typically produce an impregnated PU in which a compound of Formula (I) is not homogenously impregnated or dispersed throughout the PU (i.e. not homogenously impregnated or dispersed throughout the PU substrate).

Without wishing to be bound by theory, when impregnating PU that is not in the form of a foam (e.g. a solid polyurethane or non-foamed polyurethane), during the swelling process a concentration gradient may be present with the outer layer of the polyurethane (i.e. the polyurethane at, or closest to, to the surface of the PU substrate) being saturated and the inner layers (furthest from the surface) being only partially saturated (or not saturated). By stopping the swelling process before total saturation (or impregnation) of the whole polyurethane sample/article is reached, a polyurethane sample/article may be obtained in which the surface/outer layers contain a desired concentration of compound. The inner layers of the polyurethane (i.e. the polyurethane further/furthest from the surface) may thus contain less compound than at the surface/outer layers (or contain no peptide). Put another way, and again without being bound by theory, by controlling the swelling time the degree to which (or depth to which) the swelling agent (and thus the compound) penetrates the polyurethane can be controlled. Thus, by controlling the swelling time, impregnation of polyurethane can be controlled to achieve, if desired, impregnation of only the surface/outer layers of polyurethane. The thickness of the impregnated portion (or layer) could then determine the leaching rate of the compound and the time-to-depletion of the compound.

Thus, in some embodiments, methods of impregnating PU that is not in the form of a foam produce PU that is impregnated with a compound of Formula (I) only at the outer layer(s) of the PU (or superficial layers of the PU or PU substrate) and that is not impregnated (or is impregnated to a lesser extent) at the inner layer(s) of the PU. Thus, in some embodiments, methods of impregnating PU that is not in the form of a foam may produce PU that is impregnated with a compound of Formula (I) wherein there is a concentration gradient of compound in the PU, with higher concentrations being found in the outer layer(s) of the PU as compared to inner layer(s) of the PU.

Thus, in some embodiments, methods of impregnating PU that is not in the form of a foam produce PU wherein a compound in accordance with Formula (I) is impregnated into said PU in (or over) only a portion of the total depth of the PU (PU substrate) being impregnated. Alternatively viewed, in some embodiments, methods of impregnating PU that is not in the form of a foam produce PU wherein a compound in accordance with Formula (I) is impregnated into said PU in (or over) only a portion of the total depth from the surface of the PU (PU substrate) being impregnated.

In some embodiments, the portion of the total depth (or total depth from the surface of the PU) substrate impregnated with a compound of Formula (I) is ≤90%, ≤80%, ≤70%, ≤60%, ≤50%, ≤40%, ≤30%, ≤20%, ≤10%, ≤5% or ≤2% of the total depth of the PU. Preferably, at least 1%, at least 2%, at least 5%, at least 10%, at least 20%, at least 30%, at least 40% or at least 50% of the total depth of the PU (or total depth from the surface of the PU substrate) is impregnated with a compound of Formula (I). In some embodiments, between 1% and 90% of the total depth of the PU (or total depth from the surface of the PU substrate) is impregnated with a compound of Formula (I), preferably between 1% and 90%, 2% and 90%, 5% and 90%, 10% and 90%, 20% and 90%, 30% and 90%, 40% and 90%, 50% and 90%, 1% and 50%, 2% and 50%, 5% and 50%, 10% and 50%, 20% and 50%, 30% and 50%, 40% and 50%, 1% and 20%, 2% and 20%, 5% and 20%, 10% and 20%, 1% and 10%, 2% and 10%, 5% and 10%, 1% and 5%, or 2% and 5%.

In some embodiments, the swelling time is chosen depending on the nature (e.g. thickness) of the PU to be impregnated and/or the depth to which impregnation of the PU is desired. In some such embodiments, (i) short (or shorter) swelling times may be used when the PU to be impregnated is thin (or relatively thin) and/or impregnation of the inner (or deeper) layer(s) of the PU is not desired, or (ii) long (or longer) swelling times may be used when the PU is thick (or relatively thick) and/or impregnation of the inner (or deeper) layer(s) of the PU is desired. Appropriate swelling times can be determined by the skilled person depending on the desired characteristics of the impregnated PU.

In one aspect, and in some embodiments, the invention provides PU that is not in the form of a foam impregnated with a compound of Formula (I) produced by a method of producing impregnated polyurethane that is not in the form of a foam in accordance with the invention.

In one aspect, and in some embodiments, the invention provides PU that is not in the form of a foam impregnated with a compound of Formula (I), wherein said PU is non-uniformly or non-homogenously impregnated with said compound of Formula (I). Alternatively viewed, in some aspects and embodiments, the invention provides PU that is not in the form of a foam impregnated with a compound of Formula (I), wherein said compound is not dispersed homogenously throughout the PU (or PU substrate). In one aspect, the invention provides PU, that is not in the form of a foam, impregnated with a compound of Formula (I), wherein there is a concentration gradient of the compound in the PU (or PU substrate), with higher concentrations being found in the outer layer(s) of the PU as compared to inner (or deeper) layer(s) of the PU.

In one aspect, and in some embodiments, the invention provides PU, that is not in the form of a foam, impregnated with a compound of Formula (I), wherein said compound is impregnated in said PU in (or over) only a portion of the total depth (or only a portion of the total depth from the surface) of the PU (PU substrate). Preferred depths are described elsewhere herein.

In some embodiments, the invention provides an antimicrobial article (e.g. a medical device) comprising (or consisting of) PU that is not in the form of a foam impregnated with a compound of Formula (I) produced by a method of producing impregnated polyurethane that is not in the form of a foam in accordance with the invention.

In some embodiments, the invention provides an antimicrobial article (e.g. a medical device) comprising (or consisting of) PU that is not in the form of a foam impregnated with a compound of Formula (I), wherein said PU is non-uniformly or non-homogenously impregnated with said compound of Formula (I). In some embodiments, the invention provides an antimicrobial article (e.g. a medical device) comprising (or consisting of) PU that is not in the form of a foam impregnated with a compound of Formula (I), wherein said compound is not dispersed homogenously throughout the PU (or PU substrate). In some embodiments, the invention provides an antimicrobial article (e.g. a medical device) comprising (or consisting of) PU, that is not in the form of a foam, impregnated with a compound of Formula (I), wherein there is a concentration gradient of the compound in the PU, with higher concentrations being found in the outer layer(s) of the PU as compared to inner layer(s) of the PU. In some embodiments, the invention provides an antimicrobial article (e.g. a medical device) comprising (or consisting of) PU that is not in the form of a foam impregnated with a compound of Formula (I), wherein said compound is impregnated in said PU in (or over) only a portion of the total depth (or only a portion of the total depth from the surface) of the PU.

As discussed elsewhere herein, the present inventors have also provided coating compositions (or painting compositions), which are liquid compositions, comprising at least one (e.g. 1 or 2) organic solvent (e.g. at least one polar aprotic solvent), a thermoplastic polyurethane (TPU) and a compound of Formula (I), wherein the TPU and a compound of Formula (I) are dissolved in said at least one organic solvent. These compositions can be used to “coat” or “paint” any desired article or part thereof (e.g. a medical device), which article may or may not itself be made of polyurethane (i.e. the material of the article to which the coating composition is applied is not necessarily polyurethane, although it may be polyurethane). Once the solvent(s) has evaporated, the surface of the article is left with a coating (or layer or surface layer or paint layer) comprising (or consisting of) thermoplastic polyurethane impregnated with a compound of Formula (I).

Thus, in one aspect, the present invention provides a composition (which may be conveniently referred to as a painting or coating composition, or formulation, or solution) comprising at least one organic solvent, a thermoplastic polyurethane (TPU) and a compound of Formula (I), wherein the TPU and compound of Formula (I) are (each) dissolved in said at least one organic solvent (or in said composition).

Alternatively viewed, the present invention provides a liquid composition comprising at least one organic solvent, a thermoplastic polyurethane (TPU) and a compound of Formula (I).

In some embodiments, the organic solvent is a polar aprotic solvent. For example, the polar aprotic solvent may be tetrahydrofuran (THF), dichloromethane (DCM), acetone, ethyl acetate, dimethyl sulfoxide (DMSO), N-methyl pyrrolidone (NMP), dimethyl formamide or dimethyl acetamide). In some embodiments, tetrahydrofuran (THF), dichloromethane (DCM), acetone or ethyl acetate are preferred. THF and DCM are preferred. THF is particularly preferred. In some embodiments, the organic solvent is an alcohol (e.g. ethanol) and/or chloroform (e.g. a mixture of alcohol and chloroform may be used, e.g. a mixture of ethanol and chloroform). In some embodiments, the organic solvent is not dimethyl formamide.

In some embodiments, only one solvent is present in the composition. In some embodiments in which only one solvent is present in the composition, said solvent is THF. In other embodiments, more than one different solvent is present (e.g. two different solvents may be present).

In embodiments in which more than one different solvent is present in the composition (e.g. two different solvents) the different solvents must be miscible (compatible). Combinations of miscible solvents can be readily selected.

In embodiments in which more than one different solvent is present in the composition (e.g. two different solvents), the combination of solvents may preferably be a combination (or miscible combination) of the solvents (or types of solvents) described above.

In some embodiments, there is only one type of organic solvent in the coating composition (or formulation). In other embodiments, there may be more than one different solvent in the coating composition (e.g. two different solvents, e.g. two different organic solvents). In embodiments in which there is more than one (e.g. 2) different solvents (e.g. more than one (e.g. 2) different organic solvents) in the formulation (or composition), the different solvents (e.g. different organic solvents) must be miscible (i.e. miscible with each other). Thus, in some embodiments, in which there is more than one (e.g. 2) different solvents (e.g. more than one (e.g. 2) different organic solvents) in the composition, the TPU and a compound of Formula (I) are soluble in the mixture of said more than one solvents. Miscible solvents may also be referred to as being “compatible” with each other. As is evident from discussion elsewhere herein, in some cases more than one different solvent may be present as the TPU and a compound of Formula (I) may each be dissolved in different solvents prior to being mixed together to produce a coating composition of the invention. In other cases, the TPU and a compound of Formula (I) may each be dissolved in the same solvent prior to being mixed together to produce a coating composition of the invention.

TPUs for use in coating compositions must be soluble in at least one organic solvent. Preferred TPUs are described elsewhere herein, and such preferred TPUs may be applied, mutatis mutandis, to the aspects and embodiments of the invention relating to coating compositions. For example, the TPU may be an aliphatic TPU, an aromatic TPU, an aliphatic polyether-based TPU, an aromatic polyether-based TPU, or an aromatic polycaprolactone copolyester-based TPU. Preferably, the TPU is a polymeric polyol-based TPU (e.g. as described elsewhere herein). Preferably the TPU is a medical grade TPU.

The amount of a compound of Formula (I) present in a coating composition of the invention should be sufficient to provide an antimicrobially effective amount (typically an antibacterially effect amount) of the compound in the impregnated PU coating once the coating composition has been applied to an article (or surface) and the solvent has evaporated.

In some embodiments, the concentration or amount (e.g. w/w) of a compound of Formula (I) in a coating composition (or solution) of the invention is at least 0.01%, at least 0.05%, at least 0.1%, at least 1%, at least 5%, at least 10% or at least 20% relative to the concentration or amount of the TPU. In some embodiments, the concentration or amount (e.g. w/w) of a compound of Formula (I) in a coating composition of the invention is up to 1%, up to 5%, up to 10%, up to 20% or up to 50% relative to the concentration or amount of the TPU. In some embodiments, the concentration or amount (e.g. w/w) of a compound of Formula (I) in a coating composition of the invention is 0.01%-50%, 0.05%-50%, 0.1%-50%, 1%-50%, 2%-50%, 5%-50%, 10%-50%, 20%-50%, 0.01%-20%, 0.05%-20%, 0.1%-20%, 1%-20%, 2%-20%, 5%-20%, 0.01%-10%, 0.05%-10%, 0.1%-10%, 1%-10%, 2%-10%, 0.01%-5%, 0.05%-5%, 0.1%-5%, 1%-5%, 2%-5%, relative to the concentration or amount of the TPU. In one exemplary embodiment, the concentration or amount (e.g. w/w) of a compound of Formula (I) in a coating composition of the invention is 5% (or approximately 5%) relative to the concentration or amount of the TPU.

In some embodiments, the ratio (w/w) of a compound of Formula (I) to TPU in a coating composition (or solution) of the invention is at least 1:10,000, at least 1:2000, at least 1:1000, at least 1:100, at least 1:20, at least 1:10 or at least 1:5. In some embodiments, the ratio (w/w) of a compound of Formula (I) to TPU in a coating composition (or solution) of the invention is up to 1:100, up to 1:20; up to 1:10, up to 1:5 or up to 1:2. In some embodiments, the ratio (w/w) of a compound of Formula (I) to TPU in a coating composition (or solution) of the invention is between 1:10,000-1:2, 1:2,000-1:2, 1:1,000-1:2, 1:100-1:2, 1:50-1:2, 1:20-1:2, 1:10-1:2, 1:5-1:2, 1:10,000-1:5, 1:2,000-1:5, 1:1,000-1:5, 1:100-1:5, 1:50-1:5, 1:20-1:5, 1:10,000-1:10, 1:2,000-1:10, 1:1,000-1:10, 1:100-1:10, 1:50-1:10, 1:10,000-1:20, 1:2,000-1:20, 1:1,000-1:20, 1:100-1:20, or 1:50-1:20.

In some embodiments, the concentration (e.g. w/v) of a compound of Formula (I) in a coating composition (or solution) of the invention is at least 0.25 mg/ml, at least 0.5 mg/ml, at least 1 mg/ml, at least 1.5 mg/ml, at least 2 mg/ml, at least 3 mg/ml, at least 4 mg/ml, at least 5 mg/ml, at least 10 mg/ml or at least 20 mg/ml. In some embodiments, the concentration (e.g. w/v) of a compound of Formula (I) in a coating composition (or solution) of the invention is at least 1 mg/ml, at least 1.5 mg/ml or at least 2 mg/ml (e.g. about 1 mg/ml, about 1.5 mg/ml or about 2 mg/ml). In some embodiments, the concentration (e.g. w/v) of a compound of Formula (I) in a coating composition (or solution) of the invention is 0.25 mg/ml to 5 mg/ml, 0.25 mg/ml to 10 mg/ml or 0.25 mg/ml to 20 mg/ml. In some embodiments, the concentration (e.g. w/v) of a compound of Formula (I) in a coating composition (or solution) of the invention is 1 mg/ml to 5 mg/ml, 1 mg/ml to 10 mg/ml or 1 mg/ml to 20 mg/ml. In some embodiments, the concentration (e.g. w/v) of a compound of Formula (I) in a coating composition (or solution) of the invention is 1.5 mg/ml to 5 mg/ml, 1.5 mg/ml to 2 mg/ml or 1.5 mg/ml to 20 mg/ml. In some embodiments, the concentration (e.g. w/v) of a compound of Formula (I) in a coating composition (or solution) of the invention is 2 mg/ml to 5 mg/ml, 2 mg/ml to 10 mg/ml or 2 mg/ml to 20 mg/ml. In some embodiments, the concentration (e.g. w/v) of a compound of Formula (I) in a coating composition (or solution) of the invention is up to 5 mg/ml, up to 10 mg/ml or up to 20 mg/ml.

In some embodiments, compositions (painting or coating compositions) in accordance with the present invention may further comprise one or more release enhancers. Release enhancers are agents that may enhance (or improve or facilitate) the release of a compound of Formula (I) from the (resultant) polyurethane coating (or polyurethane paint layer) that is produced by painting a surface (or device) with a composition in accordance with the invention. Release enhancers include, but are not limited to, polyethylene glycol (e.g. PEG 400 or PEG 1000).

In another aspect, the invention provides a method of producing a composition (coating composition) of the present invention.

In one aspect, the present invention provides a method of producing a composition (coating composition or painting composition or solution), said method comprising:
(i) dissolving a thermoplastic polyurethane (TPU) in an organic solvent;
(ii) dissolving a compound of Formula (I) in a solvent;
(iii) mixing the TPU dissolved in the organic solvent obtained in (i) with the compound of Formula (I) dissolved in the solvent obtained in (ii),
thereby producing a composition in accordance with the present invention. Embodiments of other aspects of the invention described herein apply, mutatis mutandis, to this aspect of the invention.

Any organic solvent that is capable of dissolving a TPU may be used in step (i) and any solvent that is capable of dissolving a compound of Formula (I) may be used in step (ii). Solvents used in step (i) and step (ii) must be miscible (or compatible) with each other.

In some embodiments, the organic solvent in step (i) is a polar aprotic solvent, e.g. as described elsewhere herein in connection with other aspects of the invention. THF and DCM are preferred organic solvents. THF is particularly preferred. In some embodiments, the organic solvent in step (i) is an alcohol (e.g. ethanol) and/or chloroform (e.g. a mixture of alcohol and chloroform may be used, e.g. a mixture of ethanol and chloroform).

In preferred embodiments, the solvent in step (ii) is an organic solvent, preferably a polar aprotic solvent (e.g. THF), e.g. as described elsewhere herein in connection with other aspects of the invention. In some embodiments, the solvent in step (ii) is an alcohol (e.g. ethanol) and/or chloroform (e.g. a mixture of alcohol and chloroform may be used, e.g. a mixture of ethanol and chloroform). In some embodiments the solvent in step (ii) is chloroform.

In some embodiments, the solvent of step (i) (i.e. the solvent used to dissolve the TPU) is different from the solvent of step (ii) (i.e. the solvent used to dissolve a compound of Formula (I)).

In some embodiments, the organic solvent of step (i) (i.e. the solvent used to dissolve the TPU) is the same as the solvent of step (ii) (i.e. the solvent used to dissolve a compound of Formula (I)). In some such embodiments, the solvent used in step (i) and step (ii) is a polar aprotic solvent, preferably THF.

Dissolving step (i) may be performed for any appropriate length of time to dissolve the TPU. For example, dissolving step (i) may be performed for at least 1 h, at least 2 h, at least 5 h, at least 12 h, at least 24 h, at least 48 h, at least 72 h or at least 92 h. If a given TPU has not dissolved over a given length of time, it may nevertheless still dissolve if left for additional time. Dissolution of the TPU may be assessed (or as assessed) by any appropriate means, including for example by visual inspection. The dissolved TPU obtained in step (i) may be viscous or it may be free-flowing (or non-viscous).

Dissolving step (ii) may be performed for any appropriate length of time to dissolve the compound. For example, dissolving step (ii) may be performed for at least 1 h, at least 2 h, at least 5 h, at least 12 h or at least 24 h. If a compound of Formula (I) has not dissolved over a given length of time, it may nevertheless still dissolve if left for additional time. Dissolution of the compound may be assessed (or as assessed) by any appropriate means, including for example by visual inspection.

Mixing step (iii) may be performed by any appropriate means, e.g. by mechanical stirring or by shaking.

Dissolving steps (i) and (ii) and mixing step (iii) may be performed at any appropriate temperature, e.g. ambient temperature.

In another aspect, the present invention provides a method of producing a coating composition (or solution) of the present invention, said method comprising (i) providing a first solution comprising a thermoplastic polyurethane (TPU) in an organic solvent; (ii) providing a second solution comprising a compound of Formula (I), wherein said second solution is miscible with the first solution; and (iii) mixing said first and second solutions. Embodiments of other aspects of the invention described herein apply, mutatis mutandis, to this aspect of the invention.

In another aspect, the present invention provides a method of producing a composition (coating composition or painting composition or solution), said method comprising dissolving a thermoplastic polyurethane (TPU) and a compound of Formula (I) in an organic solvent, and optionally mixing the solution obtained. Embodiments of other aspects of the invention described herein apply, mutatis mutandis, to this aspect of the invention.

In another aspect, the invention provides a composition produced by a method of producing a composition of the invention.

In another aspect, the invention provides the use of a coating composition of the invention to coat an article (or at least part of an article) or surface, e.g. an article or surface susceptible to microbial (e.g. bacterial) contamination or colonisation (such as a medical device).

In another aspect, the present invention also provides a coating (or dry coating or coating material or paint layer) for an article (e.g. medical device) or surface that may be susceptible to microbial contamination, wherein said coating comprises TPU impregnated with a compound of Formula (I). Preferably, such a coating is produced or formed by applying a coating composition of the invention to an article or surface and evaporating off (or drying off) the solvent from the applied composition, thereby providing a coating (dry coating). The evaporating (or drying) may be done at ambient temperature (i.e. passive drying) or alternatively an active evaporating (or drying) step may be performed. The evaporating may be performed for (or over) any suitable period of time (e.g. about 2 h, 5 h, 12 h, 1 day, 2 days, 3 days, 4 days, 5 days, 6 days or 7 days), Embodiments of other aspects of the invention described herein apply, mutatis mutandis, to this aspect of the invention.

In some embodiments, at least 0.01%, at least 0.05%, at least 0.1%, at least 1%, at least 5%, at least 10% or at least 20% of the total mass of the coating (or paint layer) is provided by a compound of Formula (I). In some embodiments, up to 5%, up to 10%, up to 20% or up to 50% of the total mass of the coating (or paint layer) is provided by a compound of Formula (I). In some embodiments, 0.01%-50%, 0.05%-50%, 0.1%-50%, 1%-50%, 2%-50%, 5%-50%, 10%-50%, 20%-50%, 0.01%-20%, 0.05%-20%, 0.1%-20%, 1%-20%, 2%-20%, 5%-20%, 0.01%-10%, 0.05%-10%, 0.1%-10%, 1%-10%, 2%-10%, 5%-10%, 0.01%-5%, 0.05%-5%, 0.1%-5%, 1%-5%, 2%-5% of the total mass of the coating (or paint layer) is provided by a compound of Formula (I).

In some embodiments, the coating (or dry coating or paint layer) has a thickness of at least 1 μm, at least 5 μm, at least 10 μm, at least 20 μm, at least 50 μm, at least 100 μm or at least 500 μm. In some embodiments, the coating (or dry coating or paint layer) has a thickness of up to 5 μm, up to 10 μm, up to 20 μm, up to 50 μm, up to 100 μm, up to 500 μm or up to 1000 μm. In some embodiments, the coating (or dry coating or paint layer) has a thickness of about 1 μm to about 5 μm, about 1 μm to about 10 μm, about 1 μm to about 20 μm, about 1 μm to about 50 μm, about 1 μm to about 100 μm, about 1 μm to about 500 μm or about 1 μm to about 1000 μm. In some embodiments, the coating (or dry coating or paint layer) has a thickness of about 5 μm to about 10 μm, about 5 μm to about 20 μm, about 5 μm to about 50 μm, about 5 μm to about 100 μm, about 5 μm to about 500 μm or about 5 μm to about 1000 μm. In some embodiments, the coating (or dry coating or paint layer) has a thickness of about 10 μm to about 20 μm, about 10 μm to about 50 μm, about 10 μm to about 100 μm, about 10 μm to about 500 μm or about 10 μm to about 1000 μm. In some embodiments, the coating (or dry coating or paint layer) has a thickness of about 20 μm to about 50 μm, about 20 μm to about 100 μm, about 20 μm to about 500 μm or about 20 μm to about 1000 μm. The thickness may be an average (e.g. mean) thickness. In some embodiments, the coating may have a uniform or substantially uniform (or homogenous or substantially homogenous) thickness. In other embodiments, the coating may have a non-uniform (or non-homogenous) thickness.

In one aspect, the invention provides a method of producing a coating for an article or surface that may be susceptible to microbial contamination, said method comprising (i) applying a composition (or coating composition or painting composition) of the invention to said article or surface and (ii) evaporating off (or drying off) the solvent from said applied composition. Embodiments of other aspects of the invention described herein apply, mutatis mutandis, to this aspect of the invention.

In another aspect, the invention provides a method of producing an article (e.g. medical device), or a surface, having an antimicrobial coating comprising thermoplastic polyurethane impregnated with a compound of Formula (I), said method comprising (i) providing a coating composition of the invention; and (ii) applying said composition to the article or surface, or to at least a part of the article or surface, (e.g. by dipping the article into said composition, or painting (e.g. spray painting) said composition onto the article). Typically, of course, the solvent(s) in the coating composition is then evaporated (or dried) off. Embodiments of other aspects of the invention described herein apply, mutatis mutandis, to this aspect of the invention.

Medical devices (such as catheters or other medical devices described herein) are particularly preferred articles having an antimicrobial coating of thermoplastic polyurethane impregnated with a compound of Formula (I).

A further aspect of the present invention is an article (typically an article that may be susceptible to microbial contamination), preferably a medical device, coated (which includes partially coated), with a coating composition of the invention as defined herein or a coating (e.g. dry coating) of the invention as defined herein. A further aspect of the present invention is an article, preferably a medical device, that has been coated (which includes partially coated), with a coating composition of the invention as defined herein. Typically, of course, the solvent(s) in the coating composition is, or has been, evaporated (or dried) off. Embodiments of other aspects of the invention described herein apply, mutatis mutandis, to this aspect of the invention.

In a yet further aspect of the invention provides a coating composition of the invention as defined herein which has been applied to an article, preferably to a medical device.

In a further aspect, and in some embodiments, the present invention provides an antimicrobial article (e.g. a medical device) comprising polyurethane, wherein said polyurethane is impregnated with a compound of Formula (I), wherein the polyurethane impregnated with a compound of Formula (I) is in the form of a coating (or surface layer) on said article. Preferably said coating is (or has been) formed by the application to said article of a coating composition (or painting composition) of the present invention (typically of course with the solvent(s) in said composition having been evaporated off). Embodiments of other aspects of the invention described herein apply, mutatis mutandis, to this aspect of the invention.

In another aspect, and in some embodiments, the invention provides an antimicrobial article (e.g. a medical device) that is coated, or at least partially coated, with a layer (or coating) of thermoplastic polyurethane impregnated with a compound of Formula (I). Embodiments of other aspects of the invention described herein apply, mutatis mutandis, to this aspect of the invention.

In embodiments of the invention which relate to coating (or coated) articles (e.g. medical devices) or surfaces, the article or surface (i.e. the underlying article or surface) to which the coating composition is (or has been applied) may be made of any material (or any material capable of being coated). The material of the article or surface to which the coating composition is applied (or has been applied) is not necessarily polyurethane, although it may be polyurethane in some cases. For example, in some embodiments, the article or surface may be made of silicone, e.g. the article may be a silicone medical device such as a silicone catheter (e.g. urinary catheter).

In another aspect, the invention provides a PU coating impregnated with a compound of Formula (I), wherein said PU coating impregnated with said compound is produced by a method of producing a PU coating in accordance with the invention.

In some embodiments, the invention provides an antimicrobial article, or a surface, comprising a PU coating impregnated with a compound of Formula (I), wherein said PU coating impregnated with said compound is produced by a method of producing a PU coating in accordance with the invention.

In another aspect, the present invention provides a method of producing polyurethane impregnated with a compound of Formula (I), said method comprising (i) providing a solution (or “spinning solution”) comprising polyurethane and a compound of Formula (I) and (ii) subjecting said solution to electrospinning, thereby producing (or extruding) polyurethane fibres that are impregnated with a compound of Formula (I). Optionally, said fibres may be formed into a sheet or a mesh or a mat (or the like). Embodiments of other aspects of the invention described herein apply, mutatis mutandis, to this aspect of the invention.

In another aspect, the present invention provides a method of producing polyurethane impregnated with a compound of Formula (I), said method comprising (i) providing a reaction mixture comprising (a) an alcohol with two or more reactive hydroxyl groups (—OH) per alcohol molecule (e.g. a diol, triol or other polyol, for example a polymeric polyol as described elsewhere herein; diols (preferably polymeric diols) are preferred), (b) an isocyanate that has more than one reactive isocyanate group (—NCO) per isocyanate molecule (e.g. a di- or tri-isocyanate or a polyisocyanate; diisocyanates are preferred) and (c) a compound of Formula (I), and (ii) subjecting said reaction mixture to conditions under which said alcohol of (a) and said isocyanate of (b) react with each other to produce (or form) polyurethane, thereby producing polyurethane impregnated with a compound of Formula (I). In some such methods, the compound of Formula (I) may be in an encapsulated form, e.g. as described elsewhere herein.

In some embodiments, an antimicrobial article in accordance with the present invention consists of a sheet (or layer or patch or pad or the like) comprising polyurethane (e.g. PU foam) impregnated with a compound of Formula (I). In such cases the methods of producing polyurethane impregnated with a compound of Formula (I) may be alternatively considered methods of producing an antimicrobial article in accordance with the invention.

In some other embodiments, polyurethane (e.g. PU foam) impregnated with a compound of Formula (I) are present in multi-component (e.g. multi-layer or more complex) antimicrobial articles, e.g. they may represent one layer (or one part) of a more complex antimicrobial article. Thus, in another aspect, the invention provides a method of producing an antimicrobial article of the invention, said method comprising (i) providing polyurethane (e.g. PU foam) impregnated with a compound of Formula (I) (for example produced as described herein) and (ii) incorporating (or combining) said impregnated polyurethane into a multi-component (e.g. multi-layer) antimicrobial article (e.g. combining it with additional absorbent layers and/or an outer layer (or cover layer or secondary layer or backing layer such as an adhesive backing layer). Embodiments of other aspects of the invention described herein apply, mutatis mutandis, to this aspect of the invention.

A further aspect provides polyurethane (e.g. PU foam, or a PU that is not in the form of a foam (a solid PU or non-foamed PU), or a PU coating such as a TPU coating) impregnated with a compound of Formula (I), or an antimicrobial article comprising such impregnated polyurethane, produced by a method of the invention. Embodiments of other aspects of the invention described herein apply, mutatis mutandis, to this aspect of the invention.

In another aspect (or alternatively viewed), the invention provides a polyurethane substrate which comprises a compound of Formula (I) as defined elsewhere herein. Embodiments of other aspects of the invention described herein apply, mutatis mutandis, to this aspect of the invention. For example, said substrate may be included in (or provided as coating or layer on) antimicrobial articles such as dressings or other medical devices, as described elsewhere herein.

A further aspect of the present invention provides an antimicrobial article of the invention for use in therapy.

“Therapy” includes treatment and prophylaxis, i.e. it includes both treatment and preventative uses.

In some embodiments, the invention provides an antimicrobial article of the invention for use in the treatment or prevention of an infection of a subject.

The infection may be an infection at any “physiological” site or surface that is susceptible to infection (typically bacterial infection).

In some preferred embodiments, the infection is a wound infection (e.g. infection of a wound as described elsewhere herein). Typically, when the infection is a wound infection, the antimicrobial article is a dressing (e.g. wound dressing).

In some embodiments, the infection may be a medical-device associated infection (e.g. an “in-dwelling” medical device associated infection). Typically, when the infection is a medical-device associated infection, the antimicrobial article is a medical device. Thus, in some embodiments, the infection is an infection at a site at which a medical device is (or has been) implanted (“in-dwelling” devices). Such devices include, for example, intrauterine devices, prostheses (e.g. prosthetic joints) and catheters (e.g. central venous or urinary catheters). In some embodiments the infection is an infection at a catheterisation site.

Preferably, the infection is a bacterial infection, for example an infection by Gram-positive bacteria (e.g. bacteria of the genus Staphylococcus or Streptococcus). In some embodiments, the infection is a Staphylococcus aureus infection. In some embodiments, the infection is a Staphylococcus epidermidis infection. In some embodiments, the infection is an infection by Gram-negative bacteria (e.g. bacteria of the genus Escherichia). In some embodiments, the infection is a Escherichia coli infection.

A further aspect of the present invention provides polyurethane impregnated with a compound of Formula (I) for use in inhibiting bacterial growth, for example in a wound of a subject. Embodiments of other aspects of the invention described herein apply, mutatis mutandis, to this aspect of the invention.

A further aspect of the invention provides polyurethane impregnated with a compound of Formula (I) for use in therapy, preferably for use in the treatment or prevention of an infection of a subject. In some embodiments, said polyurethane impregnated with a compound of Formula (I) is administered to (or applied to) a subject in the form of an antimicrobial article (e.g. a wound dressing or other medical device). Embodiments of other aspects of the invention described herein apply, mutatis mutandis, to this aspect of the invention.

A further aspect of the invention provides a compound of Formula (I) for use in therapy, preferably for use in the treatment or prevention of an infection of a subject, wherein said compound is administered to (or applied to) a subject in the form of polyurethane impregnated with said compound, or in the form of an antimicrobial article (e.g. a wound dressing or other medical device) comprising such impregnated polyurethane. Embodiments of other aspects of the invention described herein apply, mutatis mutandis, to this aspect of the invention.

A further aspect of the invention provides a compound of Formula (I) for use in therapy, preferably for use in the treatment or prevention of an infection of a subject, wherein said compound is administered to (or applied to) a subject in the form of a polyurethane coating that is impregnated with said compound, or in the form of an antimicrobial article (e.g. a medical device) comprising such an impregnated polyurethane coating. Embodiments of other aspects of the invention described herein apply, mutatis mutandis, to this aspect of the invention.

Alternatively viewed, the present invention provides a method of treating or preventing an infection which method comprises applying (or administering) to a subject in need thereof (e.g. to a wound of a subject in need thereof) an antimicrobial article in accordance with the present invention. Embodiments of other aspects of the invention described herein apply, mutatis mutandis, to this aspect of the invention.

Alternatively viewed, the present invention provides a method of treating or preventing an infection which method comprises applying (or administering) to a subject in need thereof a therapeutically effective amount of polyurethane impregnated with a compound of Formula (I). In some embodiments, said polyurethane impregnated with a compound of Formula (I) are administered to (or applied to) a subject in the form of an antimicrobial article (e.g. a wound dressing or other medical device). Embodiments of other aspects of the invention described herein apply, mutatis mutandis, to this aspect of the invention.

Alternatively viewed, the present invention provides a method of treating or preventing an infection which method comprises applying (or administering) to a subject in need thereof a therapeutically effective amount of a compound of Formula (I), wherein said compound is administered to (or applied to) a subject in the form of polyurethane impregnated with said compound, or in the form of an antimicrobial article (e.g. a wound dressing or other medical device) comprising such impregnated polyurethane. Embodiments of other aspects of the invention described herein apply, mutatis mutandis, to this aspect of the invention.

Alternatively viewed, the present invention provides a method of treating or preventing an infection which method comprises applying (or administering) to a subject in need thereof a therapeutically effective amount of a compound of Formula (I), wherein said compound is administered to (or applied to) a subject in the form of a polyurethane coating that is impregnated with said compound, or in the form of an antimicrobial article (e.g. a medical device) comprising such an impregnated polyurethane coating. Embodiments of other aspects of the invention described herein apply, mutatis mutandis, to this aspect of the invention.

A therapeutically effective amount will be determined based on the clinical assessment and can be readily monitored.

Further alternatively viewed, the present invention provides the use of polyurethane impregnated with a compound of Formula (I) as defined herein in the manufacture of an antimicrobial article (or medicament) for use in therapy. Preferred therapy is the treatment or prevention of infection, as described elsewhere herein. Embodiments of other aspects of the invention described herein apply, mutatis mutandis, to this aspect of the invention.

The present invention also provides the use of polyurethane impregnated with a compound of Formula (I) to inhibit (or prevent) bacterial colonization of an article (e.g. a medical device) or surface, wherein said article or surface comprises said impregnated polyurethane. Embodiments of other aspects of the invention described herein apply, mutatis mutandis, to this aspect of the invention.

The present invention also provides polyurethane impregnated with a compound of Formula (I) for use in inhibiting (or preventing) bacterial colonization of a medical device that has been applied to (or implanted in) a subject, wherein said medical device comprises said impregnated polyurethane. Embodiments of other aspects of the invention described herein apply, mutatis mutandis, to this aspect of the invention.

The present invention also provides a compound of Formula (I) for use in inhibiting (or preventing) bacterial colonization of a medical device that has been applied to (or implanted in) a subject, wherein said medical device comprises polyurethane impregnated with said compound. Embodiments of other aspects of the invention described herein apply, mutatis mutandis, to this aspect of the invention.

The present invention also provides a method of inhibiting (or preventing) bacterial colonization of an article (e.g. a medical device) or surface, said method comprising providing said article or surface with polyurethane that is impregnated with a compound of Formula (I). Embodiments of other aspects of the invention described herein apply, mutatis mutandis, to this aspect of the invention.

The term “subject” or “patient” as used herein includes any mammal, for example humans and any livestock, domestic or laboratory animal. Specific examples include mice, rats, pigs, cats, dogs, sheep, rabbits, cows and monkey. Preferably, however, the subject or patient is a human subject. Thus, subjects or patients treated in accordance with the present invention will preferably be humans.

In some embodiments, subjects or patients are those having an infection (e.g. having a wound infection or medical device-associated infection), or those suspected of having an infection (e.g. suspected of having a wound infection or medical device-associated infection), or those at risk of having (or contracting) an infection (e.g. those at risk of having a wound infection or medical device-associated infection).

In the context of therapeutic uses and methods of the present invention, the antimicrobial article (e.g. a wound dressing or other medical device) is typically applied (or affixed or secured or adhered) to a part of the subject's body (e.g. a wound) that is in need of infection prevention or treatment. Any suitable means may be used to apply (or secure) the antimicrobial article to the body.

The invention also provides kits comprising one or more of the antimicrobial articles of the invention. Preferably said kits are for use in the therapeutic methods and uses described herein. Preferably said kits comprise instructions for use of the kit components. Preferably said kits are for treating or preventing infection, e.g. as described elsewhere herein, and optionally comprise instructions for use of the kit components to treat such infections.

As used throughout the entire application, the terms “a” and “an” are used in the sense that they mean “at least one”, “at least a first”, “one or more” or “a plurality” of the referenced components or steps, except in instances wherein an upper limit is thereafter specifically stated.

In addition, where the terms “comprise”, “comprises”, “has” or “having”, or other equivalent terms are used herein, then in some more specific embodiments these terms include the term “consists of” or “consists essentially of”, or other equivalent terms.

The invention will now be further described with reference to the following non-limiting Examples and Figures, in which:

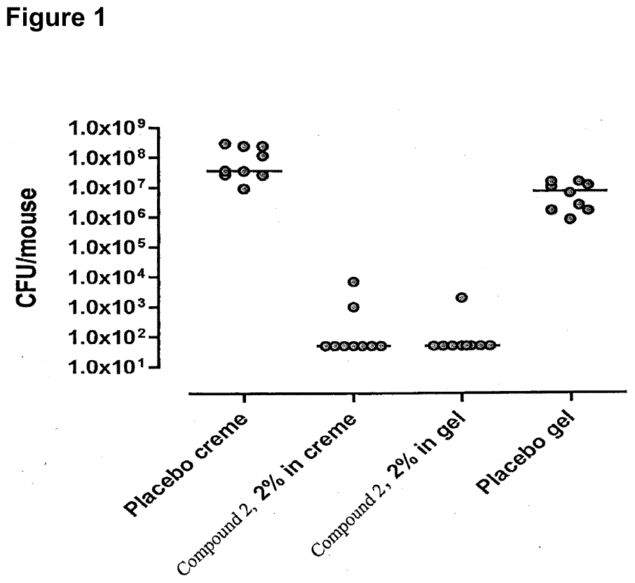

FIG. 1: is a graph showing the effect of one day topical treatment using compound 2 against Staphylococcus aureus FDA486 in a murine skin infection model. The number of colony forming units (CFU) are shown on the Y-axis and the type of topical treatment applied to the mice is shown on the X-axis. Compound 2 is also referred to herein as AMC-109.

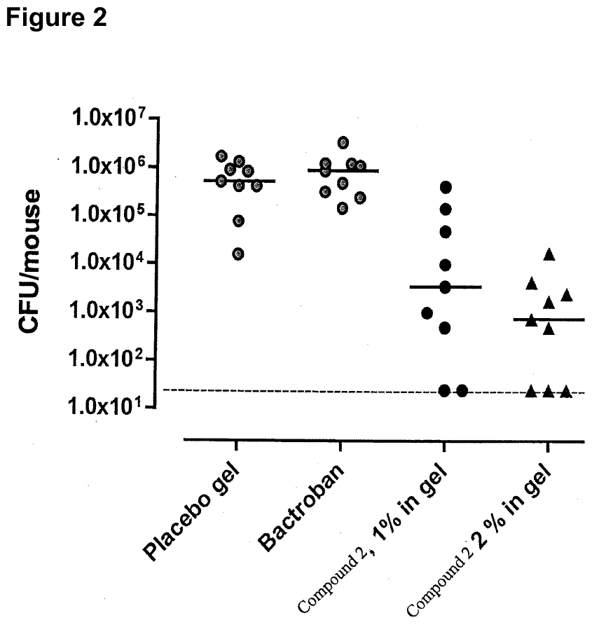

FIG. 2: is a graph showing the effect of one day topical treatment using compound 2 against Streptococcus pyogenes in a murine skin infection model. The number of colony forming units (CFU) are shown on the Y-axis and the type of topical treatment applied to the mice is shown on the X-axis. Compound 2 is also referred to herein as AMC-109.

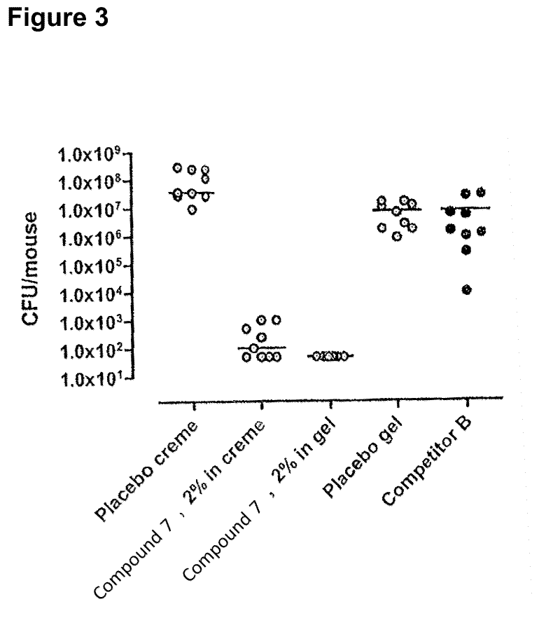

FIG. 3: is a graph showing the effect of one day topical treatment against S. aureus FDA486 in a murine skin infection model. Each mouse was treated at 9 am, 12 noon and 3 pm. The skin biopsy was collected at 6 pm. The median value is shown.

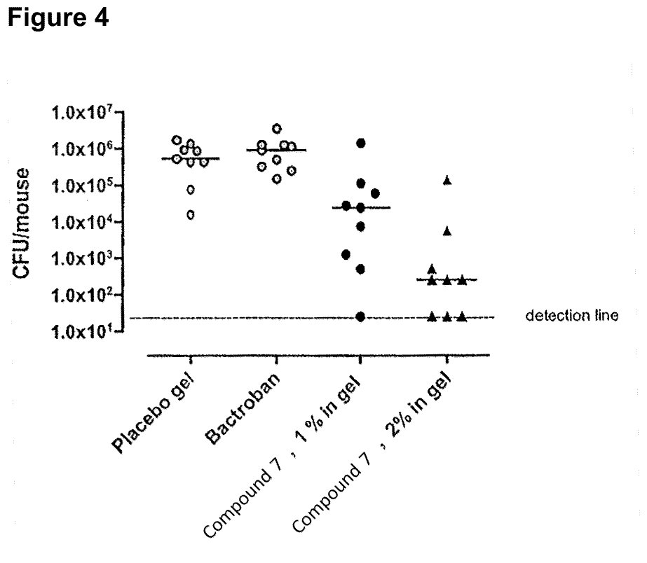

FIG. 4: is a graph showing the effect of one day topical treatment against Streptococcus pyogenes CS301 in a murine skin infection model. Each mouse was treated at 7 am, 10 am and 1 pm. The skin biopsy was collected at 4 pm. The median value is shown.

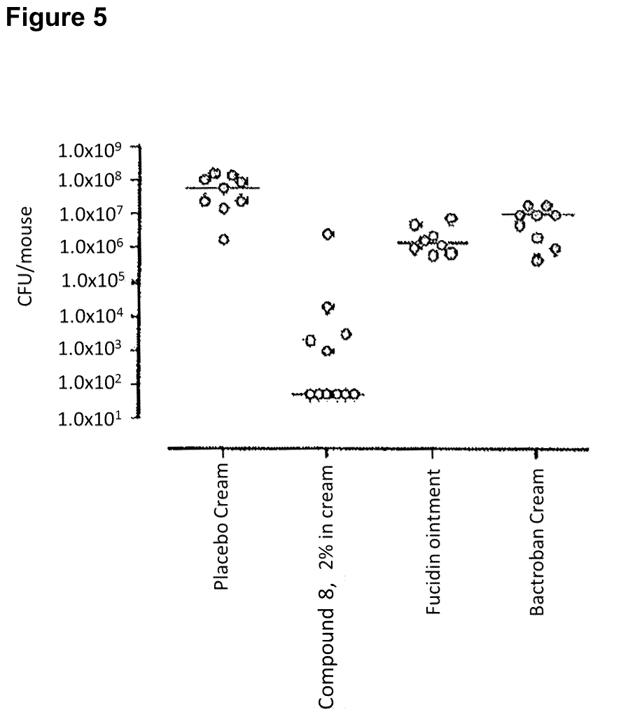

FIG. 5: is a graph showing the effect of one day topical treatment against S. aureus FDA486 in a murine skin infection model. Each mouse was treated at 9 am, 12 noon and 3 pm. The skin biopsy was collected at 6 pm. The median value is shown.

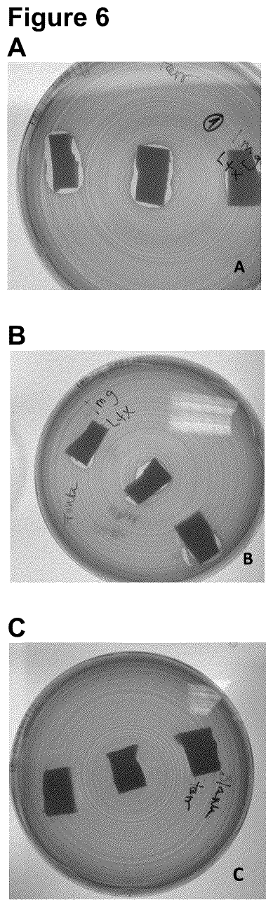

FIG. 6: Zone of inhibition around dry PU foam dressings impregnated with AMC-109 (A), moistened dressings impregnated with AMC-109 (B), and control dressings that are not impregnated with AMC-109 (C).

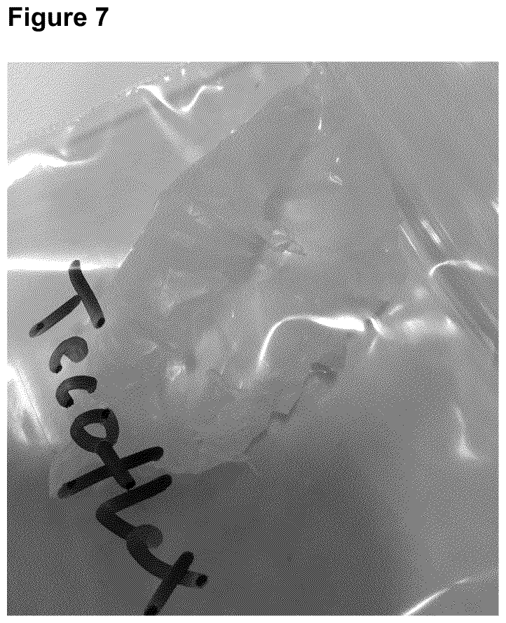

FIG. 7: shows an AMC-109 impregnated Tecoflex paint film (or coating), after drying, and after having being removed from its backing surface.

### EXAMPLES

### Example 1

### Peptide Synthesis

### Chemicals

Protected amino acids Boc-Trp-OH, Boc-Arg-OH, Boc-4-phenyl-Phe and Ac-Arg-OH were purchased from Bachem AG while Boc-4-iodophenylalanine, Boc-3,3-diphenylalanine and Boc-(9-anthryl)alanine were purchased from Aldrich. Benzylamine, 2-phenylethylamine, 3-phenylpropylamine, (R)-2-phenylpropylamine, (S)-2-phenylpropylamine, N,N-methylbenzylamine, N,N-ethylbenzylamine and N,N-dibenzylamine making up the C-terminal of the peptide were purchased from Fluka except N-ethylbenzylamine which was purchased from Acros. Diisopropylethylamine (DIPEA), 1-hydroxybenzotriazole (1-HOBt), chlorotripyrrolidinophosphonium hexafluorophosphate (PyCloP) and O-(benzotriazol-1-yl)-N,N,N′,N′ tetramethyluronium hexafluorophosphate (HBTU) were purchased from Fluka. 4-n-Butylphenylboronic acid, 4-t-butylphenylboronic acid, 4-biphenylboronic acid, 2-napthylboronic acid, tri ortho-tolylphosphine, benzylbromide and palladium acetate were purchased from Aldrich. Solvents were purchased from Merck, Riedel-de Haën or Aldrich.

### Preparation of Amino Acids

Preparation of Boc-2,5,7-tri-tert-butyltryptophan-OH: A mixture of H2N-Trp-OH (1.8 g, 8.8 mmol), t-BuOH (4.7 g, 63.4 mmol) in trifluoroacetic acid (19 mL) is stirred at 70° C. for 3 hours. The volume of the resulting mid-brown translucent solution is reduced on a rotary evaporator at room temperature for 30 min and then triturated by means of adding 60 mL of 7% (by weight) NaHCO3 drop-wise. The gray/white granular solid obtained is then recovered by vacuum filtration and dried in vacuo at room temperature for 24 hours. The product is isolated by crystallization from a near boiling mixture of 40% ethanol in water. Volumes typically are approximately 20 mL per gram of crude product.

A first crystallization from crude produces isolated product of 80-83% purity (HPLC) with respect to all other substances in the sample and approximately 94-95% purity with respect to the known TBT analogues. Yields at this stage are in the range 60-65%.

Benzylation of Boc-4-iodophenylalanine. Boc-4-iodophenylalanine (1 equivalent) was dissolved in 90% methanol in water and neutralized by addition of cesium carbonate until a weak alkaline pH (determined by litmus paper). The solvent was removed by rotary evaporation, and remaining water in the cesium salt of Boc-4-iodophenylalanine was further reduced by repeated azeotropic distillation with toluene. The resulting dry salt was dissolved in dimethylformamide (DMF), benzylbromide (1.2 equivalents) was added and the resulting mixture was stirred for 6-8 h. At the end of the reaction DMF was removed under reduced pressure and an oil containing the title compound is formed. This oil was dissolved in ethyl acetate and the resulting solution was washed with equal volumes of citric acid solution (three times), sodium bicarbonate solution and brine. The title compound was isolated as a pale yellow oil in 85% yield by flash chromatography using dichloromethane:ethyl acetate (95:5) as eluent. Crystalline benzyl Boc-4-iodophenylalanine could be obtained by recrystallisation from n-heptane.

General procedure for Suzuki couplings: Benzyl Boc-4-iodophenylalanine (1 equivalent), arylboronic acid (1.5 equivalents), sodium carbonate (2 equivalents), palladium acetate (0.05 equivalent) and tri ortho-tolylphosphine (0.1 equivalent) was added to a degassed mixture of dimethoxyethane (6 ml/mmol benzyl Boc-4-iodophenylalanine) and water (1 ml/mmol benzyl Boc-4-iodophenylalanine). The reaction mixture was kept under argon and heated to 80° C. for 4-6 h. After cooling to room temperature the mixture is filtered through a short pad of silicagel and sodium carbonate. The filter cake was further washed with ethyl acetate. The filtrates were combined and the solvents were removed under reduced pressure. The products were isolated by flash chromatography using mixtures of ethyl acetate and n-hexane as eluent.

Preparation of Boc-Bip(n-Bu)-OBn: The title compound was prepared in 53% yield from 4-n-butylphenylboronic acid using the general procedure for Suzuki couplings. Boc-Bip(n-Bu)-OBn was isolated using an 80:20 ethyl acetate:n-hexane eluent.

Preparation of Boc-Bip(t-Bu)-OBn: The title compound was prepared in 79% yield from 4-t-butylphenylboronic acid using the general procedure for Suzuki couplings. Boc-Bip(t-Bu)-OBn was isolated using an 80:20 ethyl acetate:n-hexane eluent.

Preparation of Boc-Bip(4-Ph)-OBn: The title compound was prepared in 61% yield from 4-biphenylboronic acid using the general procedure for Suzuki couplings. Boc-Bip(4-Ph)-OBn was isolated by recrystallisation of the crude product from n-heptane.

Preparation of Boc-Bip(4-(2-Naphtyl))-OBn: The title compound was prepared in 68% yield from 2-naphtylboronic acid using the general procedure for Suzuki couplings. Boc-Bip(4-(2-Naphtyl))-OBn was isolated by recrystallisation of the crude product from n-heptane.

Preparation of Boc-Bip(4-(1-Naphtyl))-OBn: The title compound was prepared from 2-naphtylboronic acid using the general procedure for Suzuki couplings. Boc-Bip(4-(1-Naphtyl))-OBn was isolated by recrystallisation of the crude product from n-heptane.

General procedure for deesterification of benzyl esters: The Benzyl ester is dissolved in DMF and hydrogenated for 2 days at ambient pressure using 10% Pd on carbon as catalyst. At the end of the reaction the catalyst is removed by filtration and the solvent is removed under reduced pressure. The free acids are isolated by recrystallisation from diethyl ether.

Preparation of Boc-Bip(4-n-Bu)-OH: The title compound was prepared in 61% yield from Boc-Bip(n-Bu)-OBn using the general procedure for deesterification.

Preparation of Boc-Bip(4-t-Bu)-OH: The title compound was prepared in 65% yield from Boc-Bip(t-Bu)-OBn using the general procedure for deesterification.

Preparation of Boc-Bip(4-Ph)-OH: The title compound was prepared in 61% yield from Boc-Bip(4-ph)-OBn using the general procedure for deesterification.

Preparation of Boc-Bip(4-(2-Naphtyl))-OH: The title compound was prepared in 68% yield from Boc-Bip(4-(2-Naphtyl))-OBn using the general procedure for deesterification.

Preparation of Boc-Bip(4-(2-Naphtyl))-OH: The title compound was prepared in 68% yield from Boc-Bip(4-(2-Naphtyl))-OBn using the general procedure for deesterification.

General procedure for Solution phase peptide synthesis using HBTU. The peptides were prepared in solution by stepwise amino acid coupling using Boc protecting strategy according to the following general procedure. The C-terminal peptide part with a free amino group (1 eq) and the Boc protected amino acid (1.05 eq) and 1-hydroxybenzotriazole (1-HOBt) (1.8 eq) were dissolved in DMF (2-4 ml/mmol amino component) before addition of diisopropylethylamine (DIPEA) (4.8 eq). The mixture was cooled on ice and O-(benzotriazol-1-yl)-N,N,N′,N′ tetramethyluronium hexafluorophosphate (HBTU) (1.2 eq) was added. The reaction mixture was shaken at ambient temperature for 1-2 h. The reaction mixture was diluted by ethyl acetate and washed with citric acid, sodium bicarbonate and brine. The solvent was removed under vacuum and the Boc protecting group of the resulting peptide was deprotected in the dark using 95% TFA or acetylchloride in anhydrous methanol.

Solution phase amide formation using PyCloP. Synthesis of Boc-Arg-N(CH₂Ph)₂. A solution of Boc-Arg-OH (1 eq), NH(CH₂Ph)₂ (1.1 eq) and PyCloP (1 eq) in dry DCM (filtered through alumina) (2 ml) and DMF (1 ml). The solution was cooled on ice and DIPEA (2 eq) was added under stirring. The solution was stirred for 1 h at room temperature. The reaction mixture was evaporated, and redissolved in ethyl acetate and washed with citric acid, sodium bicarbonate and brine. The solvent was removed under vacuum and the Boc protecting group of the resulting peptide was deprotected in the dark using 95% TFA.

Peptide purification and analysis. The peptides were purified using reversed phase HPLC on a Delta-Pak (Waters) C18 column (100 Å, 15 μm, 25×100 mm) with a mixture of water and acetonitrile (both containing 0.1% TFA) as eluent. The peptides were analyzed by RP-HPLC using an analytical Delta-Pak (Waters) C18 column (100 Å, 5 μm, 3.9×150 mm) and positive ion electrospray mass spectrometry on a VG Quattro quadrupole mass spectrometer (VG Instruments Inc., Altringham, UK).

### Example 2

### In Vitro Activities of Peptides Defined Herein

### Materials and Methods

### Antimicrobials

Vials of pre-weighed Compound 1 and Compound 2 were supplied by Lytix Biopharma AS.

General compound formula: AA₁-AA₂-AA₁-XY

| | AA₁ | AA₂ | XY |
|---|---|---|---|
| Compound 1 | Arg | Phe(4-(2-Naphtyl)) | NHCH₂CH₂Ph |
| Compound 2 | Arg | 2,5,7-tri-tert- | NHCH₂CH₂Ph |
| | | butyltryptophan | |

### Bacterial Isolates

Bacterial isolates used in this study were from various sources worldwide stored at GR Micro Ltd. and maintained, with minimal sub-culture, deep frozen at −70° C. as a dense suspension in a high protein matrix of undiluted horse serum. The species used and their characteristics are listed in Table 1. These included 54 Gram-positive bacteria, 33 Gram-negative bacteria and 10 fungi.

### Determination of Minimum Inhibitory Concentration (MIC)

MICs were determined using the following microbroth dilution methods for antimicrobial susceptibility testing published by the Clinical and Laboratory Standards Institute (CLSI, formerly NCCLS):

M7-A6 Vol. 23 No. 2 January 2003 Methods for Dilution Antimicrobial Susceptibility Tests for Bacteria that Grow Aerobically; Approved Standard—Sixth Edition. M100-S15 Vol. 25 No 1. January 2005 Performance Standards for Antimicrobial Susceptibility Testing; Fifteenth Informational Supplement. M11-A6 Vol. 24 No. 2 Methods for Antimicrobial Susceptibility Testing of Anaerobic Bacteria; Approved Standard—Sixth Edition. M27-A2 Vol. 22 No. 15 Reference Method for Broth Dilution Antifungal Susceptibility Testing of Yeasts; Approved Standard—Second Edition. M38-A Vol. 22 No. 16 Reference Method for Broth Dilution Antifungal Susceptibility Testing of Filamentous Fungi; Approved Standard.

MIC estimations were performed using wet plates, containing the antibacterials or antifungals, prepared at GR Micro Ltd.

Cation-adjusted Mueller-Hinton broth (Oxoid Ltd., Basingstoke, UK and Trek Diagnostic Systems Ltd., East Grinstead, UK) (supplemented with 5% laked horse blood for Streptococcus spp., Corynebacterium jeikeium and Listeria monocytogenes) was used for aerobic bacteria, with an initial inoculum of approximately 10⁵ colony-forming units (CFU)/mL.

Haemophilus test medium (Mueller-Hinton broth containing 0.5% yeast extract and Haemophilus test medium supplement which contains 15 mg/L of each of haematin and NAD, all obtained from Oxoid Ltd., Basingstoke, UK) was used for the Haemophilus influenzae and inoculated with approximately 10⁵ CFU/mL.

Supplemented Brucella broth (SBB) was used for the anaerobic strains with an inoculum of approximately 10⁶ CFU/mL. SBB is a broth consisting of 1% peptone, 0.5% ‘Lab-lemco’, 1% glucose and 0.5% sodium chloride supplemented with 5 μg/L haemin and 1 μg/L vitamin K (both obtained from Sigma Aldrich Ltd.) Yeast and filamentous fungal MIC were performed in MOPS buffered RPMI 1640 medium (MOPS buffer obtained from Sigma Aldrich Ltd., RPMI 1640 obtained from Invitrogen Ltd, Paisley, Scotland). The yeast inocula were in the range 7.5×10²-4×10³ CFU/mL and the filamentous fungi approximately 8×10³-1×10⁵ CFU/mL.

Following normal practice all the plates containing Mueller-Hinton broth were prepared in advance, frozen at −70° C. on the day of preparation and defrosted on the day of use. Fungal, Haemophilus and anaerobic MIC determinations were all performed in plates prepared on the same day.

To evaluate whether freezing affected the activity of the peptides some MIC determinations were repeated using plates containing freshly-prepared Mueller-Hinton broth.

### Control Strains

The following control (reference) strains were included in the panel of strains tested
Escherichia coli ATCC 25922
Staphylococcus aureus ATCC 29213
Enterococcus faecalis ATCC 29212
Streptococcus pneumoniae ATCC 49619
Pseudomonas aeruginosa ATCC 27853
Candida krusei ATCC 6258

The control strains below were extra to the test strain panel and were included where appropriate, to check that the comparators were within range.
Haemophilus influenzae ATCC 49247
Candida parapsilosis ATCC 22019
Bacteroides fragilis ATCC 25285
Eggerthella lenta ATCC 43055

### Results

The results are shown in Table 1 as a single line listing. Repeat control strain results are shown in Table 2. It can be seen that the control strain results were highly reproducible including data from plates that contained Mueller Hinton broth either stored frozen or used fresh. Freezing plates also had no effect on the MIC for other bacterial strains.

The MIC data obtained is very encouraging and indicates that the peptides have quite a broad spectrum of activity.

TABLE 1
Single line list of the in vitro activity of two antimicrobial
peptides and a comparator against a panel of Gram-positive
bacteria, Gram-negative bacteria and fungi.

| | Compound | Compound |
|---|---|---|
| Species and properties | 1 | 2 |
| Candida albicans ATCC90028 - | 2 | 8 |
| reference strain | | |
| Candida albicans ATCC24433 - | 2 | 8 |
| reference strain | | |
| Candida tropicalis ATCC750 - | 2 | 4 |
| reference strain | | |
| Candida parapsilosis ATCC90018 - | 4 | 16 |
| reference strain | | |
| Candida (Issatchenkia) krusei | 4 | 4 |
| ATCC6258 - reference strain | | |
| Aspergillus niger - G.R. Micro | 4 | 8 |
| collection | | |
| Trichophyton mentagrophytes - | 16 | 8 |
| G.R. Micro collection | | |
| Trichophyton interdigitale - | 8 | 8 |
| G.R. Micro collection | | |
| Microsporum canis - G.R. Micro | 8 | 8 |
| collection | | |
| Cryptococcus neoformans - G.R. | 4 | 4 |
| Micro collection | | |
| Escherichia coli ATCC25922 - | 32 | 8 |
| antibiotic-susceptible type strain | | |
| Escherichia coli ATCC32518 - | 32 | 8 |
| β-lactamase positive type strain | | |
| Escherichia coli - multi-drug | 32 | 8 |
| resistant clinical isolate | | |
| Klebsiella aerogenes NCTC11228 - | 32 | 16 |
| antibiotic-susceptible type strain | | |
| Klebsiella aerogenes - multi-drug | 64 | 16 |
| resistant clinical isolate | | |
| Enterobacter sp - antibiotic- | 32 | 4 |
| susceptible clinical isolate | | |
| Enterobacter sp - multi-drug | 64 | 16 |
| resistant clinical isolate | | |
| Pseudomonas aeruginosa ATCC27853 - | 16 | 8 |
| antibiotic-susceptible type | | |
| Pseudomonas aeruginosa - multi- | 32 | 4 |
| drug resistant clinical isolate | | |
| Stenotrophomonas maltophilia - | 64 | 8 |
| antibiotic-susceptible clinical isolate | | |
| Salmonella sp - antibiotic-susceptible | 16 | 8 |
| clinical isolate | | |
| Salmonella sp - multi-drug resistant | 16 | 8 |
| clinical isolate | | |
| Shigella sp - antibiotic-susceptible | 32 | 8 |
| clinical isolate | | |
| Morganella morganii - multi- | ≥128 | 16 |
| drug resistant clinical isolate | | |
| Haemophilus influenzae - β- | ≥128 | 8 |
| lactamase negative clinical isolate | | |
| Haemophilus influenzae - β - | ≥128 | 8 |
| lactamase positive clinical isolate | | |
| Haemophilus influenzae β- lactamase | ≥128 | 8 |
| negative ampicillin-resistant | | |
| Moraxella catarrhalis - β -lactamase | 4 | 4 |
| positive clinical isolate | | |
| Moraxella catarrhalis - reduced | 8 | 8 |
| fluoroquinolone susceptibility clinical | | |
| Acinetobacter baumanii - antibiotic- | 64 | 16 |
| susceptible clinical isolate | | |
| Staphylococcus aureus ATCC 29213 - | 4 | 2 |
| antibiotic-susceptible control | | |
| Staphylococcus aureus ATCC 25923 - | 4 | 4 |
| antibiotic-susceptible control | | |
| Staphylococcus aureus ATCC 43300 - | 4 | 2 |
| methicillin-resistant control strain | | |
| Staphylococcus aureus - methicillin- | 4 | 4 |
| resistant clinical isolate | | |
| Staphylococcus aureus - multi-drug- | 8 | 4 |
| resistant clinical isolate | | |
| Staphylococcus aureus - teicoplanin- | 4 | 4 |
| intermediate clinical isolate | | |
| Staphylococcus epidermidis antibiotic | 16 | 8 |
| susceptible clinical isolate | | |
| Staphylococcus epidermidis methicillin- | 2 | 4 |
| resistant clinical isolate | | |
| Staphylococcus haemolyticus - antibiotic | 4 | 4 |
| susceptible clinical isolate | | |
| Staphylococcus saprophyticus - antibiotic | 1 | 1 |
| susceptible clinical isolate | | |
| Enterococcus faecalis - ATCC 29212 | 4 | 4 |
| antibiotic-susceptible control | | |
| Enterococcus faecalis vancomycin- | 8 | 8 |
| susceptible clinical isolate | | |
| Enterococcus faecalis vancomycin- | 16 | 8 |
| resistant (VanA) clinical isolate | | |
| Enterococcus faecalis vancomycin- | 16 | 16 |
| resistant (VanB) clinical isolate | | |
| Enterococcus faecalis high-level | 16 | 8 |
| gentamicin-resistant clinical isolate | | |
| Enterococcus faecium vancomycin- | 8 | 8 |
| susceptible clinical isolate | | |
| Enterococcus faecium vancomycin- | 16 | 8 |
| resistant (VanA) clinical isolate | | |
| Enterococcus faecium vancomycin- | 8 | 4 |
| resistant (VanB) clinical isolate | | |
| Enterococcus gallinarum vancomycin- | 4 | 4 |
| resistant (VanC) clinical isolate | | |
| Streptococcus pneumoniae - ATCC 49619 | 32 | 16 |
| antibiotic-susceptible control | | |
| Streptococcus pneumoniae - penicillin- | 64 | 32 |
| susceptible clinical isolate | | |
| Streptococcus pneumoniae - penicillin- | 32 | 32 |
| intermediate clinical isolate | | |
| Streptococcus pneumoniae - penicillin- | 32 | 16 |
| resistant clinical isolate | | |
| Streptococcus pneumoniae - multi-drug | 64 | 32 |
| resistant clinical isolate | | |
| Streptococcus pyogenes - Macrolide | 32 | 16 |
| (MLS) resistant clinical isolate | | |
| Streptococcus pyogenes - Macrolide | 32 | 16 |
| (M-type) resistance clinical isolate | | |
| Corynebacterium jeikeium - antibiotic- | 32 | 16 |
| susceptible clinical isolate | | |
| Corynebacterium jeikeium - multi- | 32 | 8 |
| drug resistant clinical isolate | | |
| Listeria monocytogenes - antibiotic- | 32 | 16 |
| susceptible clinical isolate | | |
| MU50 Staphylococcus aureus (MRSA) - | 4 | 4 |
| VISA type strain | | |
| EMRSA3 Staphylococcus aureus (MRSA) - | 4 | 4 |
| SSCmec type 1 | | |
| EMRSA16 Staphylococcus aureus (MRSA) - | 4 | 4 |
| SSCmec type 2 | | |
| EMRSA1 Staphylococcus aureus (MRSA) - | 8 | 8 |
| SSCmec type 3 | | |
| EMRSA15 Staphylococcus aureus (MRSA) - | 4 | 4 |
| SSCmec type 4 | | |
| HT2001254 Staphylococcus aureus | 4 | 4 |
| (MRSA) - PVL positive | | |
| Streptococcus agalactiae - antibiotic- | 16 | 8 |
| susceptible clinical isolate | | |
| Streptococcus agalactiae - macrolide- | 32 | 16 |
| resistant clinical isolate | | |
| Group C Streptococcus - antibiotic- | 32 | 16 |
| susceptible clinical isolate | | |
| Group C Streptococcus - macrolide- | 64 | 32 |
| resistant clinical isolate | | |
| Group G Streptococcus - antibiotic- | 32 | 8 |
| susceptible clinical isolate | | |
| Group G Streptococcus - macrolide- | 32 | 16 |
| resistant clinical isolate | | |
| Streptococcus mitis - antibiotic- | 64 | 16 |
| susceptible clinical isolate | | |
| Streptococcus mitis - macrolide- | ≥128 | 32 |
| resistant clinical isolate | | |
| Streptococcus constellatus - | 64 | 32 |
| antibiotic-susceptible clinical isolate | | |
| Streptococcus constellatus - | 64 | 32 |
| macrolide-resistant clinical isolate | | |
| Streptococcus oralis - antibiotic- | 64 | 32 |
| susceptible clinical isolate | | |
| Streptococcus oralis - macrolide- | 32 | 32 |
| resistant clinical isolate | | |
| Streptococcus bovis - antibiotic- | 64 | 32 |
| susceptible clinical isolate | | |
| Streptococcus bovis - macrolide- | 8 | 8 |
| resistant clinical isolate | | |
| Streptococcus sanguis - antibiotic- | 64 | 32 |
| susceptible clinical isolate | | |
| Streptococcus sanguis - macrolide- | 32 | 32 |
| resistant clinical isolate | | |
| Clostridium perfringens - antibiotic- | ≥128 | 32 |
| susceptible clinical isolate | | |
| Clostridium difficile - antibiotic- | 32 | 16 |
| susceptible clinical isolate | | |

TABLE 2
In vitro activity of two antimicrobial peptides and
comparators against ATCC control strains (Including
ATCC control strains extra to the test strain panel)

| Strain | | Compound | | Plate |
|---|---|---|---|---|
| No. | Species and properties | 1 | 2 | type |
| GP01 | Staphylococcus aureus ATCC 29213 | 8 | 4 | Frozen |
| | antibiotic-susceptible control strain | | | MHB |
| GP01 | Staphylococcus aureus ATCC 29213 | 4 | 4 | Frozen |
| | antibiotic-susceptible control strain | | | MHB |
| GP01 | Staphylococcus aureus ATCC 29213 | 4 | 2 | Fresh |
| | antibiotic-susceptible control strain | | | MHB |
| GN01 | Escherichia coli ATCC 25922 | 32 | 8 | Frozen |
| | antibiotic-susceptible type strain | | | MHB |
| GN01 | Escherichia coli ATCC 25922 | 32 | 8 | Frozen |
| | antibiotic-susceptible type strain | | | MHB |
| GN01 | Escherichia coli ATCC 25922 | 16 | 8 | Fresh |
| | antibiotic-susceptible type strain | | | MHB |
| GN10 | Pseudomonas aeruginosa ATCC 27853 | 16 | 8 | Frozen |
| | antibiotic-susceptible type strain | | | MHB |
| GN10 | Pseudomonas aeruginosa ATCC 27853 | 32 | 8 | Frozen |
| | antibiotic-susceptible type strain | | | MHB |
| GN10 | Pseudomonas aeruginosa ATCC 27853 | 8 | 8 | Fresh |
| | antibiotic-susceptible type strain | | | MHB |
| GP11 | Enterococcus faecalis - ATCC 29212 | 8 | 8 | Frozen |
| | antibiotic-susceptible control strain | | | MHB |
| GP11 | Enterococcus faecalis - ATCC 29212 | 8 | 8 | Frozen |
| | antibiotic-susceptible control strain | | | MHB |
| GP11 | Enterococcus faecalis - ATCC 29212 | 4 | 4 | Fresh |
| | antibiotic-susceptible control strain | | | MHB |
| | Haemophilus influenzae - ATCC 47247 | 32 | 4 | HTM |
| | Candida parapsilosis ATCC 22019 | 4 | 8 | RPMI |
| | | | | 1640 |
| F05 | Candida (Issatchenkia) krusei ATCC | 8 | 8 | RPMI |
| | 6258 reference strain | | | 1640 |
| F05 | Candida (Issatchenkia) krusei ATCC | 8 | 8 | RPMI |
| | 6258 reference strain | | | 1640 |
| | Bacteroides fragilis - ATCC 25285 | 64 | 64 | SBB |
| | Eggerthella lenta - ATCC 43055 | 16 | 32 | SBB |

MHB, Mueller Hinton broth; HTM, haemophilus test medium; SBB, supplemented Brucella broth.

### Example 3

### Stability Towards Tryptic Degradation and Antimicrobial Activity

Compounds of formula AA₁-AA₂-AA₁-NHCH₂CH₂Ph were tested for their trypsin resistance and antimicrobial activity.

### Measurements and Calculation of Peptide Half-Life

Each peptide was dissolved in a 0.1 M NH₄HCO₃ buffer (pH 6.5) to yield a final peptide concentration of 1 mg/ml. A trypsin solution was prepared by dissolving 1 mg of trypsin in 50 ml 0.1 M NH₄HCO₃ buffer (pH 8.2). For the stability determination, 250 μl freshly made trypsin solution and 250 μl peptide solution were incubated in 2 ml of 0.1 M NH₄HCO₃ buffer (pH 8.6) at 37° C. on a rocking table. Aliquots of 0.5 ml were sampled at different time intervals, diluted with 0.5 ml water:acetonitrile (60:40 v/v) containing 1% TFA and analysed by RP-HPLC as described above. Samples without trypsin addition taken at 0 h and after 20 h at 37° C. were used as negative controls. Integration of the peak area at 254 nm for samples taken during the first 5 hours of the assay was used to generate the τ_{1/2}. Peptides that displayed no degradation during the first 24 h were classified as stable.

### Antibacterial Assay

MIC determinations on Staphylococcus aureus, strain ATCC 25923, Methicillin resistant Staphylococcus aureus (MRSA) strain ATCC 33591 and Methicillin resistant Staphylococcus epidermidis (MRSE) strain ATCC 27626 were performed by Toslab AS using standard methods. Amsterdam, D. (1996) Susceptibility testing of antimicrobials in liquid media, in Antibiotics in Laboratory Medicine. 4th ed (Lorian, V., Ed.) pp 75-78, Williams and Wilkins Co, Baltimore.

TABLE 3
Stability of AA₁-AA₂-AA₁-NHCH₂CH₂Ph peptides
towards trypsin measured as half-life (τ_{1/2})
and antibacterial activities displayed as MIC.

| | | | | MIC^{b} (μM) | | |
|---|---|---|---|---|---|---|
| Peptide | AA₁ | AA₂ | τ_{1/2}^{a} (h) | S. aureus^{c} | MRSA^{d} | MRSE^{e} |
| Com- | Arg | Trp | 7 | 145 | 97 | 81 |
| pound | | | | | | |
| 6^{f} | | | | | | |
| Com- | Arg | Bip(4-Ph) | Stable | 5 | 3 | 3 |
| pound | | | | | | |
| 5 | | | | | | |
| Com- | Lys | 2,5,7-tri-tert- | Stable | | 3 | <2 |
| pound | | butyltryptophan | | | | |
| 4 | | | | | | |
| Com- | Arg | Phe(4-(1- | 20 | | 3 | 3 |
| pound | | Naphtyl)) | | | | |
| 3 | | | | | | |
| Com- | Arg | 2,5,7-tri-tert- | Stable | <3 | <3 | <3 |
| pound | | butyltryptophan | | | | |
| 2 | | | | | | |
| Com- | Arg | Phe(4-(2- | Stable | 4 | <3 | <3 |
| pound | | Naphtyl)) | | | | |
| 1 | | | | | | |

^{a}Medical Calculator from Cornell University was used to calculate the half-life.
^{b}Minimal inhibitory concentration
^{c}Staphylococcus aures strain ATCC 25923
^{d}Methicillin resistant Staphylococcus aureus ATCC 33591
^{e}Methicillin resistant Staphylococcus epidermis ATCC 27626
^{f}not within compound definition for invention

### Example 4

### In Vivo Activity of Compound 2

The skin of mice was infected with Staphylococcus aureus or Streptococcus pyogenes and subsequently given a total of three treatments at three hourly intervals. Three hours after the last treatment, skin biopsies were collected and the number of colony forming units (CFUs) present in the skin sample was determined.

Results are shown in FIGS. 1 and 2, expressed as the number of colony forming units per mouse.

In experiment 1 (FIG. 1), compound 2 was applied to the murine skin as part of either a cream or a gel containing 2% (w/w) of compound 2. The same cream or gel without compound 2 was used as a negative control (placebo). It can clearly be seen that the number of CFUs was reduced when a cream or gel containing compound 2 was applied to the murine skin, compared to the negative control, indicating that compound 2 exerted an antimicrobial effect against Staphylococcus aureus. The nature of the carrier, cream or gel, had no significant effect.

In experiment 2 (FIG. 2), compound 2 was applied in two different concentrations, as either a 1% or a 2% gel. A placebo gel and a known antibacterial “bactroban” were used as controls. It can be seen that gels containing compound 2 were more effective at reducing the number of CFUs than the placebo gel or the bactroban. The gel containing 2% of compound 2 was more effective than the gel containing only 1% of compound 2.

### Example 5

### Preparation and Physical, Antimicrobial and Haemolytic Properties of Compounds of Use in the Invention

Peptide Synthesis—relevant information is also provided in Example 1.

### Chemicals

Protected amino acids Boc-Arg-OH, and Boc-4-phenyl-Phe were purchased from Bachem AG while Boc-4-iodophenylalanine was purchased from Aldrich. Isopropylamine, propylamine, hexylamine, butylamine, hexadecylamine, isobutylamine, cyclohexylamine and cyclopentylamine making up the C-terminal of the peptide were purchased from Fluka. Diisopropylethylamine (DIPEA), 1-hydroxybenzotriazole (1-HOBt), chlorotripyrrolidinophosphonium hexafluorophosphate (PyCloP) and O-(benzotriazol-1-yl)-N,N,N′,N′ tetramethyluronium hexafluorophosphate (HBTU) were purchased from Fluka. 4-n-Butylphenylboronic acid, 4-t-butylphenylboronic acid, 4-biphenylboronic acid, 2-napthylboronic acid, tri ortho-tolylphosphine, benzylbromide and palladium acetate were purchased from Aldrich. Solvents were purchased from Merck, Riedel-de Haën or Aldrich.

Preparation of Boc-Phe(4-4′-biphenyl)-OBn: The title compound was prepared in 61% yield from 4-biphenylboronic acid using the general procedure for Suzuki couplings. Boc-Phe(4-4′-biphenyl)-OBn was isolated by recrystallisation of the crude product from n-heptane.

Preparation of Boc-Phe(4-(2′-Naphtyl))-OBn: The title compound was prepared in 68% yield from 2-naphtylboronic acid using the general procedure for Suzuki couplings. Boc-Phe(4-(2′-Naphtyl))-OBn was isolated by recrystallisation of the crude product from n-heptane.

Preparation of Boc-Phe(4-4′-biphenyl)-OH: The title compound was prepared in 61% yield from Boc-Phe(4-4′-biphenyl)-OBn using the general procedure for deesterification.

Preparation of Boc-Phe(4-(2′-Naphtyl))-OH: The title compound was prepared in 68% yield from Boc-Phe(4-(2-Naphtyl))-OBn using the general procedure for deesterification.

General procedure for Solution phase peptide synthesis using HBTU is described in Example 1.

Solution phase amide formation using PyCloP is described in Example 1.

Peptide purification and analysis is described in Example 1.

TABLE 4
General compound formula: Arg-AA₂-Arg-X-Y

| Com- | | | Purity |
|---|---|---|---|
| pound | AA₂ | XY | (HPLC) |
| 7 | 2,5,7-tri-tert-butyltryptophan | NHCH(CH₃)₂ | |
| 8 | 2,5,7-tri-tert-butyltryptophan | NH(CH₂)₅CH₃ | |
| 9 | 2,5,7-tri-tert-butyltryptophan | NH(CH₂)₃CH₃ | 87 |
| 10 | 2,5,7-tri-tert-butyltryptophan | NH(CH₂)₂CH₃ | 99 |
| 11 | 2,5,7-tri-tert-butyltryptophan | NH(CH₂)₁₅CH₃ | 80 |
| 12 | 2,5,7-tri-tert-butyltryptophan | NHCH₂CH(CH₃)₂ | 97 |
| 13 | 2,5,7-tri-tert-butyltryptophan | NHcyclohexyl | 95 |
| 14 | 2,5,7-tri-tert-butyltryptophan | NHcyclopentyl | 91 |
| 15 | Phe(4-4′-biphenyl) | NHCH(CH₃)₂ | |
| 16 | Phe(4-4′-biphenyl) | NH(CH₂)₅CH₃ | |
| 17 | Phe(4-(2′-Naphtyl)) | NHCH(CH₃)₂ | |
| 18 | Phe(4-(2′-Naphtyl)) | NH(CH₂)₅CH₃ | |

### Antimicrobial Assay

MIC determinations on Staphylococcus aureus, strain ATCC 25923, Methicillin resistant Staphylococcus aureus (MRSA) strain ATCC 33591 and Methicillin resistant Staphylococcus epidermidis (MRSE) strain ATCC 27626 were performed by Toslab AS using standard methods. Amsterdam, D. (1996) Susceptibility testing of antimicrobials in liquid media, in Antibiotics in Laboratory Medicine. 4th ed (Lorian, V., Ed.) pp 75-78, Williams and Wilkins Co, Baltimore.

TABLE 5
Antimicrobial and toxic properties of compounds of use in the invention

| | C. albicans | S. aureus | MRSA | MRSE | S. pyogenes | E. coli | P. aeruginosa | |
|---|---|---|---|---|---|---|---|---|
| Compound | (mg/L) | (mg/L) | (mg/L) | (mg/L) | (mg/L) | (mg/L) | (mg/L) | EC50 |
| 7 | 25 | <2 | <2 | <2 | <2 | 7 | 7 | 720 |
| 8 | 5 | 2 | 2 | <1 | 2 | 5 | 5 | 32 |
| 9 | 10 | 2 | 3 | <2 | 2 | | | 350 |
| 10 | 10 | 2 | 3 | <2 | 2 | | | 620 |
| 11 | >100 | 5 | 4 | 4 | 6 | >100 | >100 | 38 |
| 12 | 10 | <2 | 3 | 2 | 2 | | | 300 |
| 13 | 10 | <2 | 2 | 2 | <2 | | | 55 |
| 14 | 10 | <2 | >15 | <2 | 2 | | | 340 |

### Example 6

### In Vitro Broad Panel Screening of Selected Compounds

### Materials and Methods

### Antimicrobials

Vials of pre-weighed Compound 7 and Compound 8 were supplied by Lytix Biopharma AS.

General compound formula: AA₁-AA₂-AA₁-X-Y

| | AA₁ | AA₂ | XY |
|---|---|---|---|
| Compound 7 | Arg | 2,5,7-tri-tert-butyltryptophan | NHCH(CH₃)₂ |
| Compound 8 | Arg | 2,5,7-tri-tert-butyltryptophan | NH(CH₂)₅CH₃ |

### Bacterial Isolates

Bacterial isolates used in this study are as described in Example 2.

### Determination of Minimum Inhibitory Concentration (MIC)

MICs were determined as described in Example 2.

### Results

The results are shown in Table 6 as a single line listing.

The MIC data obtained is very encouraging and indicates that the peptides have quite a broad spectrum of activity.

TABLE 6
Single line list of the in vitro activity of two
antimicrobial peptides against a panel of Gram-positive
bacteria, Gram-negative bacteria and fungi.

| | Compound | Compound |
|---|---|---|
| Species and properties | 7 (mg/L) | 8 (mg/L) |
| Candida albicans ATCC90028 - | 32 | 4 |
| reference strain | | |
| Candida albicans ATCC24433 - | 64 | 8 |
| reference strain | | |
| Candida tropicalis ATCC750 - | 4 | 4 |
| reference strain | | |
| Candida parapsilosis | 64 | 8 |
| ATCC90018 - reference strain | | |
| Candida (Issatchenkia) krusei | 8 | 32 |
| ATCC6258 - reference strain | | |
| Aspergillus niger - G.R. Micro | 32 | 4 |
| collection | | |
| Trichophyton mentagrophytes - | 8 | 4 |
| G.R. Micro collection | | |
| Trichophyton interdigitale - | 16 | 4 |
| G.R. Micro collection | | |
| Microsporum canis - G.R. Micro | 16 | 4 |
| collection | | |
| Cryptococcus neoformans - G.R. | 8 | 2 |
| Micro collection | | |
| Escherichia coli ATCC25922 - | 32 | 4 |
| antibiotic-susceptible type strain | | |
| Escherichia coli ATCC32518 - | 32 | 8 |
| β-lactamase positive type strain | | |
| Escherichia coli - multi-drug | 32 | 8 |
| resistant clinical isolate | | |
| Klebsiella aerogenes NCTC11228 - | 64 | 8 |
| antibiotic-susceptible type strain | | |
| Klebsiella aerogenes - multi-drug | 32 | 8 |
| resistant clinical isolate | | |
| Enterobacter sp - antibiotic- | 64 | 8 |
| susceptible clinical isolate | | |
| Enterobacter sp - multi-drug | ≥128 | 8 |
| resistant clinical isolate | | |
| Pseudomonas aeruginosa ATCC27853 - | 32 | 8 |
| antibiotic-susceptible type strain | | |
| Pseudomonas aeruginosa - multi-drug | 8 | 4 |
| resistant clinical isolate | | |
| Stenotrophomonas maltophilia - | 32 | 4 |
| antibiotic-susceptible clinical isolate | | |
| Salmonella sp - antibiotic-susceptible | 16 | 8 |
| clinical isolate | | |
| Salmonella sp - multi-drug resistant | 16 | 8 |
| clinical isolate | | |
| Shigella sp - antibiotic-susceptible | 32 | 4 |
| clinical isolate | | |
| Morganella morganii - multi-drug | 32 | 8 |
| resistant clinical isolate | | |
| Haemophilus influenzae - β- lactamase | 32 | 16 |
| negative clinical isolate | | |
| Haemophilus influenzae - β -lactamase | 16 | 4 |
| positive clinical isolate | | |
| Haemophilus influenzae β- lactamase | 16 | 8 |
| negative ampicillin-resistant clinical isolate | | |
| Moraxella catarrhalis - β -lactamase | 4 | 16 |
| positive clinical isolate | | |
| Moraxella catarrhalis - reduced | 8 | 16 |
| fluoroquinolone susceptibility clinical isolate | | |
| Acinetobacter baumanii - antibiotic- | 64 | 16 |
| susceptible clinical isolate | | |
| Staphylococcus aureus ATCC 29213 - | 8 | 4 |
| antibiotic-susceptible control strain | | |
| Staphylococcus aureus ATCC 25923 - | 8 | 4 |
| antibiotic-susceptible control strain | | |
| Staphylococcus aureus ATCC 43300 - | 8 | 4 |
| methicillin-resistant control strain | | |
| Staphylococcus aureus - methicillin- | 8 | 4 |
| resistant clinical isolate | | |
| Staphylococcus aureus - multi-drug- | 16 | 4 |
| resistant clinical isolate | | |
| Staphylococcus aureus - teicoplanin- | 16 | 4 |
| intermediate clinical isolate | | |
| Staphylococcus epidermidis antibiotic | 4 | 8 |
| susceptible clinical isolate | | |
| Staphylococcus epidermidis methicillin- | 4 | 2 |
| resistant clinical isolate | | |
| Staphylococcus haemolyticus - antibiotic | 4 | 4 |
| susceptible clinical isolate | | |
| Staphylococcus saprophyticus - antibiotic | 2 | 0.5 |
| susceptible clinical isolate | | |
| Enterococcus faecalis - ATCC 29212 | 16 | 4 |
| antibiotic-susceptible control strain | | |
| Enterococcus faecalis vancomycin- | 32 | 4 |
| susceptible clinical isolate | | |
| Enterococcus faecalis vancomycin- | 32 | 4 |
| resistant (VanA) clinical isolate | | |
| Enterococcus faecalis vancomycin- | ≥128 | 8 |
| resistant (VanB) clinical isolate | | |
| Enterococcus faecalis high-level | 64 | 8 |
| gentamicin-resistant clinical isolate | | |
| Enterococcus faecium vancomycin- | 16 | 4 |
| susceptible clinical isolate | | |
| Enterococcus faecium vancomycin- | 32 | 4 |
| resistant (VanA) clinical isolate | | |
| Enterococcus faecium vancomycin- | 16 | 4 |
| resistant (VanB) clinical isolate | | |
| Enterococcus gallinarum vancomycin- | 8 | 4 |
| resistant (VanC) clinical isolate | | |
| Streptococcus pneumoniae - ATCC 49619 | 32 | 16 |
| antibiotic-susceptible control strain | | |
| Streptococcus pneumoniae - penicillin- | 32 | 8 |
| susceptible clinical isolate | | |
| Streptococcus pneumoniae - penicillin- | 32 | 16 |
| intermediate clinical isolate | | |
| Streptococcus pneumoniae - penicillin- | 32 | 16 |
| resistant clinical isolate | | |
| Streptococcus pneumoniae - multi-drug | 32 | 16 |
| resistant clinical isolate | | |
| Streptococcus pyogenes - Macrolide | 16 | 8 |
| (MLS) resistant clinical isolate | | |
| Streptococcus pyogenes - Macrolide | 16 | 8 |
| (M-type) resistance clinical isolate | | |
| Corynebacterium jeikeium - antibiotic- | 8 | 4 |
| susceptible clinical isolate | | |
| Corynebacterium jeikeium - multi-drug | 8 | 2 |
| resistant clinical isolate | | |
| Listeria monocytogenes - antibiotic- | 16 | 8 |
| susceptible clinical isolate | | |
| MU50 Staphylococcus aureus (MRSA) - | 16 | 4 |
| VISA type strain | | |
| EMRSA3 Staphylococcus aureus (MRSA) - | 8 | 4 |
| SSCmec type 1 | | |
| EMRSA16 Staphylococcus aureus (MRSA) - | 16 | 4 |
| SSCmec type 2 | | |
| EMRSA1 Staphylococcus aureus (MRSA) - | 16 | 4 |
| SSCmec type 3 | | |
| EMRSA15 Staphylococcus aureus (MRSA) - | 8 | 4 |
| SSCmec type 4 | | |
| HT2001254 Staphylococcus aureus | 8 | 4 |
| (MRSA) - PVL positive | | |
| Streptococcus agalactiae - antibiotic- | 8 | 8 |
| susceptible clinical isolate | | |
| Streptococcus agalactiae - macrolide- | 16 | 8 |
| resistant clinical isolate | | |
| Group C Streptococcus - antibiotic- | 16 | 8 |
| susceptible clinical isolate | | |
| Group C Streptococcus - macrolide- | 32 | 16 |
| resistant clinical isolate | | |
| Group G Streptococcus - antibiotic- | 16 | 8 |
| susceptible clinical isolate | | |
| Group G Streptococcus - macrolide- | 16 | 8 |
| resistant clinical isolate | | |
| Streptococcus mitis - antibiotic- | 32 | 16 |
| susceptible clinical isolate | | |
| Streptococcus mitis - macrolide- | 64 | 16 |
| resistant clinical isolate | | |
| Streptococcus constellatus - | 64 | 16 |
| antibiotic-susceptible clinical isolate | | |
| Streptococcus constellatus - macrolide- | 32 | 16 |
| resistant clinical isolate | | |
| Streptococcus oralis - antibiotic- | 64 | 16 |
| susceptible clinical isolate | | |
| Streptococcus oralis - macrolide- | 64 | 16 |
| resistant clinical isolate | | |
| Streptococcus bovis - antibiotic- | 32 | 8 |
| susceptible clinical isolate | | |
| Streptococcus bovis - macrolide- | 8 | 2 |
| resistant clinical isolate | | |
| Streptococcus sanguis - antibiotic- | 32 | 16 |
| susceptible clinical isolate | | |
| Streptococcus sanguis - macrolide- | 32 | 16 |
| resistant clinical isolate | | |
| Clostridium perfringens - antibiotic- | ≥128 | 32 |
| susceptible clinical isolate | | |
| Clostridium difficile - antibiotic- | 64 | 32 |
| susceptible clinical isolate | | |
| Propionibacterium acnes- antibiotic- | | 4 |
| susceptible clinical isolate | | |
| Propionibacterium acnes- antibiotic- | | 2 |
| resistant clinical isolate | | |

### Example 7

### In Vivo Activity of Compounds 7 and 8

The skin of mice was infected with Staphylococcus aureus or Streptococcus pyogenes and subsequently given a total of three treatments at three hourly intervals. Three hours after the last treatment, skin biopsies were collected and the number of colony forming units (CFUs) present in the skin sample was determined. Results are shown in FIGS. 3, 4 and 5 expressed as the number of colony forming units per mouse.

In experiment 1 (FIG. 3), compound 7 was applied to the murine skin as part of either a cream or a gel containing 2% (w/w) of compound 7. The same cream or gel without compound 7 was used as a negative control (placebo). Bactroban 2% cream was used as a positive control. It can clearly be seen that the number of CFUs was reduced when a cream or gel containing compound 7 was applied to the murine skin, compared to the negative control, indicating that compound 7 exerted an antimicrobial effect against Staphylococcus aureus. The efficacy of standard clinical treatment, Bactroban 2% cream, had no significant effect under the treatment regime. The nature of the carrier, cream or gel, had no significant effect.

In experiment 2 (FIG. 4), compound 7 was applied in two different concentrations, as either a 1% or a 2% gel. A placebo gel and a known antibacterial “bactroban (mupericin)” were used as controls. It can be seen that gels containing compound 7 were more effective at reducing the number of CFUs from a Streptococcus pyogenes CS 301 infection than the placebo gel or the bactroban. The gel containing 2% of compound 7 was more effective than the gel containing only 1% of compound 7.

In experiment 3 (FIG. 5), compound 8 was applied in a 2% cream formulation on a Staphylococcus aureus FDA 486 infection in the murine skin infection model. A placebo cream and two known antibacterials, “Fucidin (fucidic acid) ointment 2%” and “Bactroban (mupericin) cream 2%” were used as controls. It can be seen that a cream containing compound 8 was more effective at reducing the number of CFUs than the placebo and fucidin or bactroban.

### Example 8

### Impregnated Polyurethane Foam

### Material and Methods:

Bacterial strain: S. aureus ATCC29213.

PU-foam wound dressing (Biatain, Coloplast) naive or impregnated with AMC-109.

### Preparation of PU-Foam Patches

Commercially available absorbing PU-foam wound dressing (Biatain, Coloplast) was cut into 1 cm square patches (1 cm²) using a sharp scalpel. The patches were impregnated with AMC-109 by careful addition of 0.5 ml of a 2 mg/ml solution of AMC-109 in distilled water onto each patch. The patches were then allowed to dry and regain their original shape before the microbiological tests.

### Microbiology

The Staphylococcus aureus was diluted to 0.5 McFarland and spread on Mueller Hinton agar plates to provide inoculated plates.

In order to investigate if the bactericidal effect of the AMC-109 impregnation was improved by moistening the dressing, 100 μl of NaCl was added to the dressing before applying the dressings to the inoculated plate.

Moistened and dry dressings impregnated with AMC-109, and naive controls (i.e. control dressings not impregnated with AMC-109), were placed on inoculated plates. All experiments were performed in triplicates. Plates were incubated at 37° C. for 16 hours. The MH-agar plates were then inspected for inhibition of bacterial growth and photographed for documentation.

### Results:

A clear zone of inhibition (4 mm), was observed around the dry PU-foam dressing, impregnated with 1 mg/cm² AMC-109 (FIG. 6A).
A clear zone of inhibition (2 mm), was observed around the moistened PU-foam dressing, impregnated with 1 mg/cm² AMC-109 (FIG. 6B).
No zone of inhibition was observed around the control dressings (FIG. 6C).

### Conclusion:

The PU-foam dressing impregnated with AMC-109 (1 mg/cm²) clearly inhibited bacterial growth underneath the dressing as well as creating a surrounding inhibition zone. This result demonstrates that AMC-109 is liberated from the foam to the underlying agar and eliminates the bacteria. This effect is not improved by pre-moistening the dressing suggesting that the moisture present in the incubator is sufficient for releasing AMC-109 from the dry dressing.

These findings support the use of AMC-109 impregnated PU-foam dressings as a method for controlling colonization of the dressing, and also for the prevention or treatment of infections in the underlying wound to which such AMC-109 dressings are applied.

### Example 9

### Example of Impregnated Polyurethane (Non-Foam)

### Introduction

As demonstrated in Example 8, the antimicrobial peptide AMC-109 can be impregnated in polyurethane foam and the peptide can be liberated from the polyurethane foam and exert antimicrobial activity.

A further polyurethane impregnation study has been performed, as described below.

To make the use of the peptide more economical (the peptide is relatively expensive), it could be desirable in some circumstances to impregnate just the superficial layers of the material (polyurethane), particularly in thick walled products.

In the present study it has been shown that the absorption of the AMC-109 containing swelling agent (AMC-109 containing solution) by polyurethane can be controlled by controlling the swelling time, and that even with short swelling times, AMC-109 impregnated polyurethane has good antimicrobial activity.

### Materials and Methods

### Manufacture of Impregnated Polyurethane

The polyurethane used was Pellethane 80 A sheeting (Lubrizol Advanced Materials, Inc., USA), with a thickness of 1 mm. Pellethane is a biocompatible aromatic polyether-urethane. The sheet was cut to samples of 2×1 cm. The Pellethane used in this study is not a foam.

A mix of 0.18 g AMC-109 in 4 ml ethanol and 2 ml chloroform resulted in a 2.8% solution.

The polyurethane samples were soaked in the swelling agent (i.e. the AMC-109 containing ethanol/chloroform which contains AMC-109) for two different times resulting in different absorptions (i.e. different degrees of swelling of the polyurethane) with the aim of achieving absorption percentages of 10% and 100%.

After soaking, the samples were superficially dried with cellulose wipes, weighed, dried at 20° C. for 12 hours, bagged and labeled.

“% absorption” is the % increase in weight of the polyurethane after the application of the solution of a compound of Formula (I) (i.e. after the swelling), i.e. as compared to the weight of the polyurethane prior to the application of the swelling agent (i.e. before the swelling).

### Microbiological Testing of Impregnated Polyurethane

Overnight colonies of S. epidermidis (Staphylococcus epidermidis RP62A) were diluted to 0.5 McFarland (1×10⁸ CFU) in 0.85% NaCl. The bacterial solution was further diluted in Tryptic Soy Broth media (1×10⁵) and drops of 100 μl were applied to the surfaces of the polyurethane samples. The polyurethane samples were placed on glass microscope slides, and the glass slides were placed in a moist incubation chamber and incubated for 24 hours at 37° C.

For determining CFU (colony forming units), the polyurethane samples (after incubation with the bacterial solution as described above) were vortexed in 2 ml 0.85% NaCl and serial dilutions (10⁻¹ to 10⁻⁶) were made. 100 μl aliquots of the serial dilutions were streaked on blood agar plates, and further incubated overnight, prior to CFU counting. All experiments were performed twice.

### Results and Discussion

Table A—Sample numbers, percentage of AMC-109 in swelling agent, exposure time (or soaking/swelling time), initial weight of sample (total of 4 pieces in 37/20, 3 pieces in 42/20), weight of sample after exposure, and absorption percentage.

| | Swelling | | | | Weight | |
|---|---|---|---|---|---|---|
| | agent | | | | after | |
| Sample | AMC-109 | Exposure | | Initial | swelling | % |
| No. | content | time | | weight (g) | (g) | absorption |
| 37/20 | 2.8% | 3 | min | 1.029 | 1.133 | 10.1% |
| 42/20 | 2.8% | 10 | h | 0.731 | 1.398 | 91.2% |

| | |
|---|---|
| 49/20 | Control. No swelling performed |

These results show that a swelling agent containing AMC-109 can swell polyurethane and that different exposure times (swelling times) can result in different degrees of absorption (swelling).

Both samples 37/20 (i.e. exposure/swelling time of 3 min) and 42/20 (i.e. exposure/swelling time of 10 h) showed efficient antimicrobial activity in the microbiological assay. The control sample showed a high level of bacterial surface colonisation.

Without wishing to be bound by theory, it is believed that during the swelling process a concentration gradient is present with the outer layer of the polyurethane (i.e. the polyurethane at, or closest to, to the surface) being saturated and the inner layers (furthest from the surface) being only partially saturated. By stopping the swelling process before total saturation of the whole polyurethane sample/article is reached, it is believed a polyurethane sample/article is obtained in which the surface/outer layers contains a desired concentration of peptide, which may be determined by the peptide concentration of the swelling agent and the total absorption at saturation. It is believed that the inner layers of the polyurethane (i.e. the polyurethane further/furthest from the surface) would contain less peptide than at the surface/outer layers. Put another way, and again without being bound by theory, it is believed that by controlling the swelling time the degree to which (or depth to which) the swelling agent (and thus the peptide) penetrates the polyurethane can be controlled. Thus, it is believed that by controlling the swelling time, impregnation of polyurethane can be controlled to achieve, if desired, impregnation of only the surface/outer layers of polyurethane. Given the cost of the materials (e.g. the cost of the peptide), impregnating only the surface and outer layers of polyurethane (i.e. impregnating to only a portion of full thickness of the polyurethane) would have a cost benefit as compared to impregnating the entire polyurethane product (i.e. the entire thickness of the polyurethane sample). The thickness of the impregnated portion (or layer) could then determine the leaching rate of the peptide and the time-to-depletion of the peptide. Impregnation of a polyurethane article with an antimicrobial peptide throughout the entire thickness of the article may not be necessary for all intended uses.

### Example 10

The purpose of this study is to investigate whether TPU (thermoplastic polyurethane) polymers can be dissolved in solvents compatible with AMC-109 dissolution. The results shows that THF (tetrahydrofuran) and dichloromethane (DCM) are quite general solvents for dissolution, and that painting solutions (i.e. dissolved AMC-109 and TPU in solvent) can be applied on surfaces to provide a painted surface (coating). This study also shows that AMC-109 can be extracted from (i.e. can leach out of) the paints (coatings) and that the AMC109/TPU paint (coating) has good anti-colonising activity.

### Materials and Methods

### Sample Materials

Tecoflex is a commercially medical grade available TPU (Lubrizol). The specific type of Tecoflex used was Tecoflex EG-80A. Pearlcoat DIPP 119 is a commercially available TPU (Lubrizol). Estane 58300 is a commercially available TPU (Lubrizol). Characteristics of the sample material are set forth in Table C.

TABLE C
Samples used for dissolution tests.

| Sample | Description |
|---|---|
| Estane | A complete polyester and polyether based TPU product line |
| 58300 | primarily designed for extrusion and injection molding. |
| Pearlcoat | An aromatic polycaprolactone copolyester-based |
| Dipp 119 | thermoplastic polyurethane. |
| Tecoflex | Aliphatic polyether-based thermoplastic polyurethane. |

### Dissolution Experiments

A sample of each TPU material (0.01-0.2 g) was placed in a vial containing either tetrahydrofurane (THF), dichloromethane (DCM), acetone, ethyl acetate (EtOAc) or ethanol (EtOH). The solubility was assessed visually after 48 h at ambient temperature.

### Preparation of Painting Solutions

The selected TPU sample material was dissolved in THF. In this regard, Tecoflex (1.0 g) was dissolved in THF (tetrahydrofuran) (40 ml), or Pearlcoat DIPP 119 (1.0 g) was dissolved in THF (25 ml) (Table D). AMC-109 (53 mg) was also dissolved in THF (4 ml) (Table D). The AMC-109 dissolved in THF was then mixed with the TPU (i.e. Tecoflex or Pearlcoat DIPP 119) dissolved in THF. This mixture is referred to as a “painting solution” (or “paint solution” or “final painting solution”). The concentration of AMC-109 in the final painting solution was 5% relative to the TPU. The dissolution of AMC-109 in THE takes several hours.

TABLE D
Solvents used for preparation of paint solutions.

| | Solvent for dissolving | Solvent for dissolving |
|---|---|---|
| Sample (type of TPU) | TPU | AMC-109 |
| Estane 58300 | THF | THF |
| Pearlcoat Dipp 119 | THF | THF |
| Tecoflex | THF | THF |

### Painting Procedure

The paint samples were prepared by placing 8 ml of the painting solution on aluminium foil with a shallow indentation or by pouring the painting solution on a watch glass. After drying (several days), the quite thick paint film could be mechanically loosened from its surface. FIG. 7 shows an AMC-109 impregnated Tecoflex paint film (or coating), after drying, and after having being mechanically removed from its backing surface.

### Extraction

Samples were cut from the paint film and accurately weighed (100-150 mg), and the amount of AMC-109 in the sample was calculated. The samples were placed in vials, water (2 ml) was added and the vials shaken. Six consecutive extractions were performed. For each extraction the old extract was replaced by deionized water (2 ml). The extractions were performed with a shaking period of 10 s, 5 min, 30 min, 3 h, 22 h, and 48 h. The amount of AMC-109 in each extract was determined by UV-spectrophotometry at 280 nm using a pre-made standard curve.

### Microbiology

### Bacterial Strain:

Staphylococcus aureus 8325

### Modified AATCC-100 Method

Overnight colonies of S. aureus were diluted to 0.5 McFarland in 0.9% NaCl resulting in a bacterial concentration of 1.5×10⁸ CFU/ml. This suspension was further diluted in TSB (tryptic soy broth) to 1×10⁵ CFU/ml.

The TPU material (i.e. the paint film containing AMC-109) was cut into pieces of approximately 0.4×0.4 cm. The material was then submerged in dH₂O for 2 minutes and air dried before use. The different samples (materials) were inoculated with 100 μl of the bacterial solution (i.e. 100 μl 1×10⁵ CFU/ml suspension). The samples were placed on a glass slide and incubated in a moisture chamber at 37° C. for 24 hours. Two biological replicates of each test material were made.

After incubation the TPU material was placed in 1000 μl NaCl (0.9%) and vortexed for 45 seconds, before making serial dilutions (0-10⁻⁶) and plating of 100 μl for CFU counting.

### Results

The dissolution of the TPU materials in various solvents after 48 hours at ambient temperature are compiled in Table E.

TABLE E
Dissolution of TPU materials in various solvents.

| Sample | THF | DCM | Acetone | EtOAc | EtOH |
|---|---|---|---|---|---|
| Estane | Dissolved | Swelled | Not | Not | Not |
| 58300 | Viscous | | dissolved | dissolved | dissolved |
| Pearlcoat | Dissolved | Dissolved/ | Dissolved | Dissolved/ | Not |
| Dipp 119 | Free | Swelled | Very | Swelled | dissolved |
| | flowing | | viscous | | |
| Tecoflex | Dissolved | Dissolved | Not | Not | Not |
| | Very | Very | dissolved | dissolved | dissolved |
| | viscous | viscous | | | |

Tetrahydrofuran (THF) appears to be the most effective solvent for the TPU samples. AMC-109 dissolves in THF, albeit very slowly. Dichloromethane (DCM) also dissolved TPU samples quite effectively. Experiments also showed that AMC-109 is also readily soluble in chloroform (data not shown).

### Paintings

TABLE F
The appearance of the painted surfaces.

| Sample | Appearance |
|---|---|
| Estane 58300 | Medium thick, flexible (stretchy), lightly opaque, |
| | no visual particles |
| Pearlcoat | Thin, stretchy, lightly opaque, particles inside (air |
| Dipp 119 | bubbles) |
| Tecoflex | Thin, flexible, lightly opaque, no visual particles |

### Extractions and Leakage Kinetics

The amount of AMC-109 in each of the extracts were calculated using the UV-spectrophotometry standard curve. The data were normalized to the total amount AMC-109 present in the original sample. The normalized data are compiled in Table G.

TABLE G
AMC-109 leakage at various timepoints (minutes)
expressed as % of the total amount in the sample.

| Time (min) | Pearlcoat | Tecoflex | Estane |
|---|---|---|---|
| 0.16 | 6.6 | 6.9 | 5.9 |
| 5 | 10.1 | 0.8 | 1.0 |
| 30 | 3.6 | 0.3 | 0.65 |
| 180 | 1.9 | 0.24 | 0.65 |
| 1320 | 0.9 | 0.4 | 0.81 |
| 2880 | 0.5 | 0.42 | 0.42 |

### Microbiological Efficacy

### Colony Forming Units

The material (i.e. the paint film containing AMC-109) was washed thoroughly for 2 minutes to remove AMC-109 that is readily extractable or residing directly on the surface. The number of CFU was below the detection limit for the AMC-109 containing material. Compared to the control material (i.e. relevant TPU paint without AMC-109) there was a 7 log reduction in CFU numbers (Table H).

TABLE H
CFU values for selected samples painted with AMC-109.

| TPU/polymer | AMC-109 | Control |
|---|---|---|
| Estane 58300 | 0 | 5.7 × 10⁷ |
| Tecoflex | 0 | 8.8 × 10⁷ |
| Pearlcoat Dipp | 0 | 3.8 × 10⁷ |

### Conclusions

TPUs can be dissolved by several solvents. Of the tested solvents, THF and dichloromethane have the most general applicability.
AMC-109 dissolved in solvent (e.g. THF) can be mixed into solvent (e.g. THF) dissolved polymers (TPUs).
The resulting painting solution can be applied on several surfaces.
The properties of the painted surfaces vary according to the polymer (TPU), different polymers may be suitable for different applications.
The painted surfaces (coatings) leak AMC-109 (i.e. AMC-109 can leach out), typically rapidly at first, but sustained at a low concentration over at least two days.
The painted surfaces are strongly anti-colonizing (even after being washed with water for 2 min to remove AMC-109 that is readily extractable or residing directly on the surface).

This study demonstrates that compositions comprising AMC-109, a TPU and an appropriate solvent(s) (e.g. THF), which may also be referred to as AMC-109 containing paints (or AMC-109/TPU containing paints), are useful as paints (or to provide coatings) for surfaces that may be susceptible to microbial contamination, as such paints (coatings) resist microbial growth thereon.

### Extension of the Study

In an extension to the study described above, experiments were also performed to assess the ability of samples of Tecoflex-AMC-109 and Pearlcoat Dipp 119-AMC-109 coatings/films (both of which were made using a painting solution made by mixing the TPU dissolved in THF with AMC-109 dissolved in THF) to resist colonisation by bacteria (S. aureus) even after said samples have been subjected to extractions of the AMC-109 (by shaking in water for different time periods). It was observed that such post-extraction samples of Tecoflex-AMC-109 paints were non-colonizable by S. aureus for a period of at least 20 hours and it was observed that such post-extraction samples of Pearlcoat Dipp 119—AMC-109 paints were non-colonizable by S. aureus for a period of at least 3 hours (data not shown).

### Example 11

The purpose of this study is to investigate whether an AMC-109 containing TPU (thermoplastic polyurethane) paint can be applied to a urinary catheter (in this example the urinary catheter is made of silicone) and whether this AMC 109/TPU paint confers antimicrobial properties.

### Materials and Methods (Preparation of Painting Solution and Painting Procedure)

### Sample Materials

Tecoflex TPU is a commercially available medical grade TPU (Lubrizol). Tecoflex is an aliphatic polyether-based thermoplastic polyurethane (TPU). The specific type of Tecoflex used in this study was Tecoflex EG-80A.

Pearlcoat DIPP 119 is a commercially available TPU (Lubrizol). Pearlcoat DIPP 119 is an aromatic polycaprolactone copolyester-based thermoplastic polyurethane (TPU).

### Preparation of Painting Solutions

Tecoflex (1.0 g) was dissolved in THF (tetrahydrofuran) (40 ml) or Pearlcoat DIPP 119 (1.0 g) was dissolved in THF (25 ml). AMC-109 (53 mg) was also dissolved in THF (4 ml). The AMC-109 dissolved in THF was then mixed with the TPU (i.e. Tecoflex or Pearlcoat DIPP 199) dissolved in THF. This mixture is referred to as a “final painting solution”. The concentration of AMC-109 in the final painting solution was 5% relative to the TPU. The dissolution of AMC-109 in THF takes several hours.

### Painting Procedure

A piece of a Covidien Foley catheter (a urinary catheter made of silicone) was divided lengthwise and dipped in either a 44 ml final painting solution of Tecoflex and AMC-109 in THF as described above, or in 29 ml final painting solution of Pearlcoat DIPP 119 and AMC-109 in THE as described above. The samples (i.e. the dipped pieces of catheter) were then dried. An equivalent coating made with a solution without AMC-109 was made as a control.

### Materials and Methods (Microbiology)

### Bacterial Strain:

Staphylococcus aureus 8325

### Modified AATCC-100 Method

Overnight colonies of S. aureus were diluted to 0.5 McFarland in 0.9% NaCl resulting in a bacterial concentration of 1.5×10⁸ CFU/ml. This suspension was further diluted in TSB (tryptic soy broth) to 1×10⁵ CFU/ml.

The test material (i.e. the dried catheter after painting) was cut in pieces of approximately 1 cm. The different samples were inoculated with 50 μl of the bacterial solution (i.e. 50 μl of the 1×10⁵ CFU/ml suspension) on the hollow side of the tube (i.e. the lumen side). The samples were placed on a glass slide, and incubated in a moisture chamber at 37° C. for 24 hours. Three biological replicates of each test material were made.

After incubation, the test material was placed in 900 μl NaCl (0.9%) and vortexed for 45 seconds, before making serial dilutions (0-10⁻⁶) and plating of 100 μl for CFU counting.

### Results

### Microbiological Efficacy

### Colony Forming Units

The number of CFU was below the detection limit for the AMC-109 containing material. Compared to the control material there was a 7 log reduction in CFU numbers, Table B.

TABLE B
CFU values for samples painted with TPU/AMC-109 paint.

| | AMC-109 (i.e. with | |
|---|---|---|
| | AMC-109 in the final | Control (i.e. |
| TPU/polymer | painting solution) | without AMC-109) |
| Tecoflex | 0 | 8.0 × 10⁷ |
| Pearlcoat Dipp 119 | 0 | 4.1 × 10⁷ |

### Conclusion

A paint composed of AMC-109 and a TPU dissolved in THF can successfully be applied as a paint to a silicone urinary catheter. This has been demonstrated with two different TPUs (Tecoflex and Pearlcoat Dipp 119).

The painted (i.e. coated) urinary catheters show excellent antimicrobial and anticolonizing behaviour.

This study demonstrates that compositions comprising AMC-109, a TPU and an appropriate solvent (e.g. THF), which may also be referred to as AMC-109 containing paints (or AMC-109/TPU containing paints), are useful for painting (or coating) articles (e.g. medical devices) that may be susceptible to microbial contamination, as such painted (coated) articles resist microbial growth thereon.

### Example 12

The purpose of this study is to investigate the release characteristics of AMC-109 from an AMC-109 containing thermoplastic polyurethane (Tecoflex) paint (i.e. from a surface painted with such a paint). Antimicrobial properties of the AMC-109 containing thermoplastic polyurethane (TPU) paint are also investigated.

### Materials and Methods

### Sample Materials

A stock painting solution (paint) of Tecoflex EG 80A (Lubrizol Advanced Materials, Inc.) and AMC-109 was prepared by mixing 920 mg of Tecoflex in 30 ml of THF (tetrahydrofuran) with 50 mg of AMC-109 in 1 ml of THF. The stock painting solution was divided in three:
10 ml stock Tecoflex and AMC-109 paint (approx. 5% AMC-109 relative to Tecoflex)
10 ml stock Tecoflex and AMC-109 paint+PEG400 (112 mg)
10 ml stock Tecoflex and AMC-109 paint+PEG1000 (108 mg)

The PEG, added to the paints in two of the three cases as set out above as it could be a release enhancer, had either an average molecular mass of 400 (PEG400) or 1000 (PEG1000). Thin films of the AMC-109 containing Tecoflex paint were made by dipping Tecoflex samples (square sheets cut with edges approx. 1.5 cm) into the paint solutions and leaving to dry hanging from clips. For the avoidance of doubt, in these experiments, Tecoflex was the TPU used in the paints, and Tecoflex was also the sample material to which the paint was the applied (i.e. Tecoflex was also the material that was painted with the AMC-109 containing Tecoflex paint).

### AMC-109 Release Experiments (Extraction Experiments)

Each painted sample was cut into three equally sized parts and placed in a vial containing 1.5 ml of water. The vials were placed on an orbital shaker. At a series of time intervals (10 min, 3 h, 20 h, 2 d, 5 d, 8 d, 14 d, and 18 d), the aqueous extract was sucked off and replaced with 1.5 ml fresh water and the vials were returned to the shaker. The aqueous extracts taken at each time interval were analysed for AMC-109 content by a UV-Vis method (i.e. the amount of AMC-109 in each extract was determined by UV-spectrophotometry at 280 nm using a pre-made standard curve).

### Microbiology

### Bacterial Strains:

Staphylococcus aureus 8325
E. coli

### Modified AATCC-100

Overnight colonies of S. aureus were diluted to 0.5 McFarland in 0.9% NaCl resulting in a bacterial concentration of 1.5×10⁸ CFU/ml. This solution was further diluted in TSB (tryptic soy broth) to 1×10⁵ CFU/ml. Analogous was done for E. coli.

The test materials (i.e. painted samples) were cut in pieces of approximately 1×1 cm. The different samples (test materials) were inoculated with 50 μl of the bacterial suspension (1×10⁵ CFU/ml). The samples were incubated in a moisture chamber at 37° C. for 24 hours. Three biological replicates of each test material were made.

After incubation, the test materials were placed in 900 μl NaCl and vortexed for 45 seconds, before making serial dilutions (0-10⁻⁶) and plating of 100 μl for CFU counting.

### Results

### Production of Painted Coupons

For each type of paint (i.e. for each of the three types of painting solution set out in the bullet points above), a set of triplicate painted samples was prepared. Thus, there were three sets of painted samples (with three samples per set) that were painted with an AMC-109 containing paint (active paints). There was also one set of painted samples (with three samples in the set) prepared in which the paint was a Tecoflex-only paint (i.e. no AMC-109 was included); this was for a control in the microbiology experiments. The mass of the painted samples varied between 682 mg and 772 mg, and the paint layer between 6.09 mg and 8.87 mg. The paint layer mass for each active painted sample is compiled in Table I below. The paint layer thickness is calculated to be between 10-20 μm, however there is very likely a great inhomogeneity in the layer thickness.

Aqueous Extraction (Release) The painted samples were extracted in vials containing 1.5 ml of water (extraction in water here means that the samples were exposed to water to allow the AMC-109 to be released from, or extracted from, the paint layer). The amount of water (1.5 ml) was chosen to represent “sink”-conditions while simultaneously being a volume of water that allows the amount of AMC-109 released (or extracted) into the water to be measured by the UV-method (if too large a volume of water is used it could be difficult to measure the released/extracted AMC-109). Each extract (except the first) contained less than 10 μg AMC-109 in 1.5 ml of water, indicating that any back-flow of AMC-109 into the paint would at most be marginal. As described above, at each time-point the aqueous extract was exchanged with fresh water. Extraction data is shown below in Table I.

TABLE I
Amount AMC-109 extractable (mg/h) from the AMC-109 containing Tecoflex EG 80A paint at various time points
(rows 3-10), total amount AMC-109 extracted in mg (row 11, “Total”), mass of the paint of each sample
in mg (row 12), calculated mass (in mg) of AMC-109 in the paint for each sample (row 13), and percent AMC-
109 recovery in each sample (row 14). Percent AMC-109 recovery (Recovery %) is the percentage of the AMC-109
in the paint that was extracted from (released from) the paint by day 18. The columns headed 1, 2 and 3 are
experimental replicates with samples painted with the AMC-109 containing paint described in the first bullet
point in the “Sample materials” section above. The columns headed 4, 5 and 6 are experimental replicates
with samples painted with the AMC-109 containing paint described in the second bullet point in the “Sample
materials” section above. The columns headed 7, 8 and 9 are experimental replicates with samples painted
with the AMC-109 containing paint described in the third bullet point in the “Sample materials” section above.

| | AMC-109 | | | AMC-109 + PEG400 | | | AMC-109 + PEG1000 | | |
|---|---|---|---|---|---|---|---|---|---|
| Time (d) | 1 | 2 | 3 | 4 | 5 | 6 | 7 | 8 | 9 |
| 0.0067 | 0.13125 | 0.12188 | 0.10313 | 0.10313 | 0.11250 | 0.12188 | 0.12188 | 0.12188 | 0.12188 |
| 0.1316 | 0.00150 | 0.00150 | 0.00150 | 0.00200 | 0.00250 | 0.00250 | 0.00250 | 0.00250 | 0.00250 |
| 0.965 | 0.00053 | 0.00045 | 0.00045 | 0.00045 | 0.00045 | 0.00045 | 0.00053 | 0.00053 | 0.00053 |
| 2 | 0.00038 | 0.00038 | 0.00038 | 0.00031 | 0.00038 | 0.00038 | 0.00038 | 0.00031 | 0.00031 |
| 5 | 0.00021 | 0.00021 | 0.00023 | 0.00019 | 0.00019 | 0.00019 | 0.00019 | 0.00017 | 0.00019 |
| 8 | 0.00019 | 0.00023 | 0.00019 | 0.00017 | 0.00017 | 0.00017 | 0.00017 | 0.00015 | 0.00015 |
| 14 | 0.00013 | 0.00014 | 0.00011 | 0.00011 | 0.00014 | 0.00013 | 0.00011 | 0.00010 | 0.00011 |
| 18 | 0.00013 | 0.00013 | 0.00014 | 0.00011 | 0.00013 | 0.00011 | 0.00009 | 0.00009 | 0.00011 |
| Total | 0.1035 | 0.105 | 0.099 | 0.0915 | 0.1005 | 0.099 | 0.0975 | 0.0915 | 0.096 |
| Paint | 7.38 | 7.29 | 6.09 | 6.53 | 7.46 | 7.94 | 7.26 | 7.50 | 8.87 |
| mass | | | | | | | | | |
| AMC-109 | 0.3801 | 0.3754 | 0.3136 | 0.3363 | 0.3842 | 0.4089 | 0.3739 | 0.3863 | 0.4568 |
| mass | | | | | | | | | |
| Recovery | 27.2 | 28.0 | 31.6 | 27.2 | 26.2 | 24.2 | 26.1 | 23.7 | 21.0 |
| (%) | | | | | | | | | |

The amount extracted AMC-109 was converted into the amount extractable/h (mg/h) to compensate for the uneven sampling rate.

Raw data are shown In Table J below.

TABLE J
Raw data: AMC-109 content (mg/ml) in extracts at various time points (rows 2-9); Total recovery in mg (i.e. total amount (in mg) AMC-109
extracted; row 10); % Recovery (percentage of the AMC-109 in the paint that was extracted from (released from) the paint by day 18; row
11); Extractable amount (mg/h) (rows 13-20), Mass of paint (mg) (row 23); and calculated mass of AMC-109 in the paint (mg) (row 24).

| | Total | Delta | | | | Tecoflex + | Tecoflex + | Tecoflex | Tecoflex + | Tecoflex + | Tecoflex + |
|---|---|---|---|---|---|---|---|---|---|---|---|
| | Time | time | Tecoflex + | Tecoflex + | Tecoflex + | AMC + | AMC + | AMC + | AMC + | AMC + | AMC + |
| | (d) | (h) | AMC | AMC | AMC | PEG400 | PEG400 | PEG400 | PEG1000 | PEG1000 | PEG1000 |
| Raw data | 0.0067 | 0.16 | 0.014 | 0.013 | 0.011 | 0.011 | 0.012 | 0.013 | 0.013 | 0.013 | 0.013 |
| (mg/ml) | 0.1316 | 3 | 0.003 | 0.003 | 0.003 | 0.004 | 0.005 | 0.005 | 0.005 | 0.005 | 0.005 |
| | 0.965 | 20 | 0.007 | 0.006 | 0.006 | 0.006 | 0.006 | 0.006 | 0.007 | 0.007 | 0.007 |
| | 2 | 24 | 0.006 | 0.006 | 0.006 | 0.005 | 0.006 | 0.006 | 0.006 | 0.005 | 0.005 |
| | 5 | 72 | 0.010 | 0.010 | 0.011 | 0.009 | 0.009 | 0.009 | 0.009 | 0.008 | 0.009 |
| | 8 | 72 | 0.009 | 0.011 | 0.009 | 0.008 | 0.008 | 0.008 | 0.008 | 0.007 | 0.007 |
| | 14 | 144 | 0.012 | 0.013 | 0.011 | 0.011 | 0.013 | 0.012 | 0.011 | 0.010 | 0.011 |
| | 18 | 96 | 0.008 | 0.008 | 0.009 | 0.007 | 0.008 | 0.007 | 0.006 | 0.006 | 0.007 |
| Total | | | 0.1035 | 0.105 | 0.099 | 0.0915 | 0.1005 | 0.099 | 0.0975 | 0.0915 | 0.096 |
| Recovery | | | | | | | | | | | |
| (mg) | | | | | | | | | | | |
| Recovery | | | 27.2 | 28.0 | 31.6 | 27.2 | 26.2 | 24.2 | 26.1 | 23.7 | 21.0 |
| (%) | | | | | | | | | | | |
| Extractable | 0.0067 | | 0.13125 | 0.12188 | 0.10313 | 0.10313 | 0.11250 | 0.12188 | 0.12188 | 0.12188 | 0.12188 |
| amount | 0.1316 | | 0.00150 | 0.00150 | 0.00150 | 0.00200 | 0.00250 | 0.00250 | 0.00250 | 0.00250 | 0.00250 |
| (mg/h) | 0.965 | | 0.00053 | 0.00045 | 0.00045 | 0.00045 | 0.00045 | 0.00045 | 0.00053 | 0.00053 | 0.00053 |
| | 2 | | 0.00038 | 0.00038 | 0.00038 | 0.00031 | 0.00038 | 0.00038 | 0.00038 | 0.00031 | 0.00031 |
| | 5 | | 0.00021 | 0.00021 | 0.00023 | 0.00019 | 0.00019 | 0.00019 | 0.00019 | 0.00017 | 0.00019 |
| | 8 | | 0.00019 | 0.00023 | 0.00019 | 0.00017 | 0.00017 | 0.00017 | 0.00017 | 0.00015 | 0.00015 |
| | 14 | | 0.00013 | 0.00014 | 0.00011 | 0.00011 | 0.00014 | 0.00013 | 0.00011 | 0.00010 | 0.00011 |
| | 18 | | 0.00013 | 0.00013 | 0.00014 | 0.00011 | 0.00013 | 0.00011 | 0.00009 | 0.00009 | 0.00011 |
| Extraction | 1.5 | | | | | | | | | | |
| volume | | | | | | | | | | | |
| (ml) | | | | | | | | | | | |
| Sample | | | 1 | 2 | 3 | 4 | 5 | 6 | 7 | 8 | 9 |
| No. | | | | | | | | | | | |
| Mass of | | | 7.38 | 7.29 | 6.09 | 6.53 | 7.46 | 7.94 | 7.26 | 7.50 | 8.87 |
| paint (mg) | | | | | | | | | | | |
| Mass AMC- | | | 0.3801 | 0.3754 | 0.3136 | 0.3363 | 0.3842 | 0.4089 | 0.3739 | 0.3863 | 0.4568 |
| 109 in the | | | | | | | | | | | |
| paint (mg) | | | | | | | | | | | |

After 18 d of aqueous extraction, the experiment was stopped, and the painted samples were tested microbiologically using a modified AATCC-100 method. The painted samples had at this time lost ˜30% of the AMC-109 in the paint layer.

### Anti-Colonizing Efficacy

The anti-colonizing efficacy after 18 d continuous aqueous extraction was assessed against S. aureus and E. coli using a modified AATCC-100 method. In this method a bacterial inoculum is placed directly on top on the surface of the painted sample and incubated for 24 h at 37° C. The antimicrobial effect on the inoculum was measured by determining the number of colony forming units (CFU) at the end of the incubation.

The results for anti-colonizing efficacy against S. aureus are shown below in Table K.

TABLE K
Anti-colonizing efficacy against S. aureus
as determined by the modified AATCC-100 method.

| Sample type | Colony forming units |
|---|---|
| AMC-109 | 0 |
| AMC-109 + PEG400 | 0 |
| AMC-109 + PEG1000 | 0 |
| Tecoflex | 7.0 × 10⁷ |
| Material only | 7.1 × 10⁷ |

The results in Table K show that after exposure to water for at least 18 days, surfaces painted with AMC-109 containing TPU paints remain non-colonizable by S. aureus.

The results for anti-colonizing efficacy against E. coli are shown below in Table L.

TABLE L
Anti-colonizing efficacy against E. coli as
determined by the modified AATCC-100 method.

| Sample type | Colony forming units |
|---|---|
| AMC-109 | 0 |
| AMC-109 + PEG400 | 0, 0, 8.2 × 10⁷* |
| AMC-109 + PEG1000 | 0 |
| Control | 8.4 × 10⁷ |

*Outlier probably caused by the incomplete painting (incomplete coating) of the surface of one of the samples tested (one of the three replicates).

The results in Table L show that after exposure to water for at least 18 days, surfaces painted with AMC-109 containing TPU paints remain non-colonizable by E. coli.

For the avoidance of doubt, the “AMC-109” sample type in Tables K and L is sample painted with the AMC-109 containing TPU paint described in the first bullet point in the “Sample materials” section above. The “AMC-109+PEG400” sample type in Tables K and L is sample painted with the AMC-109 containing TPU paint described in the second bullet point in the “Sample materials” section above. The “AMC-109+PEG1000” sample type in Tables K and L is sample painted with the AMC-109 containing TPU paint described in the third bullet point in the “Sample materials” section above. The “Tecoflex” sample type in Table K is sample painted with a Tecoflex-only paint (i.e. no AMC-109 was included in the paint). The “Material only” sample type in Table K is unpainted sample (i.e. unpainted Tecoflex material). The “Control” sample type in Table L is sample painted with a Tecoflex-only paint (i.e. no AMC-109 was included in the paint).

### Conclusion

The aqueous extraction experiment was set up to mimic a situation where the device is subjected to a continuous flow of water for 18 days. The results shows that, on average, slightly less than 30% of the AMC-109 content is extracted during this period. Of the total extracted amount, 12-15% is liberated after 10 min, whereas the rest (85-88%) is slowly liberated at an almost constant rate for the rest of the extraction period. The data demonstrates that painting with AMC-109 containing TPU paints is fully capable of hindering bacterial colonization of painted surfaces, even after almost 3 weeks of continuous flushing.

The main results of the present study are:
Thin films of Tecoflex containing AMC-109 can be applied on surfaces using a dipping technique. Accordingly, surfaces of materials can be painted with TPU paints that contain AMC-109 to provide materials with a painted surface.
The release (or extraction) of AMC-109 is by far the largest within the first 10 min, and levels out on a low, but steady level lasting at least 18 d.
The surface of the thin TPU film (or paint layer) containing AMC-109 is non-colonizable by S. aureus or E. coli for a period of at least 18 d.
The mass balance suggests that, on average, slightly less than 30% of the AMC-109 present in Tecoflex paint layer leaks out of the material (i.e. is released or extracted from the material) within 18 d, and that 12-15% of this amount is liberated within 10 min, with the rest leaking out slowly at an almost constant rate. The data thus shows that there is a sustained release of AMC-109 over time.

1. An antimicrobial article comprising polyurethane, wherein said polyurethane
is impregnated with a compound of Formula (I)
AA-AA-AA-X—Y (I)
wherein, in any order, 2 of said AA (amino acid) moieties are cationic amino
acids and 1 of said AA is an amino acid with a lipophilic R group, the R group having 14-27 non-hydrogen atoms;
X is a N atom, which may be substituted by a branched or unbranched C1-C10 alkyl or aryl group, which group may incorporate up to 2 heteroatoms selected from N, O and S; and
Y is selected from the group consisting of R1-R2-R3,
R1-R2-R2-R3, R2-R2-R1-R3, R1-R3, and R4
wherein:
R1 is C, O, S or N, R2 is C;
each of R1 and R2 may be substituted by C1-C4 alkyl groups or unsubstituted;
R3 is a group comprising 1 to 3 cyclic groups each of 5 or 6 non-hydrogen atoms, 2 or more of the cyclic groups may be fused and one or more of the cyclic groups may be substituted; R3 incorporates a maximum of 15 non-hydrogen atoms; and
R4 is an aliphatic moiety having 2-20 non-hydrogen atoms, said moiety being linear branched or cyclic.

2. The antimicrobial article of claim 1, wherein said compound is a peptide.

3. The antimicrobial article of claim 1, wherein said cationic amino acids are arginine and/or lysine.

4. The antimicrobial article of claim 1, wherein said amino acid with a lipophilic R group is selected from tributyl tryptophan (Tbt) or a biphenylalanine derivative selected from Phe (4-(2-Naphthyl)), Phe (4-(1-Naphthyl)), Bip (4-n-Bu), Bip (4-Ph) or Bip (4-T-Bu); Bip (4-(2-Naphthyl)).

5. The antimicrobial article of claim 1, wherein said compound is a compound of formula (II)
AA1-AA2-AA1-X—Y (II)
wherein:
AA1 is a cationic amino acid;
AA2 is an amino acid with a lipophilic R group, the R group having 14-27 non-hydrogen atoms; and
X and Y are as defined in claim 1.

6. The antimicrobial article of claim 1, wherein said compound has the structural formula

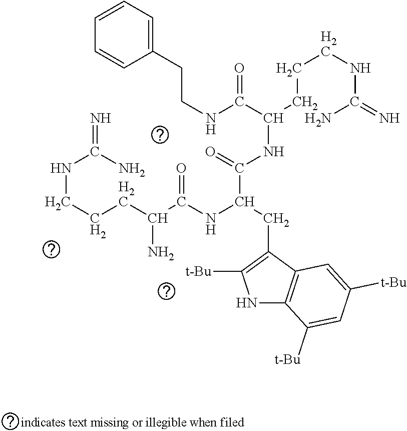 [SMILES: CC(C)(C)c1cc(C(C)(C)C)c2[nH]c(C(C)(C)C)c(CC(NC(=O)C(N)CCCNC(=N)N)C(=O)NC(CCCNC(=N)N)C(=O)NCCc3ccccc3)c2c1]

7. The antimicrobial article of claim 1, wherein said polyurethane is a thermoplastic polyurethane (TPU).

8. The antimicrobial article of claim 1, wherein said polyurethane is not in the form of a foam.

8. The antimicrobial article of claim 1, wherein said polyurethane impregnated with said compound is in the form of a coating on said article.

10. The antimicrobial article of claim 1, wherein said antimicrobial article is a medical device.

11. The antimicrobial article of claim 1, wherein said antimicrobial article is a wound dressing.

12. The antimicrobial article of claim 1, wherein said antimicrobial article is an in-dwelling medical device.

13. The antimicrobial article of claim 1, wherein said antimicrobial article is a catheter.

14. Polyurethane impregnated with a compound as defined in claim 1.

15. (canceled)

16. A method of producing polyurethane, that is not in the form of foam,
impregnated with a compound as defined in claim 1, said method comprising (i) applying to said polyurethane a solution of the compound in which solution the solvent is an organic solvent and (ii) drying the polyurethane to which said solution has been applied, thereby producing said polyurethane impregnated with the compound.

17. Polyurethane impregnated with a compound, wherein said polyurethane is produced by the method of claim 16.

18. Polyurethane impregnated with a compound as defined in claim 1, wherein said polyurethane is not in the form of a foam and said compound is impregnated into said polyurethane over only a portion of the total depth from the surface of the polyurethane.

19. A composition comprising at least one organic solvent, a thermoplastic polyurethane (TPU) and a compound as defined in claim 1, wherein said TPU and said compound are each dissolved in said at least one organic solvent.

20. The composition of claim 19, wherein said organic solvent is a polar aprotic solvent.

21. The composition of claim 19, wherein the organic solvent is tetrahydrofuran (THF), dichloromethane (DCM), acetone or ethyl acetate.

22. The composition of claim 19, wherein the organic solvent is THF.

23. A method of producing a composition of claim 19, said method comprising:
(i) dissolving a thermoplastic polyurethane (TPU) in an organic solvent;
(ii) dissolving a compound as defined in any preceding claim in a solvent;
(iii) mixing the TPU dissolved in the organic solvent obtained in (i) with the compound dissolved in the solvent obtained in (ii), thereby producing said composition.

24. The method of claim 23, wherein the solvent of (ii) is an organic solvent.

25. The method of claim 23, wherein the solvent of (i) and/or (ii) is tetrahydrofuran (THF), dichloromethane (DCM), acetone, or ethyl acetate.

26. The method of claim 23, wherein the solvent of (i) is the same as the solvent of (ii).

27. The method of claim 23, wherein the solvent of (i) is different from the solvent of step (ii).

28. A method of producing a composition of claim 19, said method comprising (i) providing a first solution comprising a thermoplastic polyurethane (TPU) in an organic solvent; (ii) providing a second solution comprising a compound of Formula (I), wherein said second solution is miscible with the first solution; and (iii) mixing said first and second solutions.

29. The method of claim 28, wherein said organic solvent is a polar aprotic solvent, tetrahydrofuran (THF), dichloromethane (DCM), acetone or ethyl acetate.

30. The method of claim 28, wherein said second solution Comprises a compound of Formula (I) and an organic solvent.

31. (canceled)

32. (canceled)

32. A method of producing a coating for an article or surface that may be susceptible to microbial contamination, said method comprising (i) applying a composition of claim 19 to said article or surface, and (ii) evaporating off the solvent from said applied composition.

33. A coating for an article or surface that may be susceptible to microbial contamination, wherein said coating comprises thermoplastic polyurethane impregnated with a compound of Formula (I).

34. The coating of claim 33, wherein said coating is produced by applying a composition comprising at least one organic solvent, a thermoplastic polyurethane (TPU) and the compound to said article or surface and evaporating off the solvent from said applied composition.

35. A method of producing an article, or a surface, having an antimicrobial coating comprising thermoplastic polyurethane impregnated with a compound as defined in claim 1, said method comprising (i) providing a composition of comprising at least one organic solvent, a thermoplastic polyurethane (TPU); and (ii) applying said composition to the article or surface, or to at least a part of the article or surface.

36. The method of claim 35, wherein said applying is by dipping the article or surface into said composition or by painting said composition onto said article or surface.

37. An article that may be susceptible to microbial contamination that is coated, or at least partially coated, with a coating composition of claim 19.

38. (canceled)

39. The coating of claim 33, wherein said article is a medical device.

40. (canceled)

41. The polyurethane of claim 14, wherein said polyurethane comprises polyol-derived parts that allow the polyurethane to absorb water.

42. The polyurethane of claim 41, wherein said polyol is a polyether polyol.

43. The article of claim 37, wherein said polyurethane is produced by the reaction of a polyether polyol with an aliphatic diisocyanate.

44. The article of claim 37, wherein, in use, there is sustained release of said compound of Formula (I) from the polyurethane.

45. (canceled)

46. (canceled)

47. (canceled)

48. A method of treating or preventing an infection which method comprises applying to subject in need thereof a therapeutically effective amount of a compound as defined in claim 1, wherein said compound is administered to said subject in the form of an antimicrobial article comprising polyurethane that is impregnated with said compound.

49. (canceled)

50. The the method of claim 48, wherein said infection is a bacterial infection.
